(19) United States
(12) Patent Application Publication
(10) Pub. No.: US 2008/0015146 A1
(43) Pub. Date: Jan. 17, 2008

(54) Dpp-IV Inhibitors

(75) Inventors: Paul John Edwards, Laval (CA); Silvia Cerezo-Galvez, Wuppertal (DE); Achim Feurer, Auggen (DE); Victor Giulio Matassa, Barcelona (ES); Sonja Nordhoff, Arlesheim (CH); Stephan Bulat, Heidelberg-Ziegelhausen (DE); Meritxell Lopez-Canet, Dusseldorf (DE); Christian Rummey, Basel (CH); Claudia Rosenbaum, Hurth (DE)

Correspondence Address: KILYK & BOWERSOX, P.L.L.C., 400 HOLIDAY COURT, SUITE 102, WARRENTON, VA, 20186, US

(73) Assignee: SANTHERA PHARMACEUTICALS (DEUTSCHLAND) AG, Heidelberg (DE)

(21) Appl. No.: 11/569,938

(22) PCT Filed: Jun. 8, 2005
(86) PCT No.: PCT/EP05/06162

§ 371 Date: May 10, 2007

(30) Foreign Application Priority Data
Jun. 8, 2004 (EP) 04013512.1

(51) Int. Cl. A61K 31/40; A61K 31/4245; A61K 31/4406; A61K 31/443; A61K 38/02; A61K 38/28; A61P 1/00; A61P 25/00; A61P 31/18; A61P 35/00; A61P 7/00; C07D 207/09; C07D 271/06; C07D 413/14

(52) U.S. Cl. 514/12; 514/2; 514/340; 514/356; 514/364; 514/422; 544/359; 546/268.1; 546/269.4; 548/131; 548/364.1; 548/518

(57) ABSTRACT

The invention relates to compounds of formula (I) 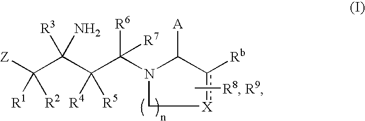
wherein Z, R¹⁻⁹, n, A, X and R^{b} have the meaning as cited in the description and the claims. Said compounds are useful as DPP-IV inhibitors. The invention also relates to the preparation of such compounds as well as the production and use thereof as medicament.

The present invention relates to a novel class of dipeptidyl peptidase inhibitors, including pharmaceutically acceptable salts and prodrugs thereof, which are useful as therapeutic compounds, particularly in the treatment of Type 2 diabetes mellitus, often referred to as non-insulin dependent diabetes mellitus (NIDDM), and of conditions that are often associated with this disease, such as obesity and lipid disorders.

Diabetes refers to a disease process derived from multiple causative factors and characterized by elevated levels of plasma glucose or hyperglycemia in the fasting state or after administration of glucose during an oral glucose tolerance test. Persistent or uncontrolled hyperglycemia is associated with increased and premature morbidity and mortality. Often abnormal glucose homeostasis is associated both directly and indirectly with alterations of the lipid, lipoprotein and apolipoprotein metabolism and other metabolic and hemodynamic disease. Therefore patients with Type 2 diabetes mellitus are at an increased risk of macrovascular and microvascular complications, including coronary heart disease, stroke, peripheral vascular disease, hypertension, nephropathy, neuropathy, and retinopathy. Therefore, therapeutic control of glucose homeostasis, lipid metabolism and hypertension are critically important in the clinical management and treatment of diabetes mellitus.

There are two generally recognized forms of diabetes. In Type 1, or insulin-dependent, diabetes mellitus (IDDM), patients produce little or no insulin, which is the hormone regulating glucose utilization. In Type 2, or noninsulin dependent, diabetes mellitus (NIDDM), patients often have plasma insulin levels that are the same or elevated compared to nondiabetic subjects. These patients develop a resistance to the insulin stimulating effect on glucose and lipid metabolism in the main insulin-sensitive tissues, namely the muscle, liver and adipose tissues. Further, the plasma insulin levels, while elevated, are insufficient to overcome the pronounced insulin resistance.

Insulin resistance is not primarily due to a diminished number of insulin receptors but to a post-insulin receptor binding defect that is not yet understood. This resistance to insulin responsiveness results in insufficient insulin activation of glucose uptake, oxidation and storage in muscle, and inadequate insulin repression of lipolysis in adipose tissue and of glucose production and secretion in the liver.

The available treatments for Type 2 diabetes, which have not changed substantially in many years, have recognized limitations. While physical exercise and reductions in dietary intake of calories will dramatically improve the diabetic condition, compliance with this treatment is very poor because of well-entrenched sedentary lifestyles and excess food consumption, especially of foods containing high amounts of saturated fat. Increasing the plasma level of insulin by administration of sulphonylureas (e.g., tolbutamide and glipizide) or meglitinide, which stimulate the pancreatic β-cells to secrete more insulin, and/or by injection of insulin when sulphonylureas or meglitinide become ineffective, can result in insulin concentrations high enough to stimulate the very insulin-resistant tissues. However, dangerously low levels of plasma glucose can result from administration of insulin or insulin secretagogues (sulphonylureas or meglitinide), and an increased level of insulin resistance, due to the even higher plasma insulin levels, can occur. The biguanides increase insulin sensitivity resulting in some correction of hyperglycemia. However, the two biguanides, phenformin and metformin, can induce lactic acidosis and nausea/diarrhea. Metformin has fewer side effects than phenformin and is often prescribed for the treatment of Type 2 diabetes.

The glitazones (i.e., 5-benzylthiazolidine-2,4-diones) are a recently described class of compounds with potential for ameliorating many symptoms of Type 2 diabetes. These agents substantially increase insulin sensitivity in muscle, liver and adipose tissue in several animal models of Type 2 diabetes, resulting in partial or complete correction of the elevated plasma levels of glucose without occurrence of hypoglycemia. The glitazones that are currently marketed are agonists of the peroxisome proliferator activated receptor (PPAR), primarily the PPAR-gamma subtype. PPAR-gamma agonism is generally believed to be responsible for the improved insulin sensitization that is observed with the glitazones. Newer PPAR agonists that are being tested for treatment of Type 2 diabetes are agonists of the alpha, gamma or delta subtype, or a combination of these, and in many cases are chemically different from the glitazones (i.e., they are not thiazolidinediones). Serious side effects (e.g., liver toxicity) have occurred with some of the glitazones, such as troglitazone.

Additional methods of treating the disease are still under investigation. New biochemical approaches that have been recently introduced or are still under development include treatment with alpha-glucosidase inhibitors (e.g., acarbose) and protein tyrosine phosphatase-IB (PTP-1B) inhibitors.

Compounds that are inhibitors of the dipeptidyl peptidase-IV (DPP-IV) enzyme are also under investigation as drugs that may be useful in the treatment of diabetes, and particularly Type 2 diabetes. See for example WO-A-97/40832, WO-A-98/19998, WO-A-03/180, WO-A-03/181 and WO-A-2004/007468. The usefulness of DPP-IV inhibitors in the treatment of Type 2 diabetes is based on the fact that DPP-IV in vivo readily inactivates glucagon like peptide-1 (GLP-1) and gastric inhibitory peptide (GIP). GLP-1 and GIP are incretins and are produced when food is consumed. The incretins stimulate production of insulin. Inhibition of DPP-IV leads to decreased inactivation of the incretins, and this in turn results in increased effectiveness of the incretins in stimulating production of insulin by the pancreas. DPP-IV inhibition therefore results in an increased level of serum insulin. Advantageously, since the incretins are produced by the body only when food is consumed, DPP-IV inhibition is not expected to increase the level of insulin at inappropriate times, such as between meals, which can lead to excessively low blood sugar (hypoglycemia). Inhibition of DPP-IV is therefore expected to increase insulin without increasing the risk of hypoglycemia, which is a dangerous side effect associated with the use of insulin secretagogues.

DPP-IV inhibitors may also have other therapeutic utilities, as discussed elsewhere in this application. DPP-IV inhibitors have not been studied extensively to date, especially for utilities other than diabetes. New compounds are needed so that improved DPP-IV inhibitors can be found for the treatment of diabetes and potentially other diseases and conditions.

Thus, the object of the present invention is to provide a new class of DPP-IV inhibitors which may be effective in the treatment of Type 2 diabetes and other DPP-IV modulated diseases.

Accordingly, the present invention provides novel compounds of formula (I): 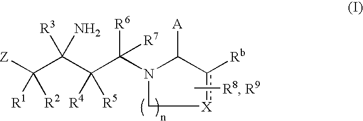
or a pharmaceutically acceptable salt thereof, wherein a dotted line indicates an optionally present double bond and wherein

Z is selected from the group consisting of phenyl; naphthyl; indenyl; C₃₋₇ cycloalkyl; indanyl; tetralinyl; decalinyl; heterocycle; and heterobicycle, wherein Z is optionally substituted with one or more R¹⁰, wherein R¹⁰ is independently selected from the group consisting of halogen; CN; OH; NH₂; oxo (═O), where the ring is at least partially saturated; R¹¹; and R¹²;

R¹¹ is selected from the group consisting of C₁₋₆ alkyl; O—C₁₋₆ alkyl; and S—C₁₋₆ alkyl, wherein R¹¹ is optionally interrupted by oxygen and wherein R¹¹ is optionally substituted with one or more halogen independently selected from the group consisting of F; and Cl;

R¹² is selected from the group consisting of phenyl; heterocycle; and C₃₋₇ cycloalkyl,

wherein R¹² is optionally substituted with one or more R¹³, wherein R¹³ is independently selected from the group consisting of halogen; CN; OH; NH₂; oxo (═O), where the ring is at least partially saturated; C₁₋₆ alkyl; O—C₁₋₆ alkyl; and S—C₁₋₆ alkyl;

R¹, R⁴ are independently selected from the group consisting of H; F; OH; and R¹⁴;

R², R⁵, R⁶, R⁷ are independently selected from the group consisting of H; F; and R¹″;

R¹⁴ is independently selected from the group consisting of C₁₋₆ alkyl; O—C₁₋₆ alkyl; N(R^{14a})—C₁₋₆ alkyl; S—C₁₋₆ alkyl; C₃₋₇ cycloalkyl; O—C₃₋₇ cycloalkyl; N(R^{14a})—C₃₋₇ cycloalkyl; S—C₃₋₇ cycloalkyl; —C₁₋₆ alkyl-C₃₋₇ cycloalkyl; O—C₁₋₆ alkyl-C₃₋₇ cycloalkyl; N(R^{14a})—C₁₋₆ alkyl-C₃₋₇ cycloalkyl; S—C₁₋₆ alkyl-C₃₋₇ cycloalkyl; heterocycle; O-heterocycle; N(R^{14a})-heterocycle; S-heterocycle; C₁₋₆ alkyl-heterocycle; O—C₁₋₆ alkyl-heterocycle; N(R^{14a})—C₁₋₆ alkyl-heterocycle; S—C₁₋₆ alkyl-heterocycle; wherein R¹⁴ is optionally substituted with one or more halogen independently selected from the group consisting of F; and Cl;

R^{14a} is selected from the group consisting of H; and C₁₋₆ alkyl;

Optionally R⁶ is selected from the group consisting of —C(R^{6a}R^{6b})—O—C₁₋₆ alkyl; —C(R^{6a}R^{6b})—O—C₃₋₇ cycloalkyl; —C(R^{6a}R^{6b})—S—C₁₋₆ alkyl; —C(R^{6a}R^{6b})—S—C₃₋₇ cycloalkyl; —C(R^{6a}R^{6b})—N(R^{6c})—C₁₋₆ alkyl; and —C(R^{6a}R^{6b})—N(R^{6c})—C₃₋₇ cycloalkyl, wherein each C₁₋₆ alkyl and C₃₋₇ cycloalkyl is optionally substituted with one or more R^{6d}, wherein R^{6d} is independently selected from the group consisting of halogen; C₁₋₆ alkyl; and C₃₋₇ cycloalkyl;

R^{6a}, R^{6b}, R^{6c} are independently selected from the group consisting of H; and C₁₋₆ alkyl;

R¹⁵ is independently selected from the group consisting of C₁₋₆ alkyl; C₃₋₇ cycloalkyl; and —C₁₋₆ alkyl-C₃₋₇ cycloalkyl, wherein R¹⁵ is optionally substituted with one or more R¹⁵, wherein R^{15a} is independently selected from the group consisting of F; Cl; and OH;

R³ is selected from the group consisting of H; and C₁₋₆ alkyl;

optionally one or more pairs of R¹, R², R³, R⁴, R⁵, R⁶, R^{6a}, R^{6b}, R⁷ independently selected from the group consisting of R¹/R²; R²/R³; R³/R⁴; R⁴/R⁵; R⁵/R⁶; R^{6a}/R^{6b} and R⁶/R⁷ form a C₃₋₇ cycloalkyl ring, which is optionally substituted with one or more of R^{15b}, wherein R^{15b} is independently selected from the group consisting of F; Cl; and OH;

optionally R⁶, R⁷ jointly form an oxo (═O);

n is 0, 1, 2 or 3;

X is selected from the group consisting of —C(R¹⁶R^{c})—; —C(R^{a})═CR^{c}—; —C(R¹⁶R^{a})—CR^{c}═, —C(R¹⁶R^{a})—O—; C(R¹⁶R^{a})—S—; —C(R¹⁶R^{a})—S(O)—; —C(R¹⁶R^{a})—S(O)₂—; —C(R¹⁶R^{a})—NR^{c}—; and —C(R¹⁶R^{a})—CR¹⁷R^{c}—;

R⁸ is selected from the group consisting of H; F; OH; and C₁₋₆ alkyl, optionally substituted with one or more halogen selected from the group consisting of F; and Cl;

R⁹, R¹⁶, R¹⁷ are independently selected from the group consisting of H; F; and C₁₋₆ alkyl, optionally substituted with one or more halogen selected from the group consisting of F; and Cl;

R^{a}, R^{c} are independently selected from the group consisting of H; F; Cl; and CN;

R^{b}, A are independently selected from the group consisting of H; F; Cl; CN; and A¹, provided that
(a) one of R^{b}, A is A¹; and
(b) when A is A¹ and n is 1 and R⁶, R⁷ jointly form an oxo (═O), X is other than —C(R¹⁶R^{a})—O—; —C(R¹⁶R^{a})—NR^{c}—; or —C(R¹⁶R^{a})—CR¹⁷R^{c}—; and
(c) when A is A¹ and n is 2 and R⁶, R⁷ jointly form an oxo (═O), X is other than C(R¹⁶R^{c})—;

optionally R^{c} is selected from the group consisting of —O—C₁₋₆ alkyl; —O—C₃₋₇ cycloalkyl; —S—C₁₋₆ alkyl; —S—C₃₋₇ cycloalkyl; —N(R¹⁸)—C₁₋₆ alkyl; and —N(R¹⁸)—C₃₋₇ cycloalkyl, wherein each C₁₋₆ alkyl and C₃₋₇ cycloalkyl is optionally substituted with one or more R^{18a}, wherein R^{18a} is independently selected from the group consisting of halogen; C₁₋₆ alkyl; and C₃₋₇ cycloalkyl, provided that n is 1;

R¹³ is independently selected from the group consisting of H; C₁₋₆ alkyl;

optionally a pair of R^{a}, R^{b}, R^{c} selected from the group consisting of R^{a}/R^{c}; and R^{b}/R^{c} forms a ring Z¹; provided that R^{b}/R^{c} form a ring other than
(a) pyrimidine when R⁶, R⁷ jointly form an oxo (═O) and n is 1 and X is —C(R⁶R^{a})—C(R^{c})═;
(b) a ring selected from the group consisting of 1,2-diazole; and 1,2,4-triazole when R⁶, R⁷ jointly form an oxo (═O) and n is 1 and X is C(R¹⁶R^{a})—N(R^{c})—;

Z¹ is selected from the group consisting of Z²; and Z³;

Z² is selected from the group consisting of phenyl; naphthyl; and indenyl; wherein Z² is optionally substituted with one or more R¹⁹; wherein R¹⁹ is independently selected from the group consisting of halogen; CN; COOR²⁰; OR²⁰; C(O)N(R²OR^{20a}); S(O)₂N(R²OR^{20a}); C₁₋₆ alkyl; O—C₁₋₆ alkyl; S—C₁₋₆ alkyl; COO—C₁₋₆ alkyl; OC(O)—C₁₋₆ alkyl; C(O)N(R²⁰)—C₁₋₆ alkyl; S(O)₂N(R²″)—C₁₋₆ alkyl; S(O)N(R²⁰)—C₁₋₆ alkyl; S(O)₂—C₁₋₆ alkyl; S(O)—C₁₋₆ alkyl; N(R²⁰)S(O)₂—C₁₋₆ alkyl; and N(R²⁰)S(O)—C₁₋₆ alkyl; wherein each C₁₋₆ alkyl is optionally substituted with one or more halogen selected from the group consisting of F; and Cl;

Z³ is selected from the group consisting of C₃₋₇ cycloalkyl; indanyl; tetralinyl; decalinyl; heterocycle; and heterobicycle; wherein Z³ is optionally substituted with one or more R²¹, wherein R²¹ is independently selected from the group consisting of halogen; CN; OR²²; oxo (═O), where the ring is at least partially saturated; N(R²²R^{22a}); COOR²²; C(O)N(R²²R^{22a}); S(O)₂N(R²²R^{22a}); S(O)N(R²²R^{22a}); C₁₋₆ alkyl; O₁₋₆ alkyl; S—C₁₋₆ alkyl; N(R²²)—C₁₋₆ alkyl; COO—C₁₋₆ alkyl; OC(O)—C₁₋₆ alkyl; C(O)N(R²²)—C₁₋₆ alkyl; N(R²²)—C(O)—C₁₋₆ alkyl; S(O)₂N(R²²)—C₁₋₆ alkyl; S(O)N(R²²)—C₁₋₆ alkyl; S(O)₂—C₁₋₆ alkyl; S(O)—C₁₋₆ alkyl; N(R²²)S(O)₂—C₁₋₆ alkyl; and N(R²²)S(O)₃—C₁₋₆ alkyl; wherein each C₁₋₆ alkyl is optionally substituted with one or more halogen selected from the group consisting of F; and Cl;

optionally R²¹ is C(O)R²², provided that C(O)R²² is bound to a nitrogen, which is a ring atom of a heterocycle or heterobicycle;

R²⁰, R^{20a}, R²², R^{22a} are independently selected from the group consisting of H; C₁₋₆ alkyl; C₃₋₇ cycloalkyl; and —C₁₋₆ alkyl-C₃₋₇ cycloalkyl;

A¹ is selected from the group consisting of phenyl; heterocycle; heterobicycle; C₃₋₇ cycloalkyl, wherein A¹ is substituted with R²³ and wherein phenyl is optionally substituted with one R²⁴ and wherein heterocycle; heterobicycle; C₃₋₇ cycloalkyl are optionally substituted with one R²⁵;

R²⁴ is selected from the group consisting of halogen; CN; COOR²⁶; OC(O)R²⁶; OR²⁶; —C₁₋₆alkyl-OR²⁶; SR²⁶; C(O)N(R²⁶R²⁷); S(O)₂N(R²⁶R²⁷); S(O)N(R²⁶R²⁷); C₁₋₆ alkyl; N(R²⁶)S(O)₂R²⁷; and N(R²⁶)S(O)R²⁷; wherein each C₁₋₆ alkyl is optionally substituted with one or more halogen selected from the group consisting of F; and Cl;

R²⁵ is selected from the group consisting of halogen; CN; OR²⁶; —C₁₋₆alkyl-OR²⁶SR²⁶; oxo (═O), where the ring is at least partially saturated; N(R²⁶R²⁷); COOR²⁶; OC(O)R²⁶; C(O)N(R²⁶R²⁷); S(O)₂N(R²⁶R²⁷); S(O)N(R²⁶R²⁷); C₁₋₆ alkyl; N(R²⁶)C(O)R²⁷; S(O)₂R²⁶; S(O)R²⁶; N(R²⁶)S(O)₂R²⁷; and N(R²⁶)S(O)R²⁷; wherein each C₁₋₆ alkyl is optionally substituted with one or more halogen selected from the group consisting of F; and Cl;

optionally R²⁵ is C(O)R²⁶, provided that C(O)R²⁶ is bound to a nitrogen, which is a ring atom of a heterocycle or heterobicycle;

R²⁶, R²⁷ are independently selected from the group consisting of H; C₁₋₆ alkyl; C₃₋₇ cycloalkyl; and —C₁₋₆ alkyl-C₃₋₇ cycloalkyl; wherein each C₁₋₆ alkyl is optionally substituted with one or more halogen selected from the group consisting of F; and Cl;

R²³ is selected from the group consisting of F; Cl; oxo (═O), where the ring is at least partially saturated; OR^{23a}; N(R^{23a}R^{23b}); C₁₋₆ alkyl, optionally substituted with one or more R²⁸; and —(C(R²⁹R^{29a}))ₘ—W—(C(R³⁰R^{30a}))ₒ-T;

R²⁸ is selected from the group consisting of halogen; CN; COOR³¹; OC(O)R³¹; OR³¹; SR³¹; C(O)N(R³¹R³²); S(O)₂N(R³¹R³²); S(O)N(R³¹R³²); N(R³¹)S(O)₂R³² and N(R³¹)S(O)R³²;

R²⁹, R^{29a}, R³⁰, R^{30a} are independently selected from the group consisting of H; F; and R³³;

optionally one or both pairs of R²⁹, R^{29a}, R³⁰, R^{30a} independently selected from the group consisting of R²⁹/R^{29a}; and R³⁰/R^{30a} form a C₃₋₇ cycloalkyl ring, which is optionally substituted with one or more of R^{33b}, wherein R^{33b} is independently selected from the group consisting of F; Cl; and OH;

R^{23a}, R^{23b}, R³¹, R³² are independently selected from the group consisting of H; C₁₋₆ alkyl; C₃₋₇ cycloalkyl; and —C₁₋₆ alkyl-C₃₋₇ cycloalkyl; wherein each C₁₋₆ alkyl is optionally substituted with one or more halogen selected from the group consisting of F; and Cl;

R³³ is selected from the group consisting of C₁₋₆ alkyl; C₃₋₇ cycloalkyl; and —C₁₋₆ alkyl-C₃₋₇ cycloalkyl, wherein R³³ is optionally substituted with one or more R^{33a}, wherein R³³, is independently selected from the group consisting of F; Cl; and OH;

m, o are independently selected from the group consisting of 0; 1; 2; and 3;

W is selected from the group consisting of a covalent bond; —O—; —S—; —S(O₂)—; —S(O)—; —N(R³⁴)—; —N(R³⁴)C(O)—; —C(O)N(R³⁴)—; —OC(O)—; —C(O)O—; —S(O₂)N(R³⁴)—; —N(R³⁴)S(O)₂—; S(O)N(R³⁴)—; and —N(R³⁴)S(O)—;

R³⁴ is selected from the group consisting of H; C₁₋₆ alkyl; C₃₋₇ cycloalkyl; and —C₁₋₆ alkyl-C₃₋₇ cycloalkyl; wherein each C₁₋₆ alkyl is optionally substituted with one or more halogen selected from the group consisting of F; and Cl;

T is selected from the group consisting of H; T¹; and T²;

T¹ is selected from the group consisting of phenyl; naphthyl; and indenyl; wherein T¹ is optionally substituted with one or more R³⁵; wherein R³⁵ is independently selected from the group consisting of halogen; CN; COOR³⁷; OC(O)R³⁷; OR³⁷; —C₁₋₆alkyl-OR³⁷; SR³⁷; S(O)R³⁷; S(O)₂R³⁷; C(O)N(R³⁷R³⁸); S(O)₂N(R³⁷R³⁸); S(O)N(R³⁷R³⁸); C₁₋₆ alkyl; N(R³⁷)S(O)₂R³⁸; and N(R³⁷)S(O)R³⁸; wherein each C₁₋₆ alkyl is optionally substituted with one or more halogen selected from the group consisting of F; and Cl;

T² is selected from the group consisting of C₃₋₇ cycloalkyl; indanyl; tetralinyl; decalinyl; heterocycle; and heterobicycle; wherein T² is optionally substituted with one or more R³⁶, wherein R³⁶ is independently selected from the group consisting of halogen; CN; OR³⁷; —C₁₋₆alkyl-OR³⁷SR³⁷; oxo (═O), where the ring is at least partially saturated; N(R³⁷R³⁸); COOR³⁷; OC(O)R³⁸; C(O)N(R³⁷R³⁸); S(O)₂N(R³⁷R³⁸); S(O)N(R³⁷R³⁸); C₁₋₆ alkyl; N(R³⁷)C(O)R³⁸; S(O)₂R³⁷; S(O)R³⁸; N(R³⁷)S(O)₂R³⁸; and N(R³⁷)S(O)R³⁸; wherein each C₁₋₆ alkyl is optionally substituted with one or more halogen selected from the group consisting of F; and Cl;

optionally R³⁶ is C(O)R³⁷, provided that C(O)R³⁷ is bound to a nitrogen, which is a ring atom of a heterocycle or heterobicycle; R³⁷, R³⁸ are independently selected from the group consisting of H; C₁₋₆ alkyl; C₃₋₇ cycloalkyl; and —C₁₋₆ alkyl-C₃₋₇ cycloalkyl; wherein each C₁₋₆ alkyl is optionally substituted with one or more halogen selected from the group consisting of F; and Cl.

Within the meaning of the present invention the terms are used as follows:

In case a variable or substituent can be selected from a group of different variants and such variable or substituent occurs more than once the respective variants can be the same or different.

“Alkyl” means a straight-chain or branched carbon chain that may contain double or triple bonds. It is generally preferred that alkyl doesn't contain double or triple bonds. “C₁₋₄ Alkyl” means an alkyl chain having 1-4 carbon atoms, e.g. at the end of a molecule methyl, ethyl, —CH═CH₂, —C≡CH, n-propyl, isopropyl, —CH═CH—CH₃, —CH₂—CH═CH₂, n-butyl, isobutyl, —CH═CH—CH₂—CH₃, —CH═CH—CH═CH₂, sec-butyl tert-butyl or amid, e.g. —CH₂—, —CH₂—CH₂—, —CH═CH—, —CH(CH₃)—, —C(CH₂)—, —CH₂—CH₂—CH₂—, —CH(C₂H₅)—, —CH(CH₃)₂—.

“C₁₋₆ Alkyl” means an alkyl chain having 1-6 carbon atoms, e.g. C₁₋₆ alkyl, methyl, ethyl, —CH═CH₂, —CO—CH, n-propyl, isopropyl, —CH═CH—CH₃, —CH₂—CH═CH₂, n-butyl, isobutyl, —CH═CH—CH₂—CH₃, —CH═CH—CH═CH₂, sec-butyl tert-butyl, n-pentane, n-hexane, or amid, e.g. —CH₂—, —CH₂—CH₂—, —CH═CH—, —CH(CH₃)—, —C(CH₂)—, —CH₂—CH₂—CH₂—, —CH(C₂H₅)—, —CH(CH₃)₂—. Each hydrogen of a C₁₋₆ alkyl carbon may be replaced by a substituent.

“C₃₋₇ Cycloalkyl” or “C₃₋₇ Cycloalkyl ring” means a cyclic alkyl chain having 3-7 carbon atoms, e.g. cyclopropyl, cyclobutyl, cyclopentyl, cyclohexyl, cyclohexenyl, cycloheptyl. Each hydrogen of a cycloalkyl carbon may be replaced by a substituent.

“Halogen” means fluoro, chloro, bromo or iodo. It is generally preferred that halogen is fluoro or chloro.

“Heterocycle” means a cyclopentane, cyclohexane or cycloheptane ring that may contain up to the maximum number of double bonds (aromatic or non-aromatic ring which is fully, partially or un-saturated) wherein at least one carbon atom up to 4 carbon atoms are replaced by a heteroatom selected from the group consisting of sulphur (including —S(O)—, —S(O)₂—), oxygen and nitrogen (including ═N(O)—) and wherein the ring is linked to the rest of the molecule via a carbon or nitrogen atom. Examples for a heterocycle are furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulpholane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, azepine or homopiperazine. “Heterocycle” means also azetidine.

“Heterobicycle” means a heterocycle which is condensed with phenyl or an additional heterocycle to form a bicyclic ring system. “Condensed” to form a bicyclic ring means that two rings are attached to each other by sharing two ring atoms. Examples for a heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, dihydroquinoline, isoquinoline, tetrahydroisoquinoline, dihydroisoquinoline, benzazepine, purine or pteridine.

A preferred stereochemistry of compounds or a pharmaceutically acceptable salt thereof according to the present invention is shown in formula (Ia): 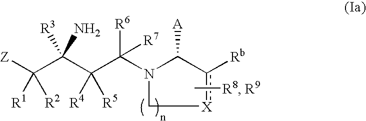
or a pharmaceutically acceptable salt thereof, wherein Z, R¹⁻⁹, n, A, X and R^{b} have the meaning as indicated above.

Preferred compounds of formula (I) or (Ia) are those compounds in which one or more of the residues contained therein have the meanings given below, with all combinations of preferred substituent definitions being a subject of the present invention. With respect to all preferred compounds of the formulas (I) or (Ia) the present invention also includes all tautomeric and stereoisomeric forms and mixtures thereof in all ratios, and their pharmaceutically acceptable salts.

In preferred embodiments of the present invention, the Z, R¹⁻⁹, n, A, X and R^{b} of the formula (I) or (Ia) independently have the following meaning. Hence, one or more of the substituents Z, R¹⁻⁹, n, A, X and R^{b} can have the preferred or more preferred meanings given below.

Preferably, Z is selected from the group consisting of phenyl; and heterocycle; and is optionally substituted with up to 3 R¹⁰, which are the same or different.

Preferably, R¹⁰ is selected from the group consisting of F; Cl; CN; and C₁₋₆ alkyl.

Preferably, R¹, R², R⁴, R⁵ are independently selected from the group consisting of H; F; and C₁₋₆ alkyl, optionally substituted with one or more F.

Preferably, R³ is H.

Preferably, R⁶ and R⁷ jointly form an oxo (═O) or R⁶ and R⁷ are independently selected from the group consisting of H; and C₁₋₆ alkyl, optionally substituted with one or more F.

Preferably, n is 0 and X is —CHR^{a}—CHR^{b}—.

Preferably, n is 1 and X is —CHR^{c}—.

Preferably, n is 1 and X is —C(R^{a})=CR^{c}— and R^{a}/R^{c} forms a ring Z¹.

Preferably, n is 1 and X is —CH(R^{a})—CR^{c}═ and R^{a}/R^{c} forms a phenyl.

Preferably, Z¹ is phenyl.

Preferably, R⁸, R⁹ are H.

Preferably, A is A¹.

Preferably, R^{a}, R^{b}, R^{c} are H.

In a further preferred embodiment, R^{a} and R^{c} are H and R^{b} is A¹.

Preferably, A¹ is heterocycle or heterobicycle.

More preferably, A¹ is heterocycle.

Particularly preferred, A¹ is selected from the group consisting of 1,2,4-oxadiazole; 1,2,4-triazole; 1,2,3-triazole; 1,2-diazole; oxazole, imidazole, and benzimidazole; wherein A¹ is substituted with R²³ and optionally substituted with R²⁵.

In a further more preferred embodiment, A¹ is selected from the group consisting of 1,3-diazole; 1,3,4-oxadiazole; piperidine; and piperazine, wherein A¹ is substituted with R²³ and optionally substituted with R²⁵.

Preferably, R²³ is (C(R²⁹R^{29a}))ₘ—W—(C(R³⁰R^{30a}))ₒ-T.

Preferably, R²⁵ is Cl.

Preferably, m and o are 0, 1 or 2. More preferred, m and o are 0.

R²⁹, R^{29a}, R³⁰ and R^{30a} are preferably C₁₋₆alkyl.

Preferably, W is a covalent bond.

In a further preferred embodiment, W is —O—.

More preferably, R²³ is selected from the group consisting of —C(CH₃)₃; —C(CH₃)₂—CH₂—O—CH₃; —C(CH₃)₂F; and T.

Preferably, T is H; or T is phenyl, optionally substituted with one or two R³⁵, which are the same or different; or T is selected from the group consisting of heterocycle; and C₃₋₇ cycloalkyl; wherein T is optionally substituted with one or two R³⁶, which are the same or different.

More preferred, T is selected from the group consisting of pyridine; azetidine; cyclopropyl; and cyclobutyl. Particularly preferred, T is substituted by halogen or C₁₋₆ alkyl which is substituted by with one or more fluoro.

In a further more preferred embodiment, T is selected from the group consisting of morpholine; pyrrolidine; pyrimidine; pyrazine; and oxetane; optionally substituted with R³⁵ or R³⁶.

Preferably, R³⁵, R³⁶ are independently selected from the group consisting of F; Cl; —S(O)₂—C₁₋₆alkyl; —S(O)₂NH₂; —S(O)₂—C₁₋₆ alkyl; —NH—S(O)₂—C₁₋₆ alkyl; and —N(C₁₋₆ alkyl)—S(O)₂—C₁₋₆ alkyl.

It is further preferred that R³⁶ is selected from the group consisting of OH; —C(O)C₁₋₆ alkyl; C₁₋₆ alkyl-O—C₁₋₆ alkyl; and C₁₋₆ alkyl optionally substituted with one or more halogen selected from the group consisting of F; and Cl.

Compounds of the formula (I) or (Ia) in which some or all of the above-mentioned groups have the preferred or more preferred meanings are also an object of the present invention.
Preferred embodiments of the compounds according to present invention are:

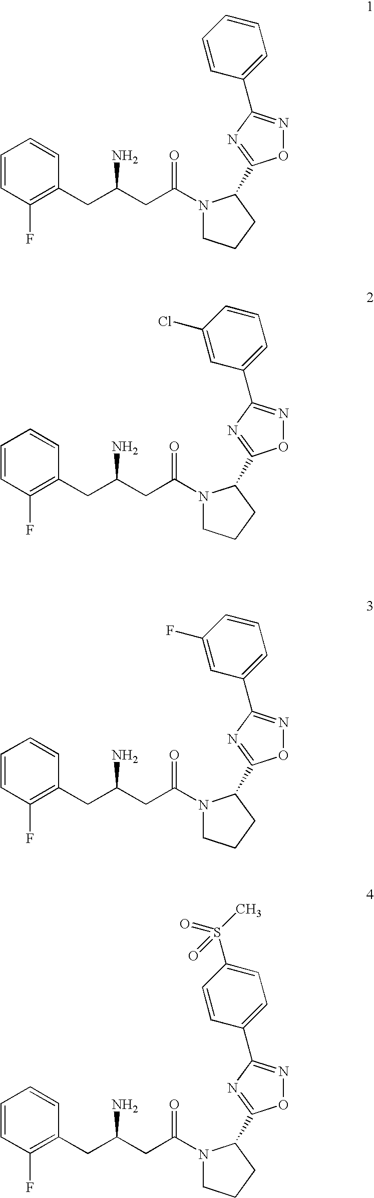

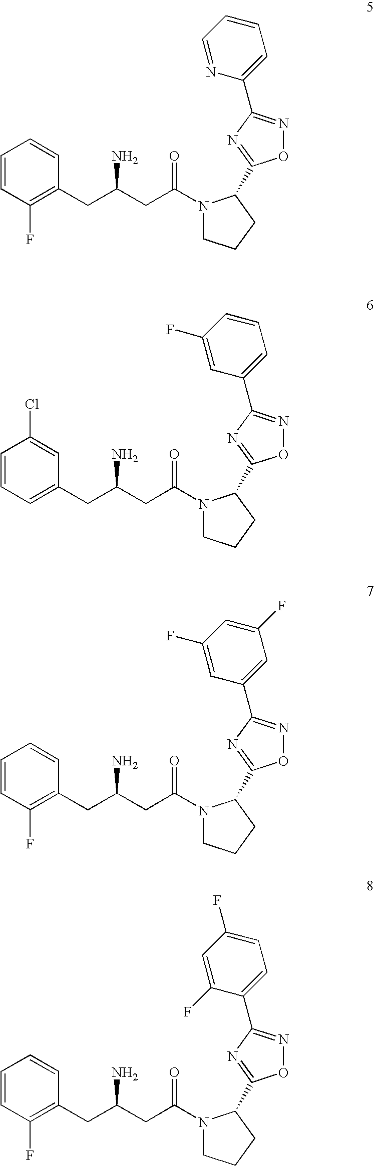

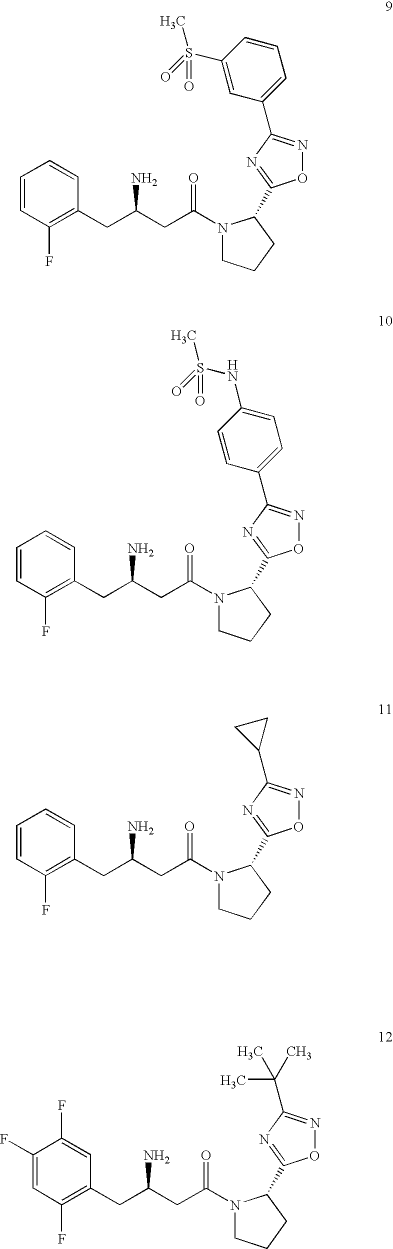

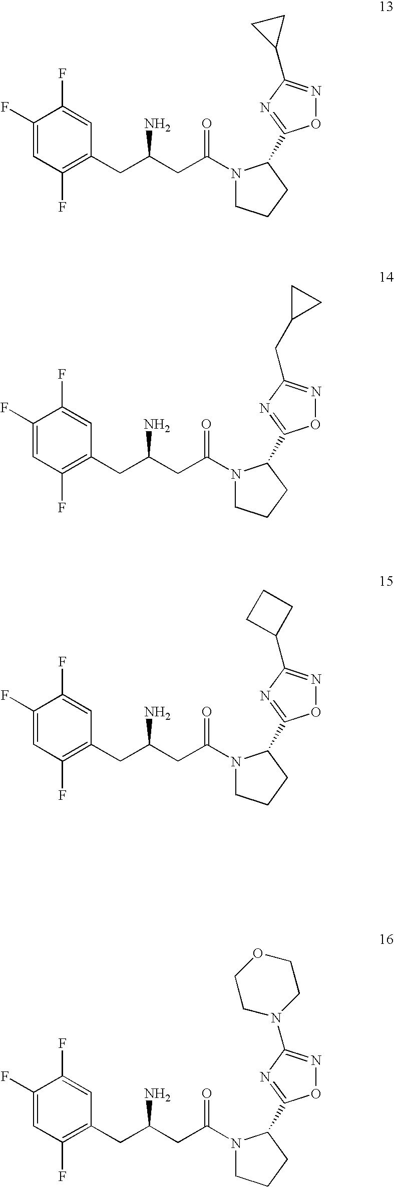

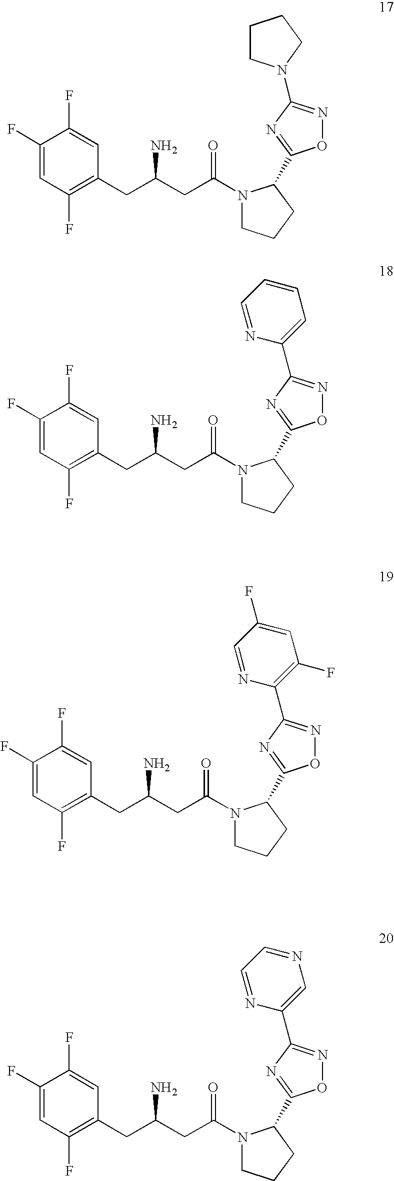

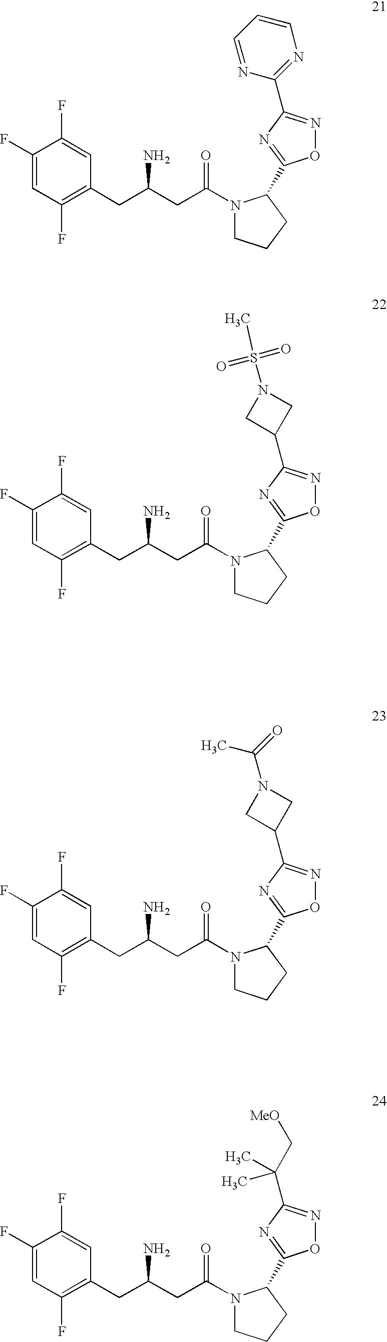

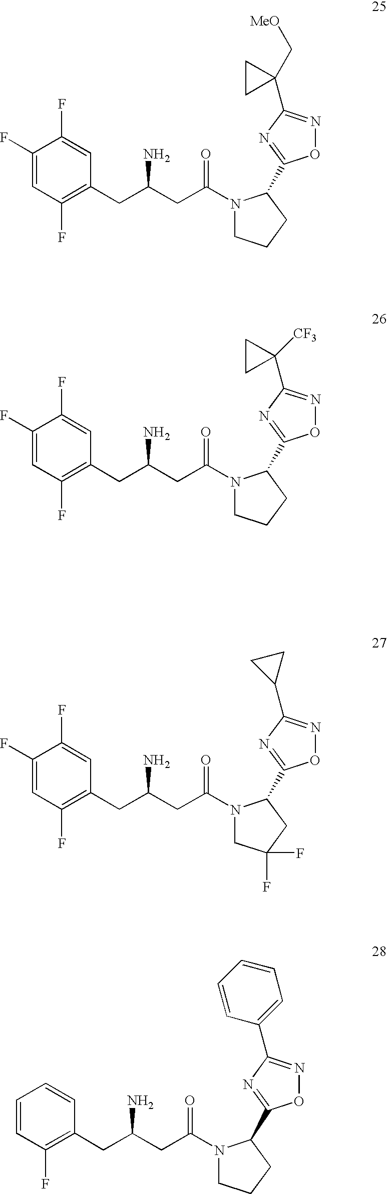

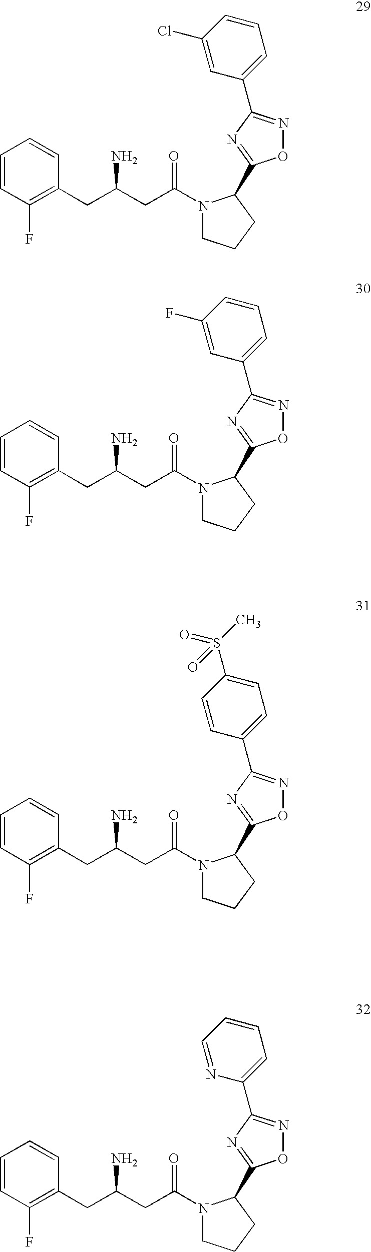

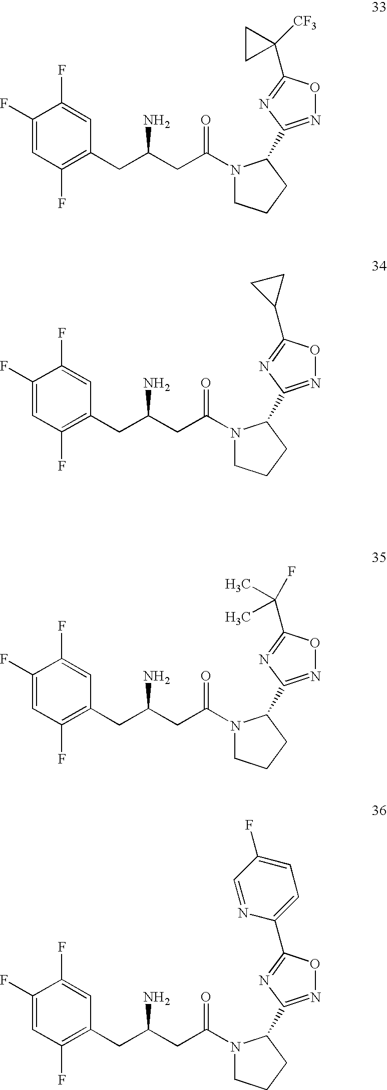

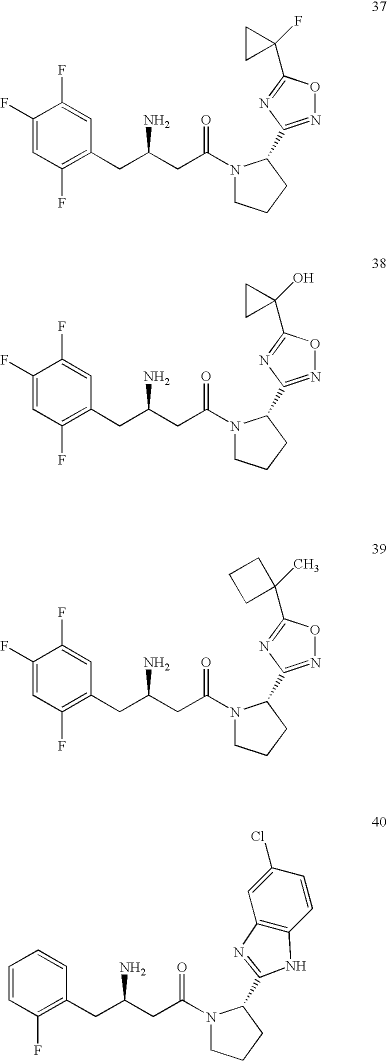

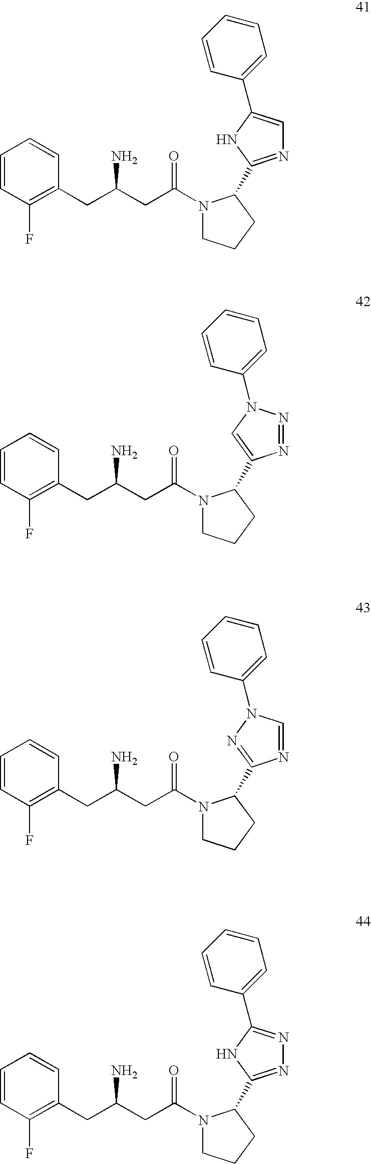

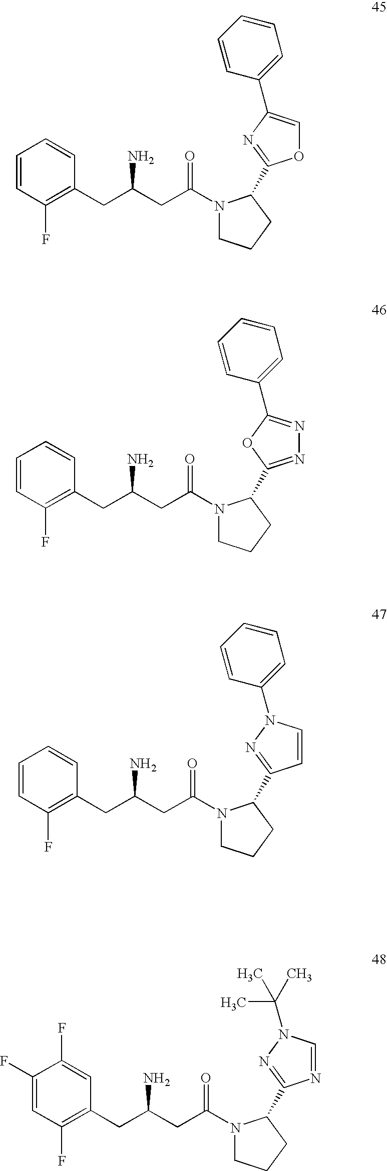

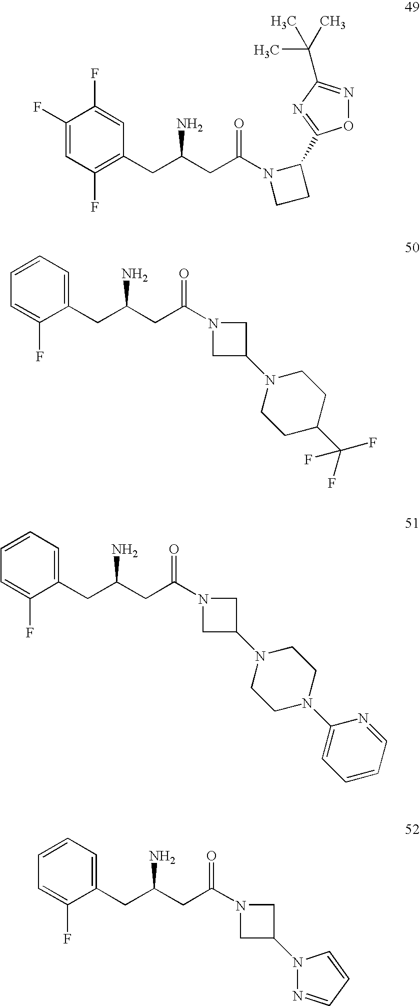

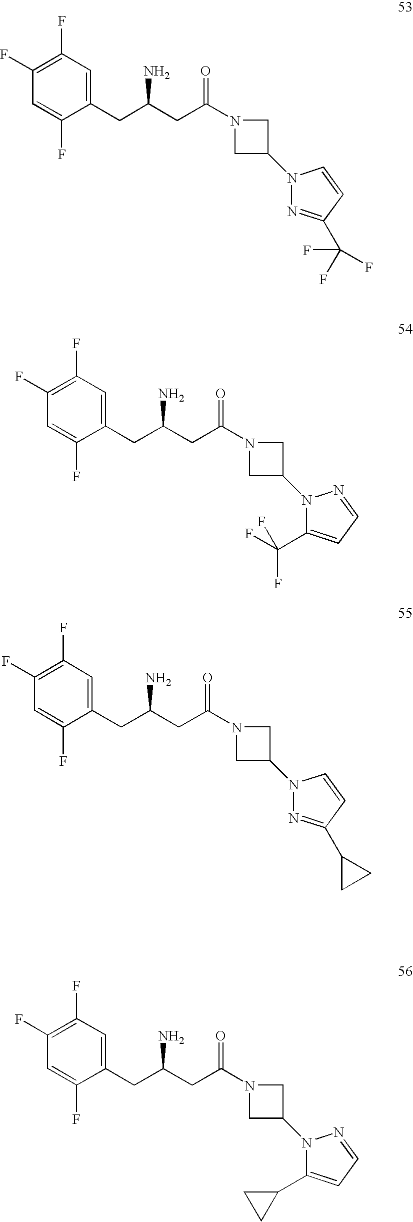

Furthermore, the present invention provides prodrug compounds of the compounds of the invention as described above.

“Prodrug compound” means a derivative that is converted into a compound according to the present invention by a reaction with an enzyme, gastric acid or the like under a physiological condition in the living body, e.g. by oxidation, reduction, hydrolysis or the like, each of which is carried out enzymatically. Examples of the prodrug are compounds, wherein the amino group in a compound of the present invention is acylated, alkylated or phosphorylated to form, e.g., eicosanoylamino, alanylamino, pivaloyloxymethylamino or wherein the hydroxyl group is acylated, alkylated, phosphorylated or converted into the borate, e.g. acetyloxy, palmitoyloxy, pivaloyloxy, succinyloxy, fumaryloxy, alanyloxy or wherein the carboxyl group is esterified or amidated. These compounds can be produced from compounds of the present invention according to well-known methods.

Metabolites of compounds of formula (I) or (Ia) are also within the scope of the present invention.

Where tautomerism, like e.g. keto-enol tautomerism, of compounds of general formula (I) or (Ia) or their prodrugs may occur, the individual forms, like e.g. the keto and enol form, are claimed separately and together as mixtures in any ratio. Same applies for stereoisomers, like e.g. enantiomers, cis/trans isomers, conformers and the like. If desired, isomers can be separated by methods well known in the art, e.g. by liquid chromatography. Same applies for enantiomers by using e.g. chiral stationary phases. Additionally, enantiomers may be isolated by converting them into diastereomers, i.e. coupling with an enantiomerically pure auxiliary compound, subsequent separation of the resulting diastereomers and cleavage of the auxiliary residue. Alternatively, any enantiomer of a compound of formula (I) or (Ia) may be obtained from stereoselective synthesis using optically pure starting materials.

In case the compounds according to formula (I) or (Ia) contain one or more acidic or basic groups, the invention also comprises their corresponding pharmaceutically or toxicologically acceptable salts, in particular their pharmaceutically utilizable salts. Thus, the compounds of the formula (I) or (Ia) which contain acidic groups can be present on these groups and can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids. Compounds of the formula (I) or (Ia) which contain one or more basic groups, i.e. groups which can be protonated, can be present and can be used according to the invention in the form of their addition salts with inorganic or organic acids. Examples for suitable acids include hydrogen chloride, hydrogen bromide, phosphoric acid, sulphuric acid, nitric acid, methanesulphonic acid, p-toluenesulphonic acid, naphthalenedisulphonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulphaminic acid, phenylpropionic acid, gluconic acid, ascorbic acid, isonicotinic acid, citric acid, adipic acid, and other acids known to the person skilled in the art. If the compounds of the formula (I) or (Ia) simultaneously contain acidic and basic groups in the molecule, the invention also includes, in addition to the salt forms mentioned, inner salts or betaines (zwitterions). The respective salts according to the formula (I) or (Ia) can be obtained by customary methods which are known to the person skilled in the art like, for example by contacting these with an organic or inorganic acid or base in a solvent or dispersant, or by anion exchange or cation exchange with other salts. The present invention also includes all salts of the compounds of the formula (I) or (Ia) which, owing to low physiological compatibility, are not directly suitable for use in pharmaceuticals but which can be used, for example, as intermediates for chemical reactions or for the preparation of pharmaceutically acceptable salts.

The present invention provides compounds of general formula (I) or (Ia) or their prodrugs as DPP-IV inhibitors. DPP-IV is a cell surface protein that has been implicated in a wide range of biological functions. It has a broad tissue distribution (intestine, kidney, liver, pancreas, placenta, thymus, spleen, epithelial cells, vascular endothelium, lymphoid and myeloid cells, serum), and distinct tissue and cell-type expression levels. DPP-IV is identical to the T cell activation marker CD26, and it can cleave a number of immunoregulatory, endocrine, and neurological peptides in vitro. This has suggested a potential role for this peptidase in a variety of disease processes.

DPP-IV related diseases are described in more detail in WO-A-03/181 under the paragraph “Utilities” which is herewith incorporated by reference.

Accordingly, the present invention provides compounds of formula (I) or (Ia) or their prodrugs or pharmaceutically acceptable salt thereof for use as a medicament.

Furthermore, the present invention provides the use of compounds of formula (I) or (Ia) or their prodrugs or a pharmaceutically acceptable salt thereof for the manufacture of a medicament for the treatment or prophylaxis of non-insulin dependent (Type II) diabetes mellitus; hyperglycemia; obesity; insulin resistance; lipid disorders; dyslipidemia; hyperlipidemia; hypertriglyceridemia; hypercholestrerolemia; low HDL; high LDL; atherosclerosis; growth hormone deficiency; diseases related to the immune response; HIV infection; neutropenia; neuronal disorders; tumor metastasis; benign prostatic hypertrophy; gingivitis; hypertension; osteoporosis; diseases related to sperm motility; low glucose tolerance; insulin resistance; ist sequelae; vascular restenosis; irritable bowel syndrome; inflammatory bowel disease; including Crohn's disease and ulcerative colitis; other inflammatory conditions; pancreatitis; abdominal obesity; neurodegenerative disease; retinopathy; nephropathy; neuropathy; Syndrome X; ovarian hyperandrogenism (polycystic ovarian syndrome; Type n diabetes; or growth hormone deficiency. Preferred is non-insulin dependent (Type II) diabetes mellitus and obesity.

The present invention provides pharmaceutical compositions comprising a compound of formula (I) or (Ia), or a prodrug compound thereof, or a pharmaceutically acceptable salt thereof as active ingredient together with a pharmaceutically acceptable carrier.

“Pharmaceutical composition” means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

A pharmaceutical composition of the present invention may additionally comprise one or more other compounds as active ingredients like one or more additional compounds of formula (I) or (Ia), or a prodrug compound or other DPP-IV inhibitors.

Other active ingredients are disclosed in WO-A-03/181 under the paragraph “Combination Therapy” which is herewith incorporated by reference.

Accordingly, other active ingredients may be insulin sensitizers; PPAR agonists; biguanides; protein tyrosinephosphatase-IB (PTP-1B) inhibitors; insulin and insulin mimetics; sulphonylureas and other insulin secretagogues; α-glucosidase inhibitors; glucagon receptor antagonists; GLP-1, GLP-1 mimetics, and GLP-1 receptor agonists; GIP, GIP mimetics, and GIP receptor agonists; PACAP, PACAP mimetics, and PACAP receptor 3 agonists; cholesterol lowering agents; HMG-CoA reductase inhibitors; sequestrants; nicotinyl alcohol; nicotinic acid or a salt thereof; PPAR^{a} agonists; PPARoly dual agonists; inhibitors of cholesterol absorption; acyl CoA: cholesterol acyltransferase inhibitors; anti-oxidants; PPARo agonists; antiobesity compounds; an ileal bile acid transporter inhibitor; or anti-inflammatory agents or pharmaceutically acceptable salts of these active compounds.

The term “pharmaceutically acceptable salts” refers to salts prepared from pharmaceutically acceptable non-toxic bases or acids, including inorganic bases or acids and organic bases or acids.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds of formula (I) or (Ia) can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavour.

Compounds of formula (I) or (Ia) may also be administered parenterally. Solutions or suspensions of these active compounds can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Any suitable route of administration may be employed for providing a mammal, especially a human, with an effective dose of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of formula (I) or (Ia) are administered orally.

The effective dosage of active ingredient employed may vary depending on the particular compound employed, the mode of administration, the condition being treated and the severity of the condition being treated. Such dosage may be ascertained readily by a person skilled in the art.

When treating or preventing diabetes mellitus and/or hyperglycemia or hypertriglyceridemia or other diseases for which compounds of Formula I are indicated, generally satisfactory results are obtained when the compounds of the present invention are administered at a daily dosage of from about 0.1 milligram to about 100 milligram per kilogram of animal body weight, preferably given as a single daily dose or in divided doses two to six times a day, or in sustained release form. For most large mammals, the total daily dosage is from about 1.0 milligrams to about 1000 milligrams, preferably from about 1 milligrams to about 50 milligrams. In the case of a 70 kg adult human, the total daily dose will generally be from about 7 milligrams to about 350 milligrams. This dosage regimen may be adjusted to provide the optimal therapeutic response.

Some abbreviations that may appear in this application are as follows.

Abbreviations

Designation

Boc (or boc) tert-Butoxycarbonyl
CDI 1,1′-Carbonyldiimidazole
DAST Diethylaminosulphor trifluoride
DCE Dichloroethane
DCM Dichloromethane
DDQ 2,3-Dichloro-5,6-dicyano-1,4-benzoquinone
Deoxofluor Bis-(2-methoxy-ethyl)-aminosulphor trifluoride
DIC 1,3-Di-iso-propylcarbodiimide
DIPEA Di-iso-propyl-ethylamine
DMF N,N-Dimethylformamide
EDC 1-Ethyl-3-(3-dimethylaminopropyl)carbodiimide hydrochloride
Et₃N Triethylamine
h hour(s)
HATU O-(7-Azabenzotriazol-1-yl)-N,N,N′, N′-tetramethyluronium hexafluorophosphate
HBTU O-(Benzotriazol-1-yl)-N,N,N′,N′-tetramethyluronium hexafluorophosphate
HOBt 1-Hydroxybenzotriazole
HPLC High pressure liquid chromatography
min minute(s)
PG (or pg) Protecting group
rt Retention time
TFA Trifluoroacetic acid

Available starting materials may be suitably N-protected amino acids (II), nitriles (III), aldehydes (IV) or alcohols (V).

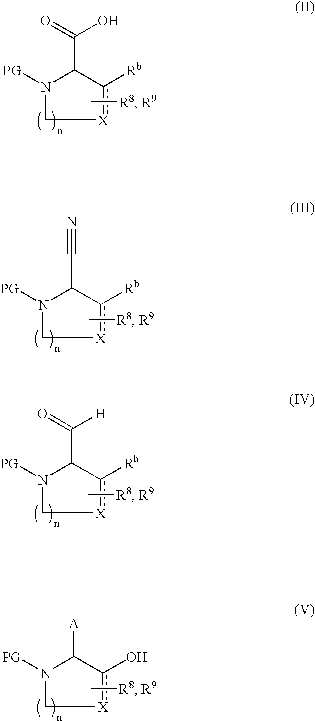

They may be purchased from commercially available sources such as ABCR, Array, Astatech, Sigma-Aldrich, Fluka or be synthesised by
(a) dehydration of the corresponding amide (Scheme A) 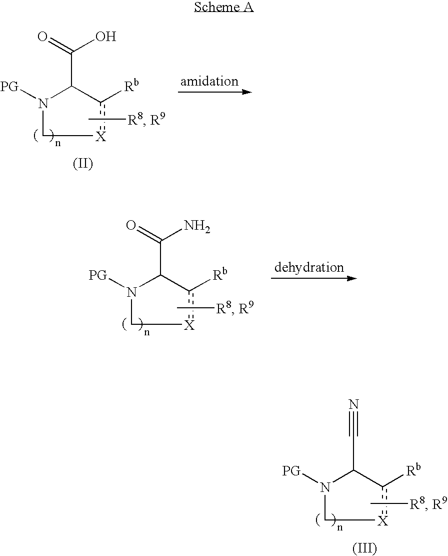
(b) reduction of the Weinreb amide of the carboxylic acid with metal hydride reagents e.g. lithium aluminumhydride (Scheme B).

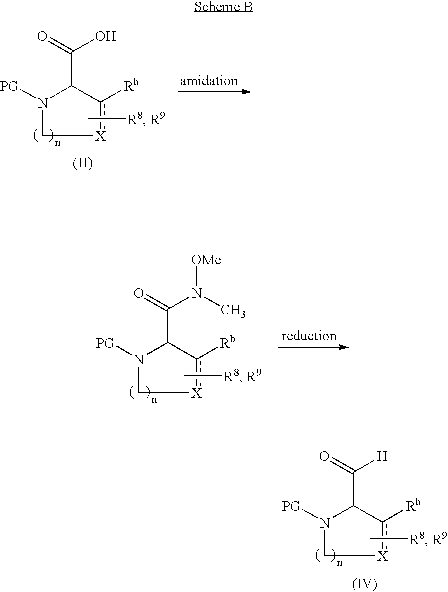

The functional groups of the N-protected amino acids may then be transformed into heteroaromatic ring systems by the following reaction sequences:

Reacting an amino acid of the general structure (II) with amidoximes and subsequent dehydration and deprotection can lead to the formation of 1,2,4-oxadiazole compounds of the general type (VI), as shown in Scheme C. According to F. Eloy, R. Lenaers, Chem. Rev. 1962, 62, 155, starting amidoximes can be prepared from nitrites by reaction with hydroxylamine.

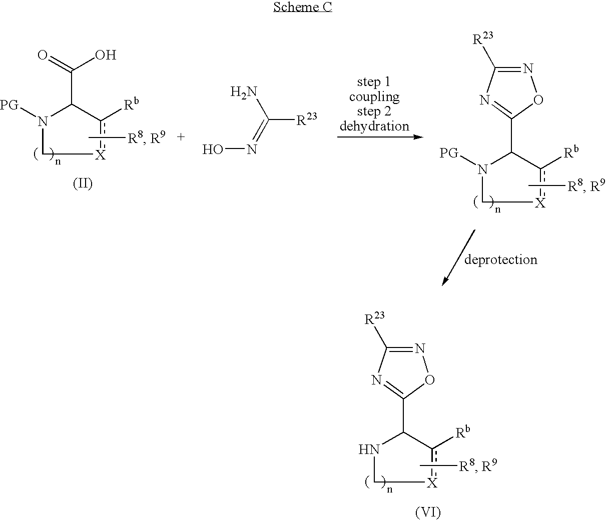

1,2,4-Oxadiazoles with a reversed substitution pattern can be prepared by using a similar reaction scheme starting from nitriles of the general type (III) as described in Scheme D. First, the nitriles are transformed into the amidoximes by reaction with hydroxylamine. The amidoximes are then coupled with carboxylic acids, and the resulting O-acyl amidoximes can be dehydrated to yield the desired 1,2,4-oxadiazoles of the general structure (VII).

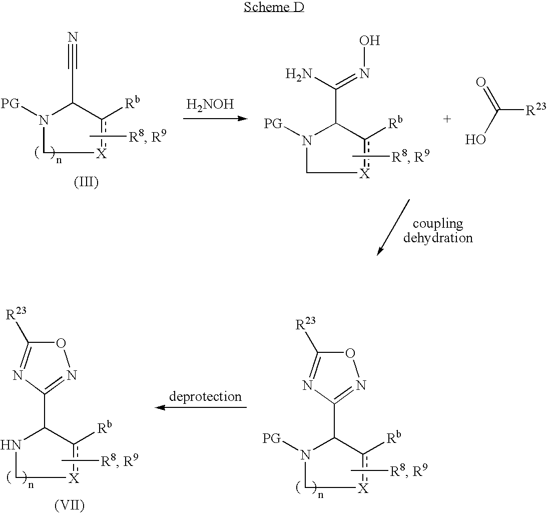

Pyrrazole containing amines of the general structure (VIII) may be synthesised according to M. Falorni, G. Giacomelli A. M. Spanedda, Tetrahedron: Asymmetry 1998, 9, 17, 3039-3046 as shown in Scheme E. Treating Weinreb amides, derived from amino acids of the general type (II), with trimethylsilyethynyl magnesium bromide may lead to the formation of propyn-3-one derivatives which can subsequently be treated with hydrazines to yield pyrazoles.

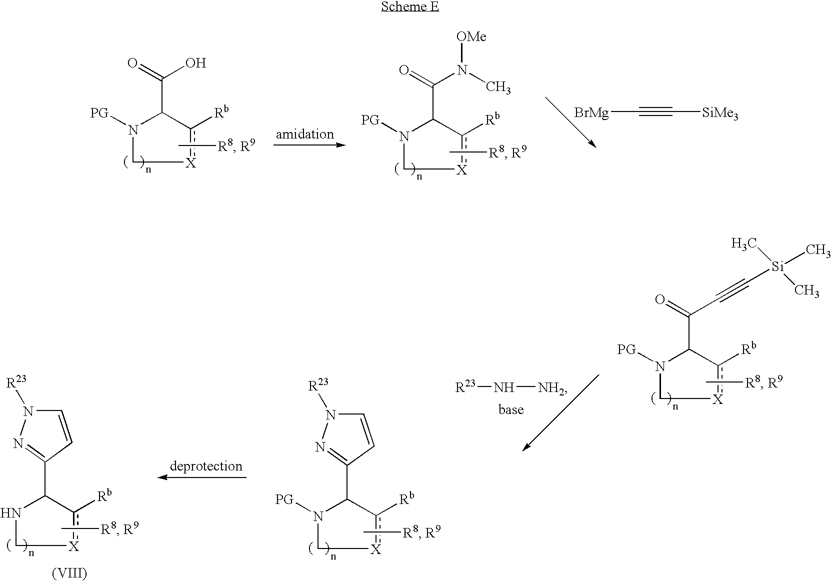

Scheme F depicts a general route towards 1,3,4-oxadiazoles of the structure (IX) as described, e.g. in C. T. Brain, J. M. Paul, Y. Loog, P. J. Oakley, Tetrahedron Lett. 1999, 40, 3275-3278. Amino acids of the general type (II) may be transformed into their acid chlorides that upon reaction with acylhydrazines may yield diacylhydrazines. Subsequent dehydration and deprotection can result in the formation of 1,3,4-oxadiazoles of the general type (IX).

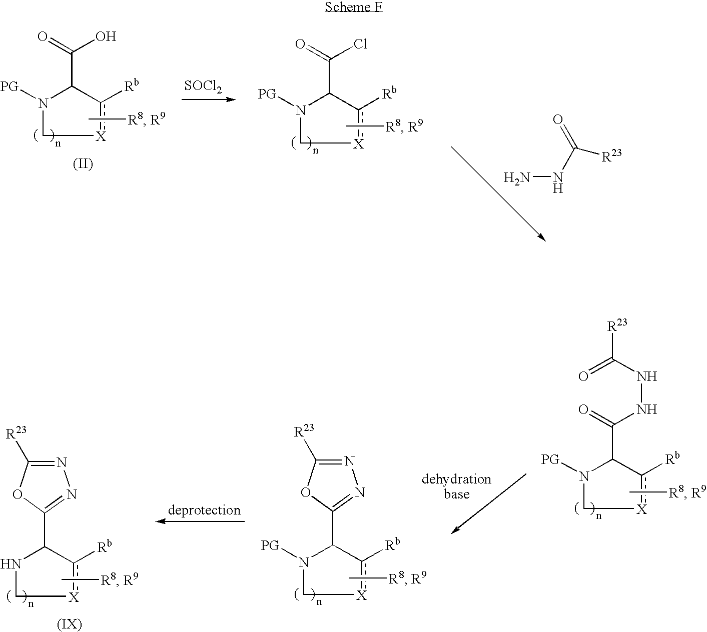

A typical synthesis of 1,3,4-triazoles is shown in Scheme G. Starting from amino acids of the general type (II) 1,3,4-triazoles can be prepared via amides that can be transformed into imidoyl chlorides by, e.g., phosphorousoxychloride. Treating these imidoyl chlorides with hydrazides under acidic conditions may lead to the formation of 1,3,4-triazoles that after final deprotection can result in structures of the general type (X).

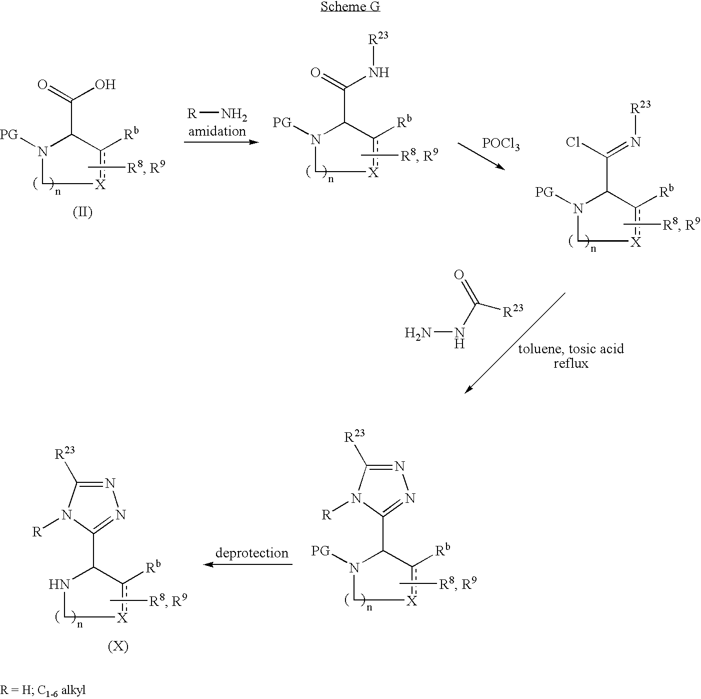

As shown in Scheme H benzimidazoles of the general type (XI) may be prepared from aldehydes of the type (IV) by reaction with diamino compounds, followed by oxidation and deprotection.

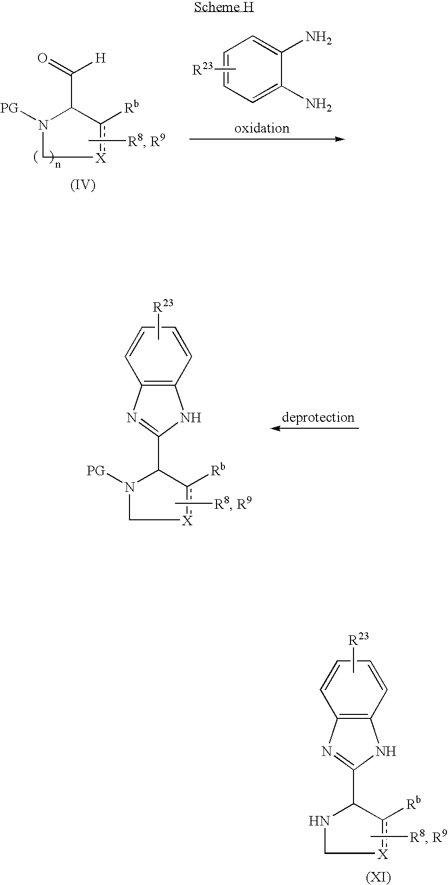

According to M.-O. Contour-Galcéra, L. Poitout, C. Moinet, B. Morgan, T. Gordon, P. Roubert, and C. Thurieau in Bioorg. Med. Chem. Lett. 2001, 11, 741-745 imidazoles of type (XII) can be prepared according to Scheme I. Condensing acid building blocks (II) with bromoacetyl fragments can yield ketoesters which upon treatment with ammoniumacetate yield protected imidazoles with the desired substitution pattern. Subsequent deprotection should furnish building blocks of the type (XII)

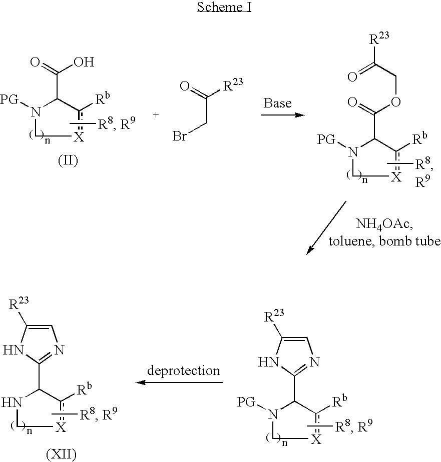

Oxazoles of type (XIII, Scheme J) can be prepared commencing from commercial amino alcohols and amino acids of the type (II). After an amide coupling step, dehydration can be achieved by several reactants, e.g., Burgess reagent, DAST or Deoxofluor as reported by A. J. Phillips, Y. Uto, P. Wipf, M. J. Reno, and D. R Williams in Org. Lett. 2000, 2, 8, 1165-1168. To yield the final oxazole, oxidation can be carried out with DDQ or nickel peroxide (NiO₂), an oxidant that was disclosed in this context by D. L. Evans, D. K. Minster, U. Jordis, S. M. Hecht, A. L. Mazzu, Jr., and A. I. Meyers in J. Org. Chem. 1979, 44, 4, 497-501, and by F. W. Eastwood, P. Perlmutter, Q. Yang in J. Chem. Soc., Perkin Trans. 1, 1997, 35-42. Finally, deprotection of the intermediate can yield oxazoles of the type (XIII).

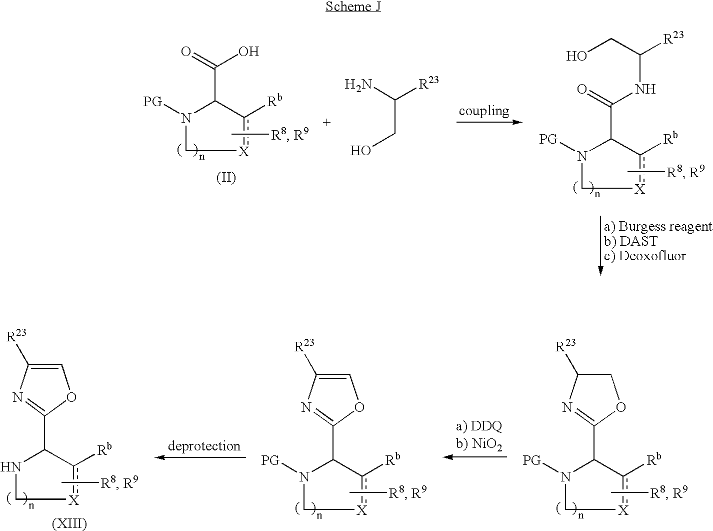

Starting form nitriles of the type (III) N-substituted 1,2,4-triazoles of the type (XIV) can be prepared as depicted in Scheme K. After addition of sodium alkoxide to nitrile (III), ring condensation can be achieved subsequently by reaction with hydrazines and triethylorthoformate to form the triazole ring, a method reported by H. J. Wadsworth, S. M. Jenkins, B. S. Orlek, F. Cassidy, M. S. G. Clark, F. Brown, G. J. Riley, D. Graves, J. Hawkins, and C. B. Naylor in J. Med. Chem. 1992, 35, 1280-1290. Finally, deprotection of the intermediate yields the desired triazole (XIV).

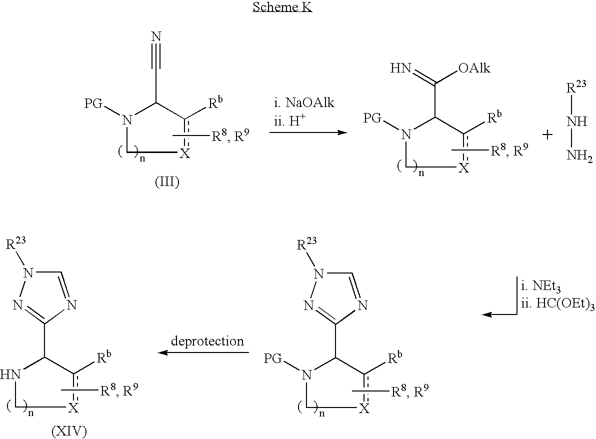

N-substituted 1,2,3-triazoles of the type (XV) can be prepared according to C. W. Tornøe, C. Christiansen, and M. Meldal in J. Org. Chem. 2002, 67, 3057-3064 or V. V. Rostovtsev, L. G. Green, V. V. Fokin, and K. B. Sharpless in Angew. Chem. 2002, 114, 2708-2711 by a dipolar cycloaddition reaction as shown in Scheme L. Commencing from aldehydes of the type (IV), acetylene intermediates can be prepared either via the Corey-Fuchs reaction sequence as examplified by D. S. Garvey, et al. in J. Med. Chem. 1994, 37, 1055-1059 or by implementation of a Seyferth-Gilbert phosphonate reagent as reported by G. J. Roth, et al. in Synthesis, 2004, 1, 59-62. Reaction with azides, freshly prepared by diazotransfer as disclosed by Q. Liu and Y. Tor in Org. Lett. 2003, 5, 14, 2571-2572, can yield N-substituted 1,2,3-triazoles. After a final deprotection step the desired building blocks of the type (XV) can be obtained.

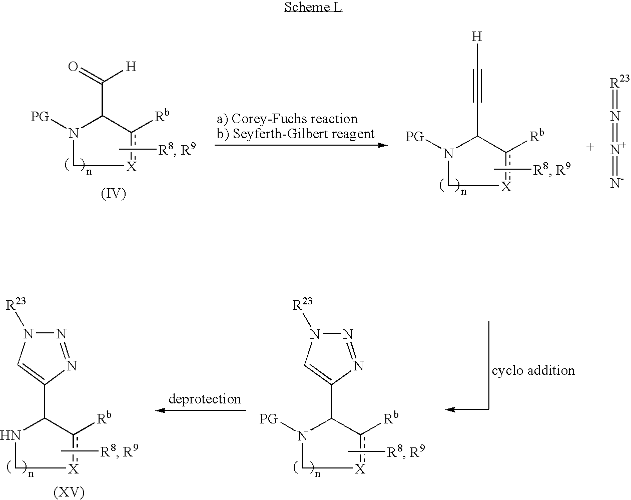

Other heterocyclic compounds may be prepared according to T. Eicher, S. Hauptmann, The Chemistry of Heterocycles, Ed. Wiley-VCH, Weinheim, 2003, or the literature cited therein.

Building blocks of the type (XVI) can be prepared from commercial alcohols (V) by a nucleophilic substitution reaction. Therefore the alcohol function has to be transformed into a suitable leaving group, e.g., halogene, mesylate or tosylate, that can be substituted under basic reaction conditions with a nucleophilic heterocycle. As such N-unsubstituted pyrrolidines, piperidines, piperazines, imidazoles, pyrazoles, triazoles can be employed in this reaction. Examples of this reaction can be found in Chem. Pharm. Bull. 1974, 22, 1490, U.S. Pat. No. 3,929,765 and WO 99/19297. Final deprotection yields the desired building block (XVI).

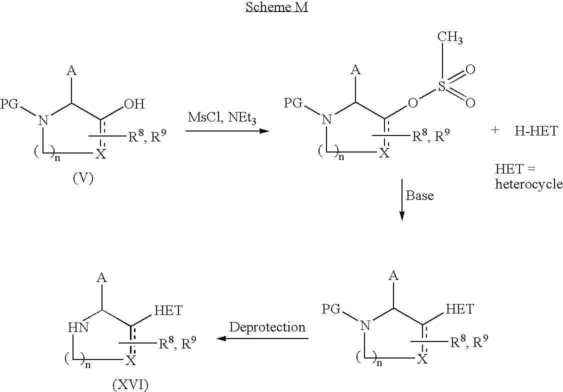

Enantiomerically pure beta amino acids having the formula (XVII) 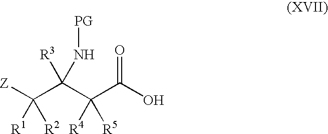
may be commercially available, known in the literature or may be conveniently synthesized using one of the methods already published and reviewed in e.g., Cole, Tetrahedron 1994, 32, 9517, Juaristi et al., Aldrichimica Acta 1994, 27, 3, or Juaristi, Enantioselective Synthesis of β-Amino Acids, Ed. Wiley-VCH, New York, 1997. In particular, 3-amino-4-(2,4,5-trifluoro-phenyl)-butyric acid may be synthesized by a variety of methods as reported in the patent applications WO 2004069162, WO 2004064778, WO 2004037169, WO 2004032836 and in the articles J. Am. Chem. Soc. 2004, 126, 3048 and J. Am. Chem. Soc. 2004, 126, 9918.

Enantiomerically pure beta amino aldehydes having the formula (XVIII) 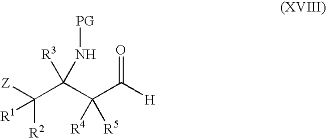
may be commercially available or synthesised by one skilled in the art using as starting material the conveniently substituted acids e.g. through direct reduction with di-iso-butylaluminium hydride or through formation of the Weinreb amide and further reduction with lithium aluminiumhydride, as depicted in Scheme N.

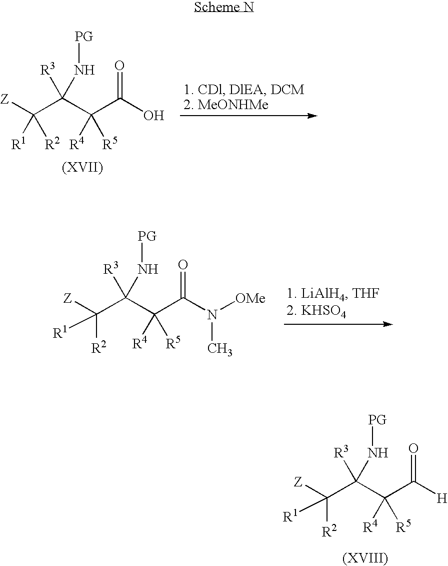

Unless otherwise noted, all non-aqueous reactions were carried out under argon atmosphere with commercial solvents. Compounds were purified using flash column chromatography using Merck silica gel 60 (230-400 mesh) or reverse phase preparative HPLC using a Reprosil-Pur ODS3, 5 μm, 20×125 mm column with Shimadzu LC8A-Pump and SPD-10Avp UV/V is diode array detector. The ¹H-NMR spectra were recorded on a Varian VXR-S (300 MHz for ¹H-NMR) using CDCl₃ or d₆-dimethylsulphoxide as solvent; chemical shifts are reported in ppm relative to tetramethylsilane. Analytical LC/MS was performed using Reprosil-Pur ODS3, 5 μM, 1×60 mm columns with a linear gradient from 5% to 95% acetonitrile in water (0.1% TFA or formic acid) at a flow rate of 250 μl/min; retention times are given in minutes. Methods are: (I) runs on a LC10Advp-Pump (Shimadzu) with SPD-M10Avp UVN is diode array detector and QP2010 MS-detector in ESI+ modus with UV-detection at 214, 254 and 275 nm, 5 min. linear gradient; (II) idem but 10 min. linear gradient. Analytical LC/MS was also performed using a XTerra MS C18, 3.5 μM, 2.1×100 mm columns from Waters in acetonitrile, water (0.1% formic acid) mixtures with a 15 min linear gradient (III) from 1% to 30% acetonitrile or (IV) from 10% to 60% acetonitrile at a flow rate of 400 μl/min; retention times are given in minutes.

General Procedures for Making Compounds of the Invention

In general, compounds having the structure (I) 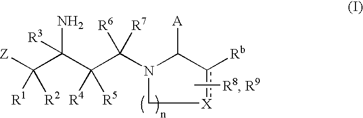
wherein the variables have the above described meanings, may be prepared by

(a) standard peptide coupling reactions. For example, it may be possible to use 1-ethyl-3-(3-dimethylaminopropyl)carbodiimide hydrochloride (EDC) in combination with 1-hydroxybenzotriazole (HOBt) and a base (triethylamine or diisopropylethylamine) or O-(7-azabenzotriazol-1-yl)-N,N,N′,N′-tetramethyluronium hexafluorophosphate (HATU) in the presence of a base, in solvents such as methylene chloride or N,N-dimethylformamide. Scheme O outlines a procedure for using the amines formed according to Schemes C through M to synthesize compounds that are embodiments of the invention.

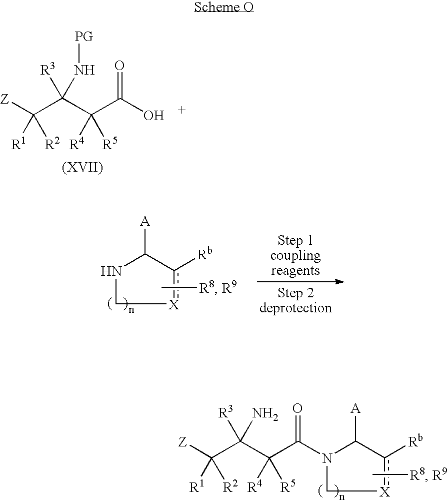

The protective group may be removed with, for example, diethylamine in dichloromethane in the case of 9-fluorenylmethoxycarbonyl or using acidic conditions (such as trifluoroacetic acid in dichloromethane or hydrochloric acid in dioxane) in the case of tert-butoxycarbonyl, as described in Protective Groups in Organic Synthesis 3^{rd} ed., Ed. Wiley-VCH, New York; 1999.

(b) reduction of suitable amides (e.g. with lithium aluminiumhydride in tetrahydrofuran) Scheme P outlines a procedure for using the amides formed according to Scheme O to synthesize compounds that are embodiments of the invention.

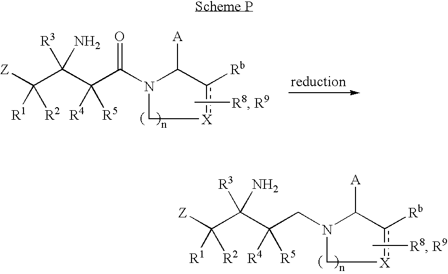

(c) reductive amination reducing the imine formed from a conveniently substituted aldehyde and an amine (e.g. in acidic medium with a triacetoxyborohydride salt or sodium borohydride in dichloromethane, methanol or ethanol). Scheme Q outlines a procedure for reducing the imines obtained by reacting suitable amines (e.g., prepared according to Schemes C to M) and aldehydes with formula (XVIII) or ketones.

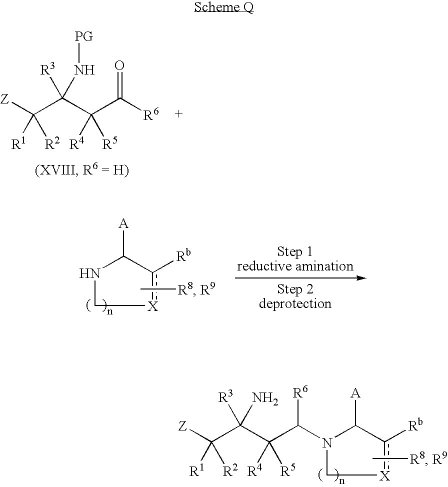

(d) the Kulnikovic reaction with an alkylmagnesium halide in the presence of titanium tetraisopropoxide in aprotic solvents such as tetrahydrofuran. Scheme R outlines a procedure for the use of the Kulnikovic reaction with amides prepared according to Scheme O.

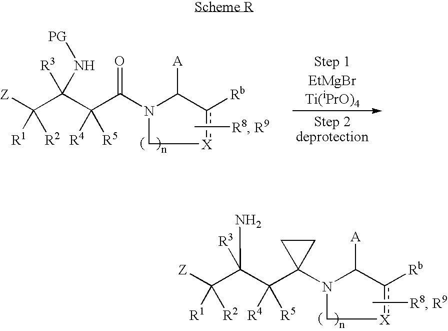

For the purification of intermediates or end products, flash chromatography on silica gel may be suitable for the free amines whereas the use of preparative HPLC leads to the isolation of the corresponding trifluoroacetic acid or formate salts. Compounds may be prepared by other means however, and the suggested starting materials and procedures described below are exemplary only and should not be considered as limiting the scope of the invention.

### EXAMPLES

The following examples are provided so that the invention might be more fully understood. These examples are illustrative only and should not be construed as limiting the invention in any way.

Preparations

### Example 1

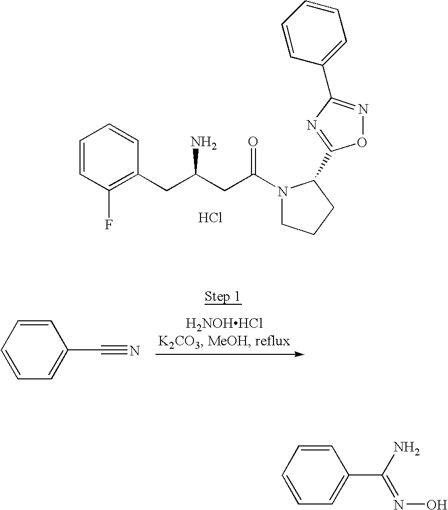

### N-Hydroxy-benzamidine

10.3 g (0.10 mol, 1.00 eq) of benzonitrile are dissolved in 40 mL of methanol, 20.7 g (0.15 mol, 1.50 eq) of powdered potassium carbonate is added and 13.9 g (0.20 mol, 2.00 eq) of hydroxylamine hydrochloride dissolved in 120 mL of methanol is added subsequently in small portions. The reaction mixture is refluxed for 5 h then the solvent is evaporated and the residue is taken up in a 1:4 mixture of water and chloroform. The organic layer is separated, washed twice with water, dried with magnesium sulphate, filtered and evaporated under reduced pressure. The residue is recrystallized from ether/hexane to afford the desired product.

Mp.: 77° C.-79° C.

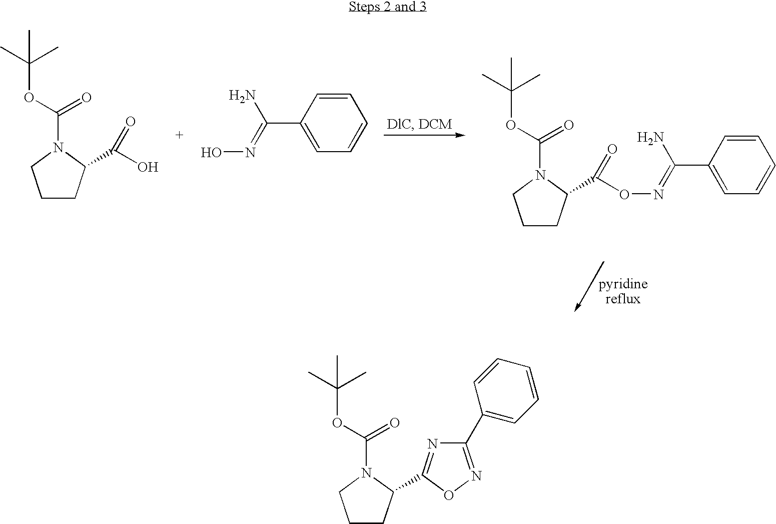

### 2-(3-Phenyl-[1,2,4]oxadiazol-5-yl)-(S)-pyrrolidine-1-carboxylic acid tert-butyl ester

To a solution of 5.75 g (42.2 mmol, 1.00 eq) N-hydroxy-benzamidine, 10.0 g (46.5 mmole, 1.10 eq) (S)-pyrrolidine-1,2-dicarboxylic acid 1-tert-butyl ester in 60 mL of dichloromethane, 6.44 g (51.0 mmol, 1.20 eq) of 1,3-di-iso-propylcarbodiimide is added and the mixture is stirred at room temperature. After 12 h the mixture is evaporated under reduced pressure and the residue is dissolved in 60 mL of pyridine and refluxed for 10 h. Then the pyridine is evaporated and the residue is taken up in a 2:1 mixture of dichloromethane and water. The aqueous phase is extracted with dichloromethane, and the combined organic layers are washed with 3% hydrochloric acid solution, saturated sodium bicarbonate solution, water and brine, dried with magnesium sulphate, filtered and evaporated under reduced pressure. The residue is subjected to column chromatography (silica gel, eluent: cyclo-hexane-ethyl acetate=60:40) to afford the title compound.

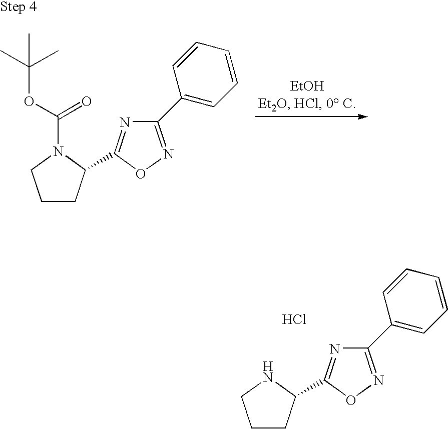

### 3-Phenyl-5-(S)-pyrrolidin-2-yl-[1,2,4]oxadiazole hydrochloride

The intermediate from step 3 is dissolved in 40 mL of ethanol and cooled to 0° C. Then 40 mL of diethyl ether saturated with gaseous hydrochloric acid is added and the reaction mixture is allowed to warm to room temperature. After complete conversion of the starting material, as monitored by TLC (silica gel, eluent: dichloroethane-ethanol 4:1), the solvents are evaporated. The residue is taken up in diethyl ether and the precipitate is filtered off, washed with diethyl ether and dried under reduced pressure to yield the title compound.

¹H-NMR (300 MHz, CDCl₃) δ=2.65-2.15 (m, 4H, 2×CH₂), 3.64 (m, 2H, CH₂), 5.2 (m, 1H, CH), 7.4-7.55 (m, 3H, aryl-H), 8.04 (m, 2H, aryl-H), 10.24 (s, 1H, NH), 11.18 (s, 1H, NH).

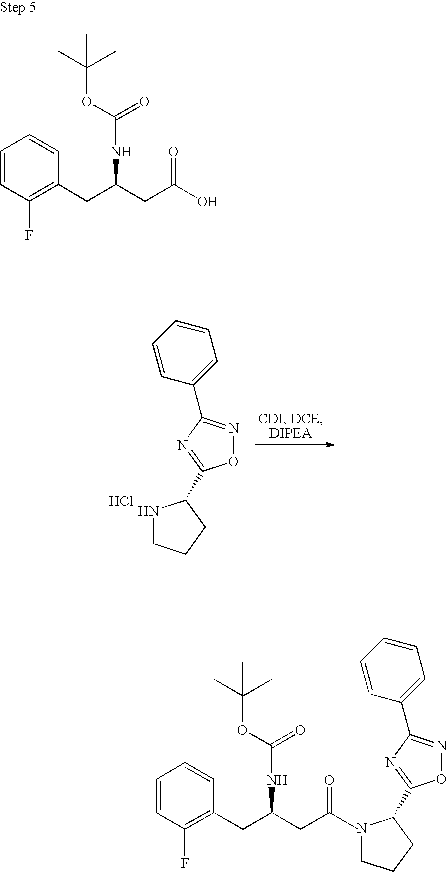

### (R)-{1-(2-Fluoro-benzyl)-3-oxo-3-[2-(3-phenyl-[1,2,4]oxadiazol-5-yl)-(S)-pyrrolidin-1-yl]-propyl}-carbamic acid tert-butyl ester

A mixture of 125 mg (0.42 mmol, 1.00 eq) of (R)-3-tert-butoxycarbonylamino-4-(2-fluoro-phenyl)-butyric acid and 71.4 mg (0.44 mmol, 1.05 eq) of 1,1′-carbonyl-diimidazole in 5.00 mL of 1,2-dichloroethane was stirred for 2 h. Separately, 106 mg (0.42 mmol, 1.05 eq) of 3-phenyl-5-(S)-pyrrolidin-2-yl-[1,2,4]oxadiazole hydrochloride (step 4) and (0.46 mmole, 1.10 eq) of N,N-di-isopropylethylamine are stirred in 4.00 mL of 1,2-dichloroethane. After 15 min. the solution of the amine is added to the mixture of the activated acid via a syringe and stirring is continued overnight at room temperature. Then the reaction mixture is heated to reflux for two days. The solvent is evaporated and the residue is taken up in a 1:1 mixture of dichloromethane and water. The aqueous layer is extracted with dichloromethane and the combined organic layers are washed sequentially with 5% citric acid solution, 10% sodium carbonate solution, water and brine, dried with magnesium sulphate, filtered and evaporated under reduced pressure. Purification by preparative thin layer-chromatography on silica gel affords the title compound.

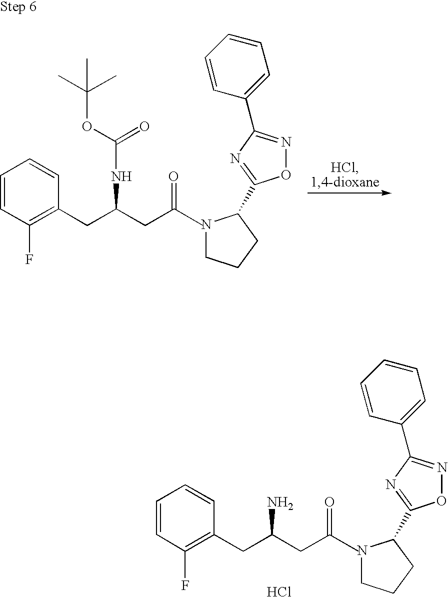

### (R)-3-Amino-4-(2-fluoro-phenyl)-1-[(S)-2-(3-phenyl-[1,2,4]oxadiazol-5-yl)-pyrrolidin-1-yl]-butan-1-one hydrochloride

The intermediate from step 5 is dissolved at 0° C. in 15 mL of 1,4-dioxane that has previously been saturated with hydrochloric acid gas. The reaction mixture is allowed to warm to room temperature over the course of the reaction and stirring is continued until complete conversion is observed by TLC analysis. Evaporation of the solvents affords a solid residue, which is taken up in a mixture of diethyl ether and hexane. The precipitated final compound is filtered off, washed with hexane and dried under reduced pressure.

¹H-NMR (300 MHz, CDCl₃) δ=2.0-3.1 (m, 8H, 4×CH₂), 3.40-4.00 (m, 3H, CH and CH₂), 5.42 (m, 1H, CH), 7.1 (m, 2H, aryl-H), 7.24 (m, 1H, aryl-H), 7.4 (m, 1H, aryl-H), 7.45 (m, 3H, aryl-H), 8.0 (m, 2H, aryl-H), 8.65 (m, 3H, NH).

Using this general procedure the following oxadiazole derivatives were prepared:

### Example 2

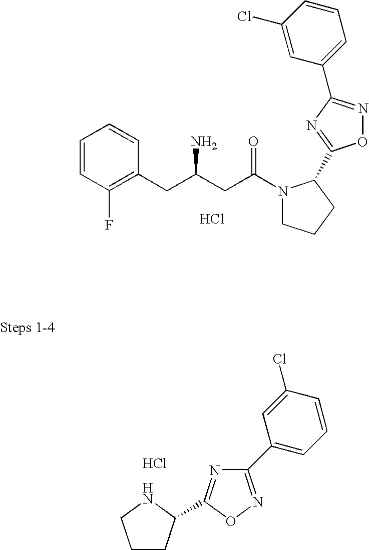

### 3-(3-Chloro-phenyl)-5-(S)-pyrrolidin-2-yl-[1,2,4]oxadiazole hydrochloride

The title compound was prepared according to steps 1-4 of example 1.

¹H-NMR (300 MHz, CDCl₃) δ=2.21 (m, 2H, CH₂), 2.4 (m, 1H, CH), 2.7 (m, 1H, CH), 3.6-3.8 (m, 2H, CH), 5.2 (m, 1H, CH), 7.41 (dd, 1H, aryl-H), 7.51 (ddd, 1H, aryl-H), 7.94 (ddd, 1H, aryl-H), 8.04 (dd, 1H, aryl-H), 10.2-11.2 (s, 2H, NH).

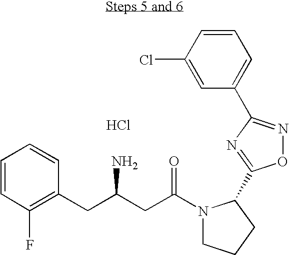

### (R)-3-Amino-1-{(S)-2-[3-(3-chloro-phenyl)[1,2,4]oxadiazol-5-yl)-pyrrolidin-1-yl}-4-(2-fluoro-phenyl)-butan-1-one hydrochloride

The title compound was prepared according to steps 5 and 6 of example 1.

¹H-NMR (300 MHz, CDCl₃) δ=2.2-3.21 (m, 4H, 2×CH₂), 3.5 (m, 2H, CH₂), 3.92 (m, 1H, CH), 5.1 (m, 1H, CH), 7.0-7.5 (m, 6H, aryl-H), 7.9 (m, 1H, aryl-H), 8.0 (dd, 1H, aryl-H), 8.7 (m, 3H, NH₃).

### Example 3

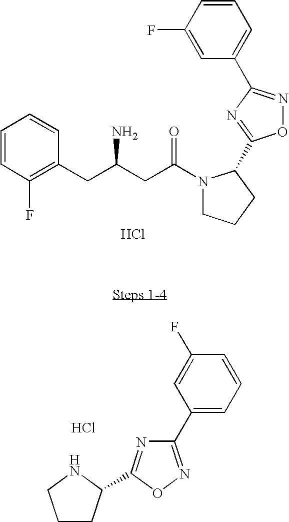

### 3-(3-Fluoro-phenyl)-5-(S)-pyrrolidin-2-yl-[1,2,4]oxadiazole hydrochloride

The title compound was prepared according to steps 1-4 of example 1.

¹H-NMR (300 MHz, CDCl₃) δ=2.17-2.32 (m, 2H, CH₂), 2.45-2.60 (m, 2H, CH₂), 3.64-3.75 (m, 2H, CH₂), 5.2 (m, 1H, CH), 7.20 (m, 1H, aryl-H), 7.43 (m, 1H, aryl-H), 7.76 (m, 1H, aryl-H), 7.86 (m, 1H, aryl-H), 10.4-11.2 (s, 2H, NH₂).

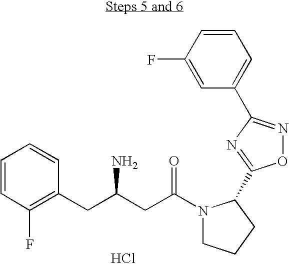

### (R)-3-Amino-1-{(S)-2-[3-(3-fluoro-phenyl) [1,2,4]oxadiazol-5-yl)-pyrrolidin-1-yl}-4-(2-fluoro-phenyl)-butan-1-one hydrochloride

The title compound was prepared according to steps 5 and 6 of example 1.

¹H-NMR (300 MHz, CDCl₃) δ=2.0-3.58 (m, 8H, 4×CH₂), 3.68 (m, 1H, CH), 3.75 (m, 2H, CH₂), 5.3 (m, 1H, CH), 7.10-7.39 (m, 4H, aryl-H), 7.46 (m, 1H, aryl-H), 7.6 (m, 1H, aryl-H), 7.70 (m, 1H, aryl-H), 7.85 (m, 1H, aryl-H), 8.15 (m, 3H, NH₃).

### Example 4

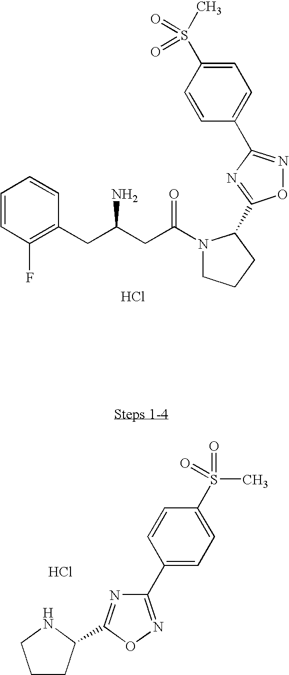

### 3-(4-Methanesulphonyl-phenyl)-5-(S)-pyrrolidin-2-yl-[1,2,4]oxadiazole hydrochloride

The title compound was prepared according to steps 1-4 of example 1.

¹H-NMR (300 MHz, CDCl₃+DMSO-d₆) δ=2.10-2.65 (m, 4H, 2×CH₂), 3.23 (s, 3H, CH₃), 3.46 (m, 2H, CH₂), 5.2 (m, 1H, CH), 8.15-8.3 (m, 4H aryl-H), 10.3 (s, 2H, NH).

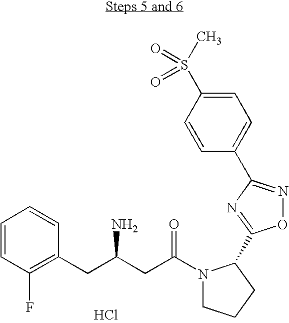

### (R)-3-Amino-4-(2-fluoro-phenyl)-1-{(S)-2-[3-(4-methanesulphonyl-phenyl)[1,2,4]oxadiazol-5-yl]-pyrrolidin-1-yl}-butan-1-one hydrochloride

The title compound was prepared according to steps 5 and 6 of example 1.

¹H-NMR (300 MHz, DMSO-d₆) δ=2.35-1.9 (m, 4H, 2×CH₂), 2.6-3.15 (m, 4H, 2×CH₂), 3.28 (s, 3H, CH₃), 3.6 (m, 1H, CH), 3.7 (m, 2H, CH₂), 5.3 (m, 1H, CH), 7.1-7.4 (m, 4H, aryl-H), 8.08-8.25 (m, 4H, aryl-H), 8.4 (m, 3H, NH₃).

### Example 5

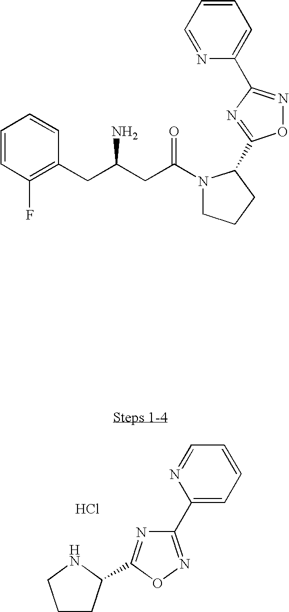

### (S)-2-(5-Pyrrolidin-2-yl-[1,2,4]oxadiazol-3-yl)-pyridine hydrochloride

The title compound was prepared according to steps 1-4 of example 1.

¹H-NMR (300 MHz, CDCl₃+DMSO-d₆) δ=2.21 (m, 2H, CH₂), 2.44 (m, 1H, CH), 2.62 (m, 1H, CH), 3.48 (m, 2H, CH₂), 5.22 (m, 1H, CH), 7.6 (ddd, 1H, py-H), 8.02 (ddd, 1H, py-H), 8.16 (ddd, 1H, py-H), 8.78 (dd, 1H, py-H), 9.8-11.0 (s, 2H, NH₂).

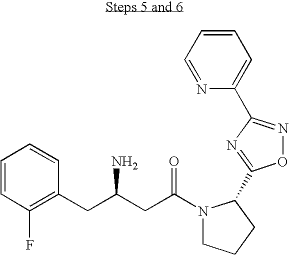

### (R)-3-Amino-4-(2-fluoro-phenyl)-1-[(S)-2-(3-pyridin-2-yl-[1,2,4]oxadiazol-5-yl)-pyrrolidin-1-yl]-butan-1-one

The title compound was prepared according to steps 5 and 6 of example 1.

¹H-NMR (300 MHz, CDCl₃) δ=1.68 (m, 2H, NH₂), 2.38-2.57 (m, 4H, 2×CH₂), 2.77-2.83 (m, 4H, 2×CH₂), 3.48 (m, 2H, CH₂), 3.77 (m, 1H, CH), 5.21 (m, 1H, CH), 7.03-7.28 (m, 4H, aryl-H), 7.42 (m, 1H, py-H), 7.82 (m, 1H, py-H), 8.12 (dd, 1H, py-H), 8.78 (dd, 1H, py-H).

### Example 6

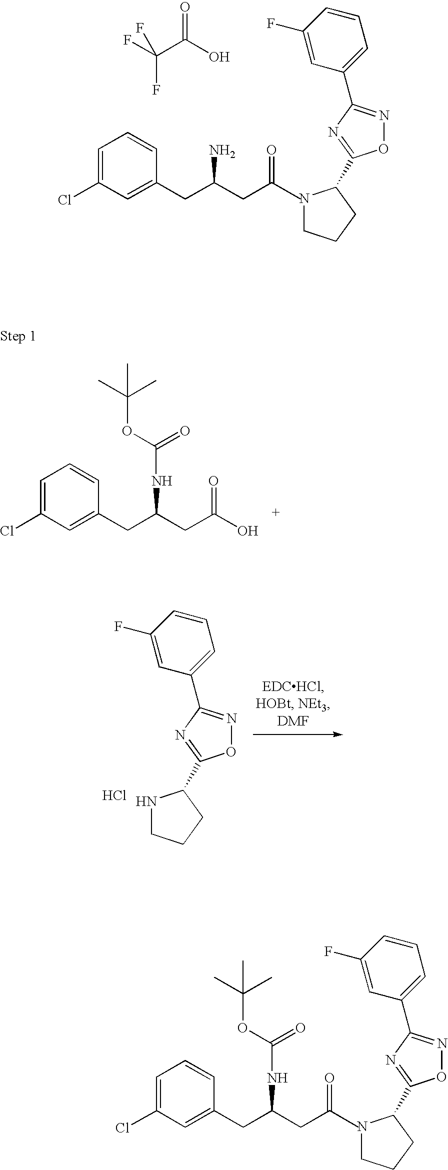

### (R)-(1-(3-Chloro-benzyl)-3-{(S)-2-[3-(3-fluoro-phenyl)-[1,2,4]oxadiazol-5-yl]-pyrrolidin-1-yl}-3-oxo-propyl)-carbamic acid tert-butyl ester

25.0 mg (79.7 mmol, 1.00 eq) of (R)-3-tert-butoxycarbonylamino-4-(3-chloro-phenyl)-butyric acid and 21.5 mg (79.7 mmol, 1.00 eq) of 3-(3-fluoro-phenyl)-5-(S)-pyrrolidin-2-yl-[1,2,4]oxadiazole hydrochloride (prepared according to example 3, steps 1-4) are dissolved in 2.00 mL of N,N-dimethylformamide. Then 16.1 mg (120 mmol, 1.50 eq) 1-hydroxybenzotriazole, 55.0 μL (39.9 mg, 394 mmol, 3.30 eq) of triethylamine and 22.9 mg (120 mmol, 1.50 eq) of 1-ethyl-3-(3-dimethylaminopropyl)carbodiimide hydrochloride are added to the reaction mixture at room temperature. After the resulting solution is stirred for 12 h at room temperature the mixture is poured into brine and is diluted with water. The aqueous mixture is extracted 3 times with ethyl acetate and the combined organic layers are dried with sodium sulphate. The solvent is removed under reduced pressure and the title compound is purified by flash chromatography (silica gel, eluent: cyclo-hexane:ethyl acetate=1:1).

LC/MS (I): rt 3.60; m/z 529 [M+H]⁺, 470 [M-tBu]⁺, 429 [M-Boc]⁺.

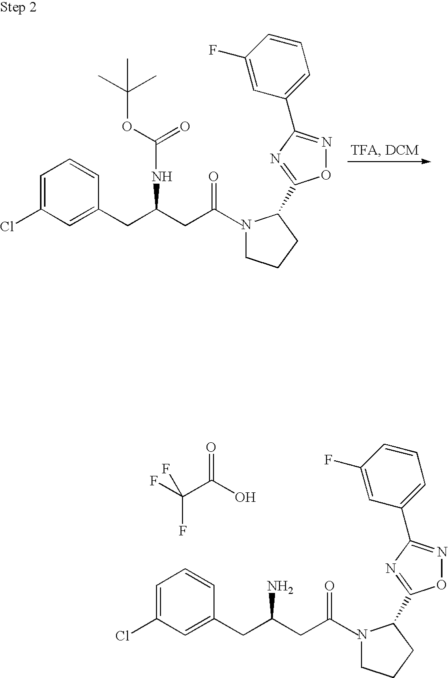

### (R)-3-Amino-4-(3-chloro-phenyl)-1-{(S)-2-[3-(3-fluoro-phenyl)-[1,2,4]oxadiazol-5-yl]-pyrrolidin-1-yl}-butan-1-one trifluoroacetic acid salt

The compound from step 1 is dissolved in 1.00 mL of dichloromethane and is cooled to 0° C. Then 0.50 mL of trifluoroacetic acid is added and the reaction mixture is allowed to warm to room temperature. After 1 h the solvents are removed and the crude product is purified by preparative HPLC to yield the title compound.

LC/MS (I): rt 2.46; m/z 429 [M+H]⁺, 492 [M+Na⁺ MeCN]⁺.

¹H-NMR (300 MHz, DMSO-d₆) δ=2.10 (m, 2H, CH₂), 2.35-2.38 (m, 1H, CH), 2.65 (m, 2H, CH₂), 2.85-2.96 (m, 2H, CH₂), 3.54 (m, 1H, CH), 3.69 (m, 2H, CH₂), 5.27 (m, 1H, CH), 7.1-7.5 (m, 5H, aryl-H), 7.55-7.89 (m, 3H, aryl-H), 7.91 (s, 3H, NH₃).

### Example 7

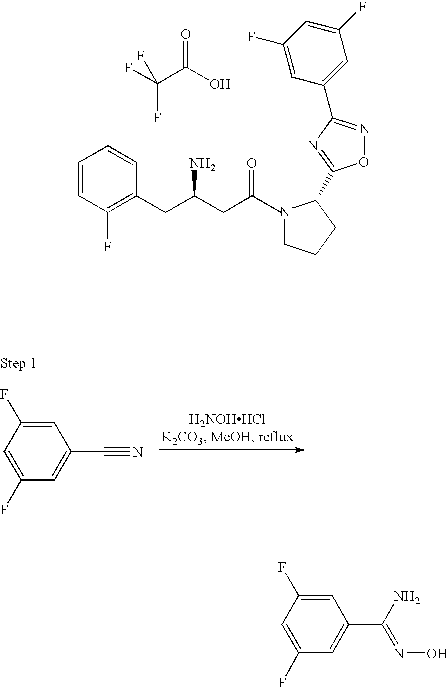

### 3,5-Difluoro-N-hydroxy-benzamidine

100 mg (0.72 mmol, 1.00 eq) of 3,5-difluorobenzonitrile is dissolved in 4.00 mL of methanol and 149 mg (1.08 mmol, 1.50 eq) of potassium carbonate followed by 99.9 mg (1.44 mmol, 2.00 eq) of hydroxylamine hydrochloride are added. The reaction mixture is refluxed for 12 h, filtered and the solvent is evaporated. The crude product is purified by flash chromatography (silica gel, eluent: cyclo-hexane:ethyl acetate=3:2) to yield the title compound.

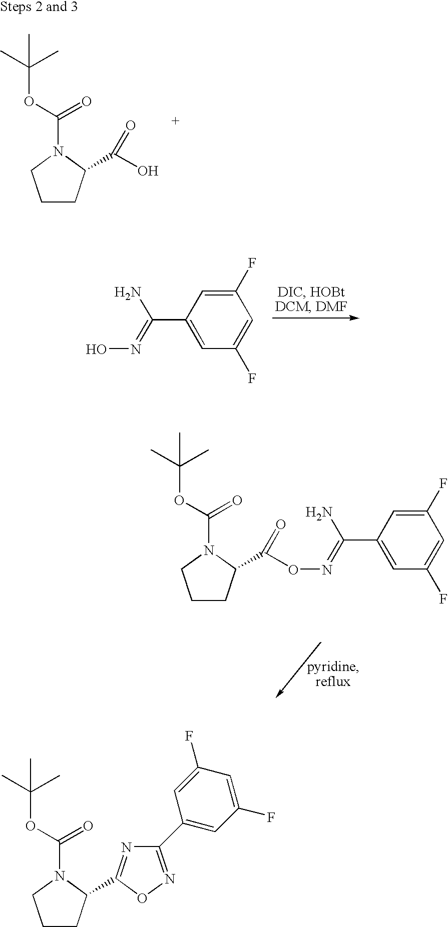

### (S)-2-[3-(3,5-Difluoro-phenyl)-[1,2,4]oxadiazol-5-yl]-pyrrolidine-1-carboxylic acid tert-butyl ester

109 mg (0.63 mmol, 1.00 eq) of 3,5-difluoro-N-hydroxy-benzamidine (Step 1) and 150 mg (0.70 mmol, 1.10 eq) of (S)-pyrrolidine-1,2-dicarboxylic acid 1-tert-butyl ester are dissolved in a mixture of 4 mL of dichloromethane and 0.5 mL of N,N-dimethylformamide. Then 98.9 mg (0.73 mmol, 1.20 eq) 1-hydroxybenzotriazole and 115 μL (92.4 mg, 0.73 mmol, 1.20 eq) N,N′-diisopropylcarbodimide are added to the reaction mixture. After 12 h stirring at room temperature the reaction mixture is poured into brine, and is further diluted with water. The aqueous mixture is extracted 3 times with ethyl acetate and the combined organic layers are washed with 5% citric acid solution, saturated sodium bicarbonate solution, brine and dried with sodium sulphate. The solvent is evaporated and the residue is purified by flash chromatography (silica gel, eluent: cyclo-hexane:ethyl acetate=1:1). 203 mg (0.55 mmol) of the O-acyl amidoxime is dissolved in 4.00 mL of pyridine and the solution is heated to reflux for 12 h. Then the pyridine is removed under reduced pressure and the residue is purified via flash chromatography (silica gel, eluent: cyclo-hexane:ethyl acetate=4:1) to yield the title compound.

LC/MS (I): rt 3.34, λₘₐₓ=234 nm (s), 277 nm (w).

¹H-NMR (300 MHz, DMSO-d₆) δ=1.19-1.39 (d, 9H, 3×CH₃), 1.94-2.04 (m, 3H, CH and CH₂), 2.38-2.40 (m, 1H, CH), 3.23-3.56 (m, 2H, CH₂), 5.10 (m, 1H, CH), 7.48 (m, 1H, aryl-H), 7.59 (m, 2H, aryl-H).

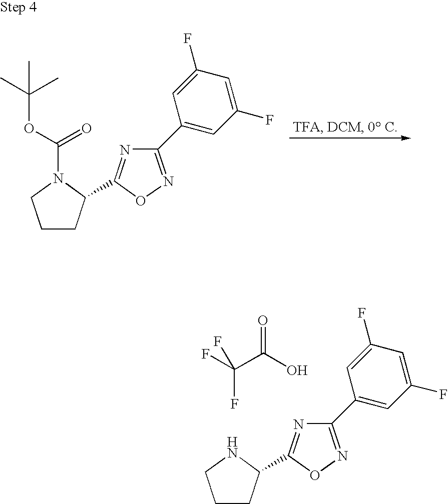

### 3-(3,5-Difluoro-phenyl)-5-(S)-pyrrolidin-2-yl-[1,2,4]oxadiazole trifluoroacetic acetate

46.0 mg (131 μmol, 1.00 eq) of the compound from step 3 is dissolved in 1.00 mL of dichloromethane. The solution is cooled to 0° C. and 0.50 mL of trifluoroacedic acid is added. The reaction mixture is allowed to warm to room temperature and after 1 h the solvents are evaporated to yield the crude title compound, which is dried under reduced pressure and used without further purification in step 5. LC/MS (I): rt 1.85; m/z 252 [M+H]⁺, 293 [M+H+MeCN]⁺.

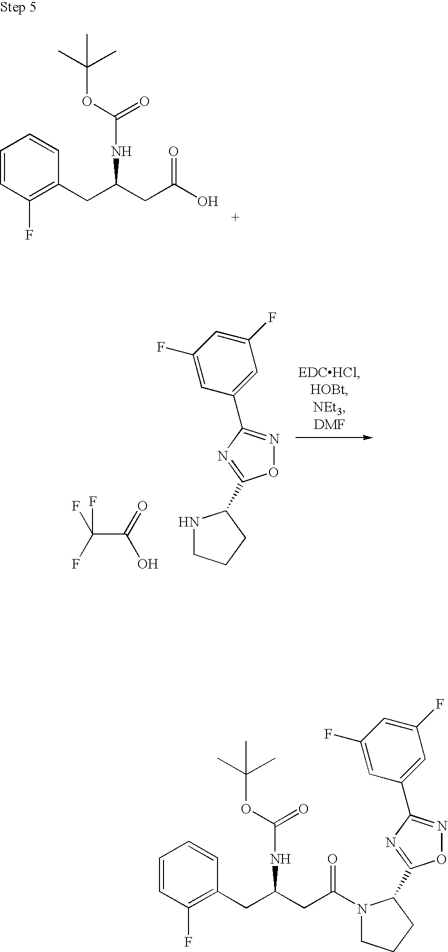

### (R)-[3-{(S)-2-[3-(3,5-Difluoro-phenyl)-[1,2,4]oxadiazol-5-yl]-pyrrolidin-1-yl}-1-(2-fluoro-benzyl)-3-oxo-propyl]-carbamic acid tert-butyl ester

14.9 mg (110 mmol, 1.50 eq) of 1-hydroxybenzotriazole, 36.9 μL (26.8 mg, 264 μmol, 3.60 eq) triethylamine and 21.1 mg (110 μmol, 1.50 eq) of 1-ethyl-3-(3-dimethylaminopropyl)carbodiimide hydrochloride are added to a stirred solution of 21.8 mg (73.4 mmol, 1.00 eq) of (R)-3-tert-butoxycarbonylamino-4-(2-fluoro-phenyl)-butyric acid in 1.00 mL of N,N-dimethylformamide. The reaction mixture is stirred for 15 min., after which a solution of 26.8 mg (73.4 mmol, 1.00 eq) of the crude deprotected pyrrolidine compound from step 4 in 1.00 mL of N,N-dimethylformamide is added. The resulting solution is stirred for 12 h, then poured into brine and diluted with water. The aqueous mixture is extracted 3 times with ethyl acetate and the combined organic layers are washed with 5% citric acid solution, saturated sodium bicarbonate solution, brine and are dried with sodium sulphate. The solvent is evaporated and the residue is subjected to flash chromatography (silica gel, eluent: cyclo-hexane:ethyl acetate=3:2) to yield the title compound.

LC/MS (I): rt 3.48; m/z 431 [M+H-Boc)⁺.

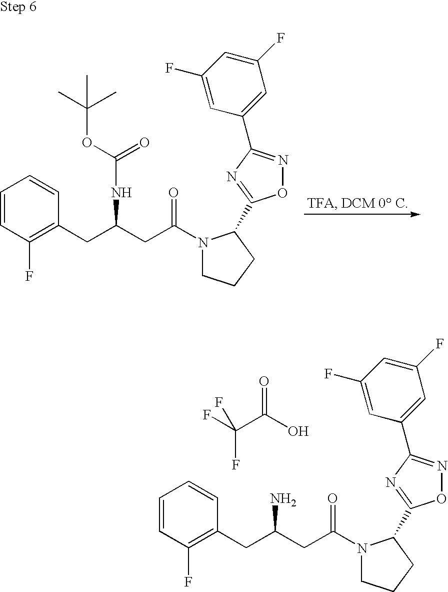

### (R)-3-Amino-1-{(S)-2-[3-(3,5-difluoro-phenyl)-[1,2,4]oxadiazol-5-yl)-pyrrolidin-1-yl}-4-(2-fluoro-phenyl)-butan-1-one trifluoracetic acid salt

42.0 mg (79.2 μmol, 1.00 eq) of the compound from step 5 is dissolved in 1.00 mL of dichloromethane. The solution is cooled to 0° C. and 0.50 mL of trifluoroacetic acid is added. The reaction mixture is allowed to warm to room temperature and after 30 min the solvent is evaporated to yield the crude title compound, which is purified by preparative HPLC.

LC/MS (I): rt 2.58; m/z 453 [M+Na]⁺, 472 [M+H+MeCN]⁺.

¹H-NMR (300 MHz, DMSO-d₆) δ=2.08 (m, 3H, CH and CH₂), 2.35 (m, 1H, CH), 2.64 (m, 2H, CH₂), 2.97 (m, 2H, CH₂), 3.50 (m, 1H, CH), 3.60-3.75 (m, 2H, CH₂), 5.26 (m, 1H, CH), 7.10-7.17 (m, 2H, aryl-H), 7.2-7.35 (m, 2H, aryl-H), 7.4-7.6 (m, 3H, aryl-H), 7.8-8.1 (s, 3H, NH₃).

### Example 8

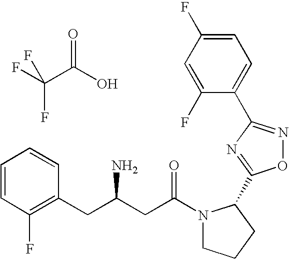

### (R)-3-Amino-1-{(S)-2-[3-(2,4-difluoro-phenyl)-[1,2,4]oxadiazol-5-yl)-pyrrolidin-1-yl}-4-(2-fluoro-phenyl)-butan-1-one trifluoracetic acid salt

The title compound can be prepared according to steps 1-6 for example 7 by using 2,4-difluoro-benzonitrile as starting material.

LC/MS (I): rt 2.49; m/z 453 [M+Na]⁺, 472 [M+H+MeCN]⁺.

¹H-NMR (300 MHz, DMSO-d₆) δ=1.96 (m, 3H, CH and CH₂), 2.35 (m, 1H, CH), 2.65 (m, 2H, CH₂), 2.97 (m, 2H, CH₂), 3.51 (m, 1H, CH), 3.67 (m, 2H, CH₂), 5.27 (m, 1H, CH), 7.09-7.23 (m, 2H, aryl-H), 7.27-7.32 (m, 3H, aryl-H), 7.45 (m, 1H, aryl-H), 7.89-8.03 (m, 4H, NH₃, aryl-H)

### Example 9

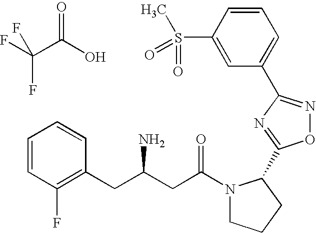

### (R)-3-Amino-1-{(S)-2-[3-(3-methanesulphonyl-phenyl)-[1,2,4]oxadiazol-5-yl)-pyrrolidin-1-yl}-4-(2-fluoro-phenyl)-butan-1-one trifluoracetic acid salt

The title compound can be prepared according to steps 1-6 for example 7 by using 3-methanesulfonyl-benzonitrile as starting material.

LC/MS (I): rt 2.31; m/z 473 [M+H]⁺, 495 [M+Na]⁺.

¹H-NMR (300 MHz, DMSO-d₆) δ=2.09 (m, 3H, CH and CH₂), 2.35 (m, 1H, CH), 2.66 (m, 2H, CH₂), 2.96 (m, 2H, CH₂), 3.26 (s, 3H, CH₃), 3.52 (m, 1H, CH), 3.67 (m, 2H, CH₂), 5.27 (m, 1H, CH), 7.14 (m, 2H, aryl-H), 7.30 (m, 2H, aryl-H), 7.81 (dd, 1H, aryl-H), 7.9-8.0 (s, 3H, NH₃), 8.11 (d, 1H, aryl-H), 8.24 (d, 1H, aryl-H), 8.37 (s, 1H, aryl-H).

### Example 10

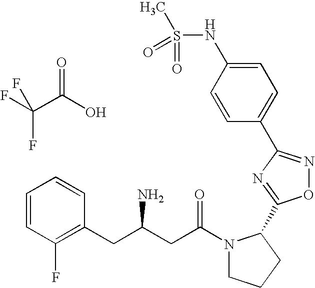

### N-[4-(5-{(S)-1-[(R)-3-Amino-4-(2-fluoro-phenyl)-butyryl]-pyrrolidin-2-yl}-[1,2,4]oxadiazol-3-yl)-phenyl]-methanesulphonamide trifluoroacetic acid salt

The title compound can be prepared according to steps 1-6 for example 7 by using N-(4-cyano-phenyl)-methanesulfonamide as starting material.

LC/MS (I): rt 2.15; m/z 488 [M+H]⁺, 529 [M+MeCN]⁺.

¹H-NMR (300 MHz, DMSO-d₆) δ=2.06 (m, 2H, CH and CH₂), 2.34 (m, 1H, CH), 2.64 (m, 2H, CH₂), 2.94-2.99 (m, 2H, CH₂), 3.06 (s, 3H, CH₃), 3.48-3.64 (m, 3H, CH and CH₂), 5.22 (m, 1H, CH), 7.09-7.16 (m, 2H, aryl-H), 7.28-7.34 (m, 4H, aryl-H), 7.84-7.94 (m, 4H, NH₂ and 2× aryl-H), 10.1 (s, 1H, NH).

### Example 11

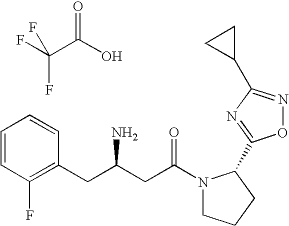

### (R)-3-Amino-1-[(S)-2-(3-cyclopropyl-[1,2,4]oxadiazol-5-yl)-pyrrolidin-1-yl]-4-(2-fluorophenyl)-butan-1-one trifluoracetic acid salt

The title compound was prepared according to steps 1-6 for example 7 by using cyclopropanecarbonitrile as starting material.

LC/MS (I): rt 2.12; m/z 359 [M+H]⁺, 400 [M+MeCN]⁺.

¹H-NMR (300 MHz, DMSO-d₆) δ=0.82 (m, 2H, c-propyl-CH₂), 1.02 (m, 2H, c-propyl-CH₂), 1.95-2.04 (m, 4H, 2×CH and CH₂), 2.27 (m, 1H, CH), 2.62 (m, 2H, CH₂), 2.94 (m, 2H, CH₂), 3.45 (m, 1H, CH), 3.66 (m, 2H, CH₂), 5.10 (m, 1H, CH), 7.15 (m, 2H, aryl-H), 7.31 (m, 2H, aryl-H), 7.94 (s, 3H, NH₃).

### Example 12

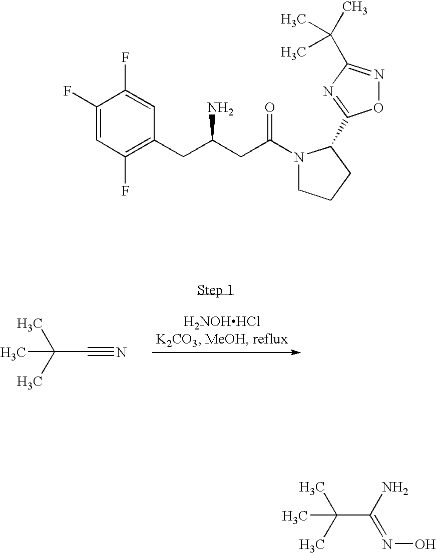

### N-Hydroxy-2,2-dimethyl-propionamidine

26.6 mL (0.24 mol, 1.00 eq) of trimethylacetonitrile is dissolved in 500 mL of methanol and 50.0 g (0.36 mol, 1.50 eq) of potassium carbonate followed by 33.0 g (0.48 mol, 2.00 eq) of hydroxylamine hydrochloride are added. The reaction mixture is refluxed for 4 h, filtered and the solvent is evaporated. The crude product is dissolved in methanol and separated from the inorganic salts by filtration. The filtrate is again evaporated and the crude product (12.5 g, 45%) is used in the next step without further purification.

LC/MS (I): rt 0.68; m/z 117 [M+H]⁺, 158 [M+H+MeCN]⁺.

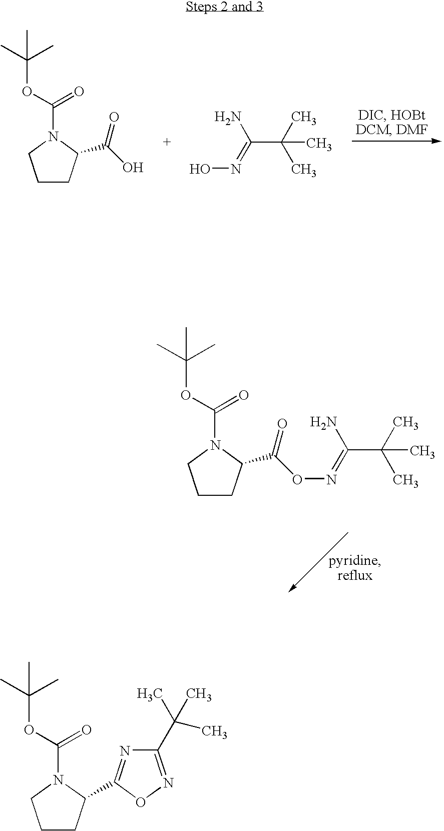

### (S)-2-(3-tert-Butyl-[1,2,4]oxadiazol-5-yl)-pyrrolidine-1-carboxylic acid tert-butyl ester

3.00 g (25.8 mmol, 1.00 eq) of N-hydroxy-2,2-dimethyl-propionamidine (Step 1) and 5.55 g (25.8 mol, 1.10 eq) of (S)-pyrrolidine-1,2-dicarboxylic acid 1-tert-butyl ester are dissolved in a mixture of 50 mL of dichloromethane. Then 3.9 mg (31.0 mmol, 1.20 eq) N,N′-diisopropylcarbodimide are added to the reaction mixture. After 6 h stirring at room temperature the reaction mixture is evaporated. The crude O-acyl amidoxime is dissolved in 100 mL of pyridine and the solution is heated to reflux overnight (12 h). Then the pyridine is removed under reduced pressure and the residue is purified via flash chromatography (silica gel, eluent: cyclo-hexane:ethyl acetate=4:1) to yield the title compound.

LC/MS (I): rt 4.38; m/z 237, 241, 281, 296.

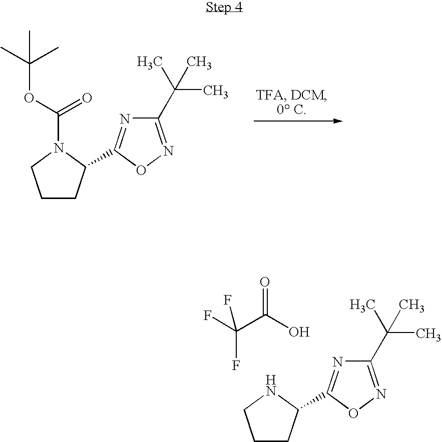

### 3-tert-Butyl-5-(S)-pyrrolidin-2-yl-[1,2,4]oxadiazole trifluoro-acetic acid salt

4.08 g (13.8 mmol, 1.00 eq) of (S)-2-(3-tert-butyl-[1,2,4]oxadiazol-5-yl)-pyrrolidine-1-carboxylic acid tert-butyl ester (Step 3) is dissolved in 20.0 mL of dichloromethane. The solution is cooled to 0° C. and 6.00 mL of trifluoroacetic acid is added. The reaction mixture is allowed to warm to room temperature and after 2 h the solvents are evaporated to yield the crude title compound, which is dried under reduced pressure and used without further purification in step 5.

LC/MS (I): rt 1.68; m/z 196 [M+H]⁺, 237 [M+H+MeCN]⁺. 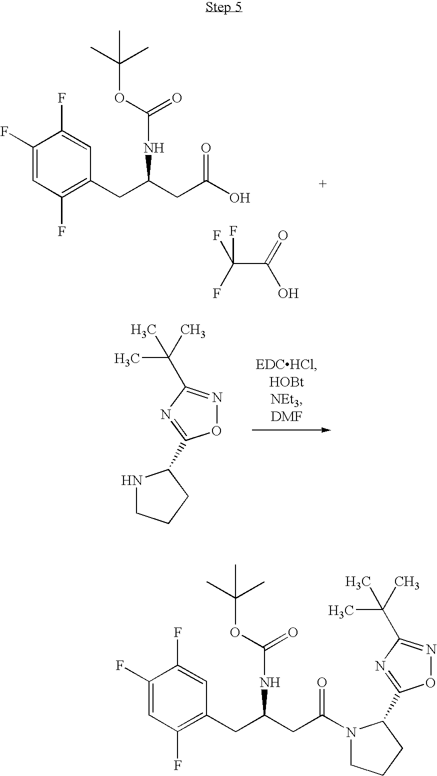
[(R)-3-[(S)-2-(3-tert-Butyl-[1,2,4]oxadiazol-5-yl)-pyrrolidin-1-yl]-3-oxo-1-(2,4,5-trifluorobenzyl)-propyl]-carbamic acid tert-butyl ester

655 mg (4.85 mmol, 1.50 eq) of 1-hydroxybenzotriazole, 1.16 mL (8.41 mmol, 2.60 eq) triethylamine and 930 mg (4.85 mmol, 1.50 eq) of 1-ethyl-3-(3-dimethylaminopropyl)carbodiimide hydrochloride are added to a stirred solution of 1.08 g (3.23 mmol, 1.00 eq) of (R)-3-tert-butoxycarbonylamino-4-(2,4,5-trifluoro-phenyl)-butyric acid in 30 mL of N,N-dimethylformamide. The reaction mixture is stirred for 10 min, after which a solution of 1.00 g (3.23 mmol, 1.00 eq) of the trifluoroacitic acid salt of 3-tert-butyl-5-(S)-pyrrolidin-2-yl-[1,2,4]oxadiazole from step 4 in 20 mL of N,N-dimethylformamide is added at 0° C. The resulting solution is stirred for 12 h, then poured into brine and diluted with water. The aqueous mixture is extracted 3 times with ethyl acetate and the combined organic layers are washed with 5% citric acid solution, saturated sodium bicarbonate solution, brine and are dried with sodium sulphate. The solvent is evaporated and the residue is subjected to flash chromatography (silica gel, eluent: cyclo-hexane:ethyl acetate=70:30) to yield 737 mg (45%) of the pure title compound.

LC/MS (I): rt 4.76; m/z 411 [M+H-Boc]⁺, 511 [M+H]⁺, 533 [M+Na]⁺, 574 [M+ACN+Na]⁺.

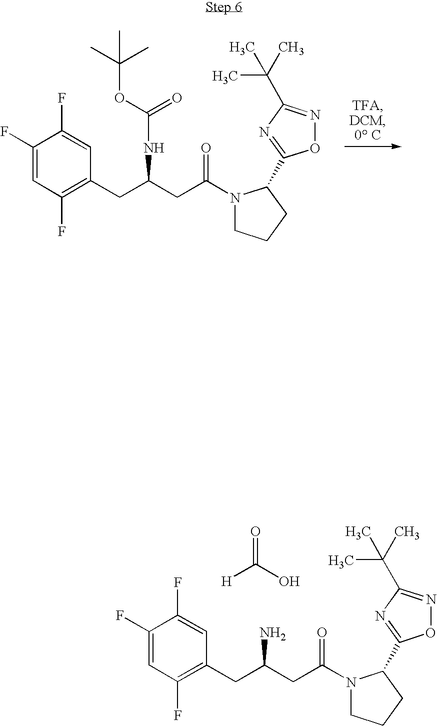

### (R)-3-Amino-1-[(S)-2-(3-tert-butyl-[1,2,4]oxadiazol-5-yl)-pyrrolidin-1-yl]-4-(2,4,5-trifluorophenyl)-butan-1-one formate salt

737 mg (1.44 mmol, 1.00 eq) of [(R)-3-[(S)-2-(3-tert-butyl-[1,2,4]oxadiazol-5-yl)pyrrolidin-1-yl]-3-oxo-1-(2,4,5-trifluoro-benzyl)-propyl]-carbamic acid tert-butyl ester from step 5 is dissolved in 3.00 mL of dichloromethane. The solution is cooled to 0° C. and 0.90 mL of trifluoroacetic acid is added. The reaction mixture is allowed to warm to room temperature and after 1 h the solvent is evaporated to yield the crude title compound, which is purified by preparative LC/MS running with formic acid as co-eluent.

LC/MS (I): rt 4.38; m/z 411 [M+H]⁺, 452 [M+H+MeCN]⁺.

¹H-NMR (400 MHz, DMSO-d₆) δ=1.26 (s, 9H, CH₃), 1.95-2.04 (m, 3H, CH, CH₂), 2.28-2.33 (m, 1H, CH), 2.63-2.78 (m, 2H, CH₂), 2.89 (d, 2H, CH₂), 3.45-3.52 (m, 2H, CH₂), 3.65-3.68 (m, 2H, CH₂), 5.16-5.46 (m, 1H, CH), 7.47-7.59 (m, 2H, aryl-H).

### Example 13

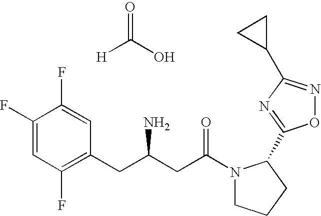

### (R)-3-Amino-1-[(S)-2-(3-cyclopropyl-[1,2,4]oxadiazol-5-yl)-pyrrolidin-1-yl]-4-(2,4,5-trifluoro-phenyl)-butan-1-one formate salt

The title compound can be prepared according to the procedure described for example 12 for steps 1-6 by using cyclopropanecarbonitrile in step 1.

LC/MS (III): rt 6.38; m/z 395 [M+H]⁺, 436 [M+H+MeCN]⁺.

¹H-NMR (400 MHz, DMSO-d₆) δ=0.62-0.89 (m, 2H, CH₂), 0.96-1.09 (m, 2H, CH₂), 1.85-2.07 (m, 5H, CH, 2×CH₂), 2.23-2.30 (m, 1H, CH), 2.72-2.76 (m, 3H, CH, CH₂), 3.40-3.64 (m, 3H, CH, CH₂), 5.06-5.34 (m, 1H, CH), 7.39-7.42 (m, 2H, aryl-H), 8.16 (s, 1H, HCOOH).

### Example 14

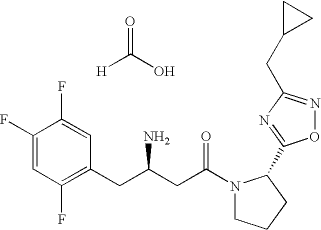

(R)-3-Amino-1-[(S)-2-(3-cyclopropylmethyl-[1,2,4]oxadiazol-5-yl)-pyrrolidin-1-yl]-4-(2,4,5-trifluoro-phenyl)-butan-1-one formate salt

The title compound can be prepared according to the procedure described for example 12 for steps 1-6 by using cyclopropyl-acetonitrile in step 1.

LC/MS (III): rt 7.85; m/z 409 [M+H]⁺, 450 [M+H+MeCN]⁺.

¹H-NMR (400 MHz, DMSO-d₆) δ=0.16-0.17 (m, 2H, CH₂), 0.43-0.45 (m, 2H, CH₂), 0.97-1.00 (m, 1H, CH), 1.91-1.99 (m, 3H, CH, CH₂), 2.28-2.31 (m, 2H, CH₂), 2.56-2.61 (m, 3H, CH, CH₂), 2.81-2.83 (m, 2H, CH₂), 3.49-3.64 (m, 3H, CH, CH₂), 5.14-5.45 (m, 1H, CH), 7.43-7.55 (m, 2H, aryl-H), 8.14 (s, 1H, HCOOH).

### Example 15

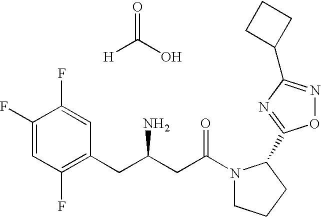

### (R)-3-Amino-1-[(S)-2-(3-cyclobutyl-[1,2,4]oxadiazol-5-yl)-pyrrolidin-1-yl]-4-(2,4,5-trifluoro-phenyl)-butan-1-one formate salt

The title compound can be prepared according to the procedure described for example 12 for steps 1-6 by using cyclobutanecarbonitrile in step 1.

LC/MS (IV): rt 4.79; m/z 409 [M+H]⁺, 450 [M+H+MeCN]⁺.

¹H-NMR (400 MHz, DMSO-d₆) δ=1.85-2.09 (m, 5H, CH, 2×CH₂), 2.21-2.27 (m, 5H, CH, 2×CH₂), 2.74-2.76 (m, 2H, CH₂), 3.32-3.66 (m, 7H, CH, 3×CH₂), 5.15-5.44 (m, 1H, CH), 7.43-7.51 (m, 2H, aryl-H), 8.18 (s, 1H, HCOOH).

### Example 16

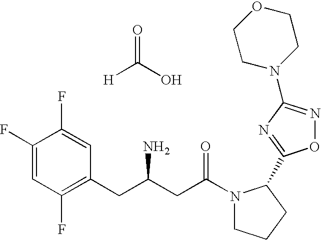

### (R)-3-Amino-1-[(S)-2-(3-morpholin-4-v1-[1,2,4]oxadiazol-5-yl)-pyrrolidin-1-yl]-4-(2,4,5-trifluoro-phenyl)-butan-1-one formate salt

The title compound can be prepared according to the procedure described for example 12 for steps 1-6 by using morpholine-4-carbonitrile in step 1.

LC/MS (III): rt 7.21; m/z 440 [M+H]⁺, 462 [M+Na]⁺, 481 [M+H+MeCN]⁺.

¹H-NMR (400 MHz, DMSO-d₆) δ=1.92-2.02 (m, 3H, CH, CH₂), 2.22-2.25 (m, 2H, CH₂), 2.54-2.58 (m, 2H, CH₂), 2.81-2.83 (m, 2H, CH₂), 3.23-3.63 (m, 11H, CH, 5×CH₂), 5.00-5.31 (m, 1H, CH), 7.43-7.56 (m, 2H, aryl-H), 8.13 (s, 1H, HCOOH).

### Example 17

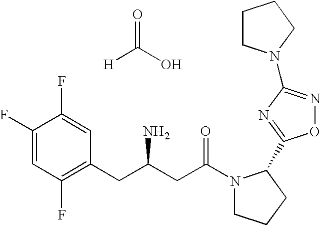

### (R)-3-Amino-1-[(S)-2-(3-pyrrolidin-1-yl-[1,2,4]oxadiazol-5-yl)-pyrrolidin-1-yl]-4-(2,4,5-trifluoro-phenyl)-butan-1-one formate salt

The title compound can be prepared according to the procedure described for example 12 for steps 1-6 by using pyrrolidine-4-carbonitrile in step 1.

LC/MS (III): rt 7.21; m/z 424 [M+H]⁺, 465 [M+H+MeCN]⁺.

¹H-NMR (400 MHz, DMSO-d₆) δ=1.90-2.01 (m, 4H, 2×CH, CH₂), 2.24-2.29 (m, 1H, CH), 2.75-2.80 (m, 3H, CH, CH₂), 3.24-3.65 (m, 11H, CH, 5×CH₂), 5.01-5.31 (m, 1H, CH), 7.42-7.54 (m, 2H, aryl-H), 8.19 (s, 1H, HCOOH).

### Example 18

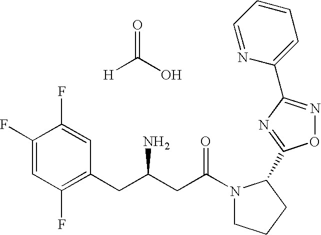

### (R)-3-Amino-1-F(S)-2-(3-pyridin-2-yl-[1,2,4]oxadiazol-5-yl)-pyrrolidin-1-yl-4-(2,4,5-trifluoro-phenyl)-butan-1-one formate salt

The title compound can be prepared according to the procedure described for example 12 for steps 1-6 by using pyridine-2-carbonitrile in step 1.

LC/MS (IV): rt 3.37; m/z 432 [M+H]⁺, 454 [M+Na]⁺.

¹H-NMR (300 MHz, DMSO-d₆) δ=1.89-2.12 (m, 3H, CH, CH₂), 2.15-2.56 (m, 2H, CH₂), 2.75-2.81 (m, 3H, CH, CH₂), 3.44-3.70 (m, 3H, CH, CH₂), 5.24-5.53 (m, 1H, CH), 7.35-7.44 (m, 2H, py-H), 7.52-7.60 (m, 1H, aryl-H), 7.94-8.01 (m, 2H, py-H), 8.12 (s, 1H, HCOOH), 8.67-8.69 (m, 1H, py-H).

### Example 19

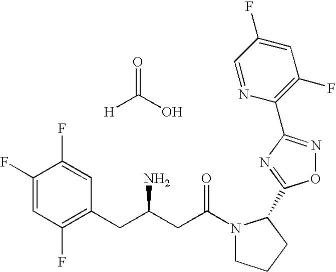

### (R)-3-Amino-1-{(S)-2-[3-(3,5-difluoro-pyridin-2-yl)-[1,2,4]oxadiazol-5-yl]-pyrrolidin-1-yl}-4-(2,4,5-trifluoro-phenyl)-butan-1-one formate salt

The title compound can be prepared according to the procedure described for example 12 for steps 1-6 by using 3,5-difluoro-pyridine-2-carbonitrile in step 1.

LC/MS (IV): rt 4.36; m/z 468 [M+H]⁺, 490 [M+Na]⁺, 509 [M+H+MeCN]⁺.

¹H-NMR (300 MHz, DMSO-d₆) δ=2.05-2.08 (m, 3H, CH, CH₂), 2.31-2.38 (m, 2H, CH₂), 2.47-2.83 (m, 2H, CH₂), 2.81-2.83 (m, 2H, CH₂), 3.41-3.74 (m, 2H, CH₂), 5.27-5.60 (m, 1H, CH), 7.42-7.53 (m, 2H, aryl-H), 8.13 (s, 1H, HCOOH), 8.19-8.24 (m, 1H, py-H), 8.70-8.75 (m, 1H, py-H).

### Example 20

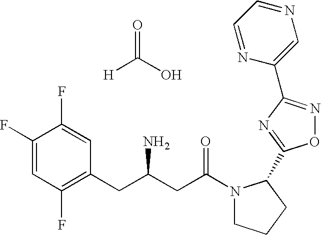

### (R)-3-Amino-1-[(S)-2-(3-pyrazin-2-v1-[1,2,4]oxadiazol-5-yl)-pyrrolidin-1-yl]-4-(2,4,5-trifluoro-phenyl)-butan-1-one formate salt

The title compound can be prepared according to the procedure described for example 12 for steps 1-6 by using pyrazine-2-carbonitrile in step 1.

LC/MS (III): rt 5.82; m/z 433 [M+H]⁺, 455 [M+Na]⁺.

### Example 21

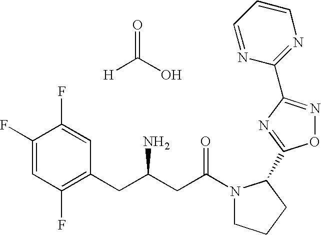

### (R)-3-Amino-1-[(S)-2-(3-pyrimidin-2-yl-[1,2,4]oxadiazol-5-yl)-pyrrolidin-1-yl]-4-(2,4,5-trifluoro-phenyl)-butan-1-one

The title compound can be prepared according to the procedure described for example 12 for steps 1-6 by using pyrimidine-2-carbonitrile in step 1.

LC/MS (III): rt 5.74; m/z 433 [M+H]⁺, 455 [M+Na]⁺.

### Example 22

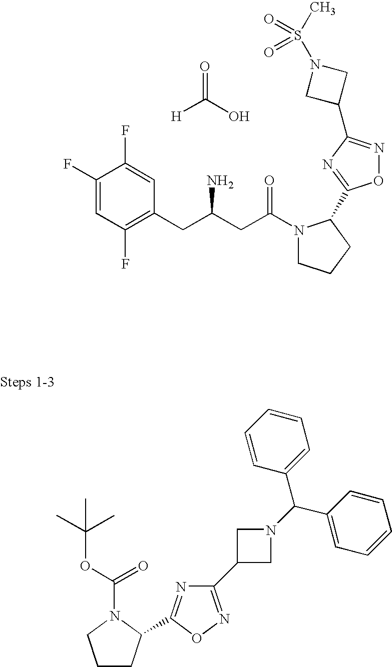

### (S)-2-[3-(1-Benzhydryl-azetidin-3-yl)-[1,2,4]oxadiazol-5-yl]-pyrrolidine-1-carboxylic acid tert-butyl ester

The title compound can be prepared according to steps 1-3 of example 12 by employing 1-benzhydryl-azetidine-3-carbonitrile in step 1.

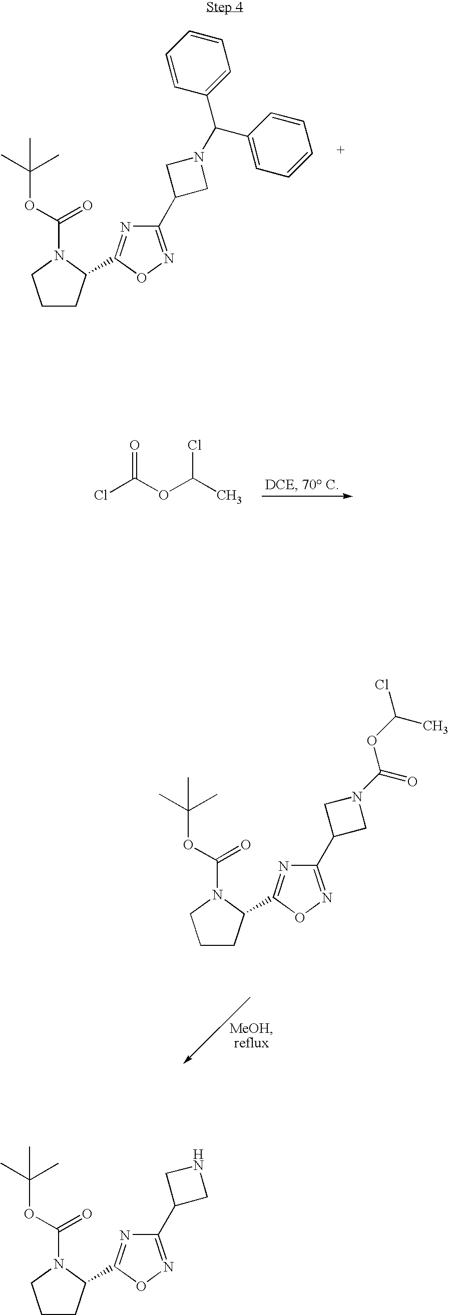

### (S)-2-(3-Azetidin-3-Y1-[1,2,4]oxadiazol-5-yl)-pyrrolidine-1-carboxylic acid tert-butyl ester

264 mg (0.57 mmol, 1.00 eq) of (S)-2-[3-(1-benzhydryl-azetidin-3-yl)-[1,2,4]oxadiazol-5-yl]-pyrrolidine-1-carboxylic acid tert-butyl ester are dissolved in 4 mL of 1,2-dichloroethane and 68.0 μL (0.63 mmol, 1.10 eq) of 1-chloroethylchloroformate is added. The reaction mixture is heated to 70° C. for 1 h, the solvent is evaporated and the intermediate is purified by flash chromatography on silica gel (cyclo-hexane:ethyl acetate 50:50). The carbamate intermediate is then heated to reflux for 1 h in 4 mL of methanol. Afterwards the solvent is evaporated and the crude product is used in the next step without further purification.

LC/MS (I): rt 1.79; m/z 239, 218, 295 [M+H]⁺, 313 [M+Na]⁺, 336 [M+H+MeCN]⁺.

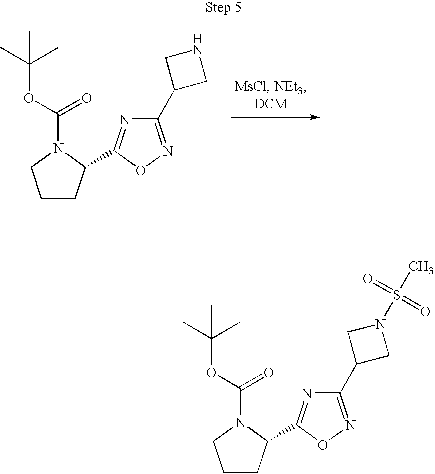

### (S)-2-[3-(1-Methanesulphonyl-azetidin-3-yl)-[1,2,4]oxadiazol-5-yl]-pyrrolidine-1-carboxylic acid tert-butyl ester

20 mg (0.07 mmol, 1.00 eq) of (S)-2-(3-azetidin-3-yl-[1,2,4]oxadiazol-5-yl)-pyrrolidine-1-carboxylic acid tert-butyl ester from step 4 are dissolved in 2 mL of dichloromethane and 31.3 μL (0.22 mmol, 3.30 eq) of triethylamine are added. The solution is cooled to 0° C. and 11.6 μL (0.15 mmol, 2.20 eq) of methanesulphonyl chloride is added and the reaction mixture is stirred for 2 h at 0° C. Then the solution is washed with saturated sodium bicarbonate solution, water and brine, dried over sodium sulphate and evaporated under reduced pressure. The residue is purified by column chromatography (cyclo-hexane:ethyl acetate 50:50) to yield the title compound.

LC/MS (I): 15-95 gradient rt 2.04; m/z 273 [M-Boc]⁺, 314, 395, 436.

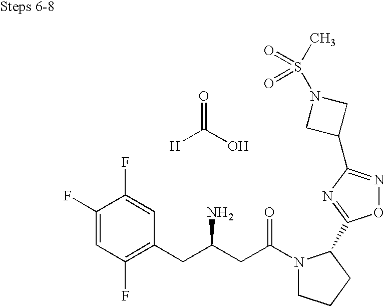

### (R)-3-Amino-1-{(S)-2-[3-(1-methanesulphonyl-azetidin-3-yl)-[1,2,4]oxadiazol-5-yl]-pyrrolidin-1-yl}-4-(2,4,5-trifluoro-phenyl)-butan-1-one formate salt

The title compound can be prepared by employing the deprotection, peptide coupling and deprotection sequence described for example 12, steps 4-6 with (S)-2-[3-(1-methanesulphonyl-azetidin-3-yl)-[1,2,4]oxadiazol-5-yl]-pyrrolidine-1-carboxylic acid tert-butyl ester from step 5.

LC/MS (III): rt 6.39, m/z 488 [M+H]⁺, 510 [M+Na]⁺.

¹H-NMR (300 MHz, DMSO-d₆) δ=1.95-2.06 (m, 3H, CH, CH₂), 2.20-2.34 (m, 1H, CH), 2.52-2.59 (m, 2H, CH₂), 2.80-2.85 (m, 2H, CH₂), 3.02 (d, 3H, CH₃), 3.45-3.69 (m, 3H, CH, CH₂), 3.95-4.03 (m, 3H, CH, CH₂), 4.18-4.25 (m, 2H, CH₂), 5.17-5.45 (m, 1H, CH), 7.41-7.47 (m, 2H, aryl-H), 8.13 (s, 1H, HCOOH).

### Example 23

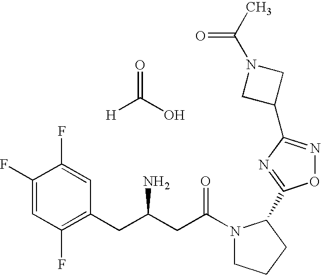

Steps 1-4 can be performed according to example 22, steps 1-4.

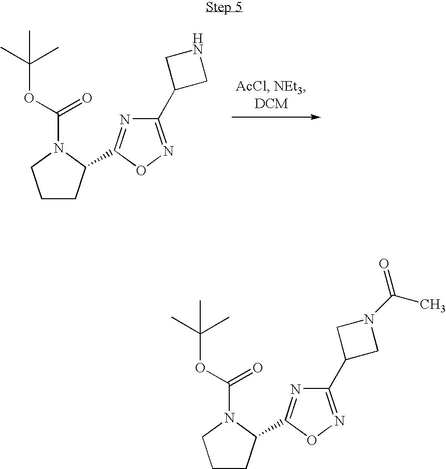

### (S)-2-[3-(1-Acetyl-azetidin-3-yl)-[1,2,4]oxadiazol-5-yl]-pyrrolidine-1-carboxylic acid tert-butyl ester

73 mg (0.25 mmol, 1.00 eq) of (S)-2-(3-azetidin-3-yl-[1,2,4]oxadiazol-5-yl)-pyrrolidine-1-carboxylic acid tert-butyl ester (step 4, example 22) are dissolved in 2 mL of dichloroethane. Subsequently, 138 μL (0.99 mmol, 4.00 eq) of triethylamine and 35.3 μL (0.50 mmol, 2.00 eq) of acetylchloride are added, and the mixture is stirred for 2 h at room temperature. Afterwards the solution is washed with saturated sodium bicarbonate solution, 10% aqueous ammonium chloride solution and brine, dried with sodium sulphate and evaporated under reduced pressure. The crude product is purified by flash chromatography on silica gel to yield the title compound.

LC/MS (I): rt 2.25; m/z 281, 322, 359.

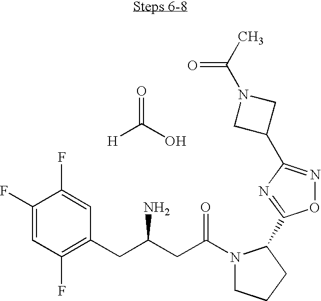

### (R)-1-{(S)-2-[3-(1-Acetyl-azetidin-3-yl)[1,2,4]oxadiazol-5-yl]-pyrrolidin-1-yl}-3-amino-4-(2,4,5-trifluoro-phenyl)-butan-1-one formate salt

The title compound can be prepared by employing the deprotection, peptide coupling and deprotection sequence described for example 12, steps 4-6 with (S)-2-[3-(1-acetylazetidin-3-yl)-[1,2,4]oxadiazol-5-yl]-pyrrolidine-1-carboxylic acid tert-butyl ester from step 5.

LC/MS (III): rt 5.42; m/z 452 [M+H]⁺, 474 [M+Na]⁺.

¹H-NMR (300 MHz, DMSO-d₆) δ=1.73-1.79 (t, 3H, CH₃), 1.96-2.08 (m, 3H, CH, CH₂), 2.20-2.34 (m, 2H, CH₂), 2.59-2.68 (m, 2H, CH₂), 2.86-2.87 (m, 2H, CH₂), 3.50-3.69 (m, 3H, CH, CH₂), 3.87-3.99 (m, 2H, 2×CH₂), 4.17-4.21 (m, 2H, CH₂), 4.47-4.48 (m, 1H, CH), 5.19-5.21 (m, 1H, CH), 7.46-7.58 (m, 2H, aryl-H), 8.14 (s, 1H, HCOOH).

### Example 24

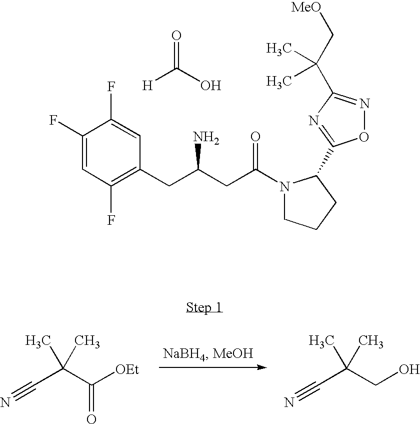

### 3-Hydroxy-2,2-dimethyl-propionitrile

2.06 g (14.8 mmol, 1.00 eq) of 1-cyano-isopropanecarbocylic acid ethyl ester are dissolved in 50 mL of methanol and cooled to 0° C. Then 0.56 g (14.8 mmol, 1.00 eq) of sodium borohydride are added in small portions and the reaction mixture is stirred at room temperature for 3 h. Afterwards the reaction is quenched with 50 mL of saturated sodium bicarbonate solution and treated with 20 mL of water. This mixture is extracted 3 times with diethyl ether, the combined organic extracts are dried with sodium sulphate and the solvent is evaporated. The crude product is used without further purification.

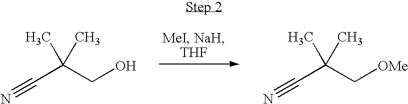

### 3-Methoxy-2,2-dimethyl-propionitrile

0.65 (6.56 mmol, 1.00 eq) of crude 3-hydroxy-2,2-dimethyl-propionitrile from step 1 are dissolved in 10 mL of tetrahydrofuran and added to a stirred mixture of 0.18 g (7.54 mmol, 1.15 eq) of sodium hydride in tetrahydrofuran. The resulting mixture is stirred for 30 min at room temperature and afterwards a solution of 0.47 mL (7.54 mmol, 1.15 g) of methyliodide in 10 mL of tetrahydrofuran is added dropwise. After stirring is continued overnight the reaction mixture is quenched with saturated sodium bicarbonate solution and water. Then the mixture is extracted 3 times with diethyl ether, the combined organic layers are washed with brine, dried with sodium sulphate and the solvent is evaporated. The crude product is used in the next reaction step without purification.

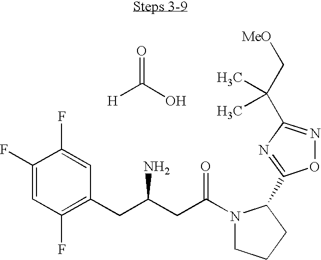

### (R)-3-Amino-1-{(S)-2-[3-(2-methoxy-1,1-dimethyl-ethyl)-[1,2,4]oxadiazol-5-yl]-pyrrolidin-1-yl}-4-(2,4,5-trifluoro-phenyl)-butan-1-one formate salt

The title compound can be prepared by employing reaction sequence described for example 12, steps 1-6 with 3-methoxy-2,2-dimethyl-propionitrile from step 2.

LC/MS (IV): rt 5.08; m/z 441 [M+H]⁺, 463 [M+Na]⁺.

¹H-NMR (400 MHz, DMSO-d₆) δ=1.20-1.26 (dd, 6H, 2×CH₃), 1.90-2.02 (m, 3H, CH, CH₂), 2.27-2.31 (m, 2H, CH₂), 2.60-2.64 (m, 2H, CH₂), 2.86-2.88 (m, 2H, CH₂), 3.15-3.16 (d, 3H, CH₃), 3.34-3.66 (m, 4H, 2×CH₂), 5.15-5.46 (m, 1H, CH), 7.46-7.58 (m, 2H, aryl-H), 8.12 (s, 1H, HCOOH).

### Example 25

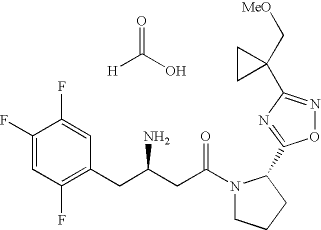

### (R)-3-Amino-1-{(S)-2-[3-(1-methoxymethyl-cyclopropyl)-[1,2,4]oxadiazol-5-yl]-pyrrolidin-1-yl}-4-(2,4,5-trifluoro-phenyl)-butan-1-one formate salt

The title compound can be prepared according to example 24 by using 1-cyanocyclopropanecarboxylic acid ethyl ester in step 1.

LC/MS (I): rt 2.34; m/z 439 [M+H]⁺.

¹H-NMR (400 MHz, DMSO-d₆) δ=1.05-1.15 (m, 4H, 2×CH₂), 1.88-2.01 (m, 3H, CH, CH₂), 2.26-2.29 (m, 1H, CH), 2.55-2.61 (m, 2H, CH₂), 2.80-2.81 (m, 2H, CH₂), 3.21-3.24 (d, 3H, CH₃), 3.49-3.61 (m, 5H, CH, 2×CH₂), 5.10-5.41 (m, 1H, CH), 7.43-7.55 (m, 2H, aryl-H), 8.15 (s, 1H, HCOOH).

### Example 26

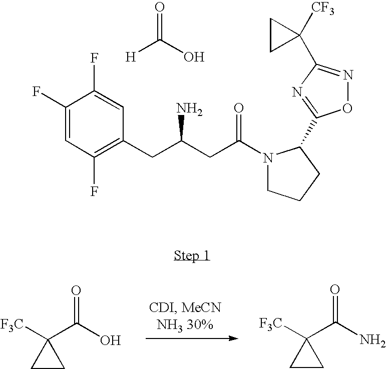

### 1-Trifluoromethyl-cyclopropanecarboxylic acid amide

To a solution of 500 mg (3.25 mmol) 1-trifluoromethyl-cyclopropanecarboxylic acid dissolved in 5 mL acetonitrile 631 mg (3.89 mmol) of CDI are added and the reaction mixture is stirred at room temperature for 2 h. After addition of 3 mL of aqueous ammonium hydroxide (30%) the mixture is stirred overnight. Then, the reaction mixture is diluted by addition of 20 mL ethyl acetate, the organic layer is washed twice with 20 mL of brine and 20 mL of 1M aqueous hydrochloric acid and is dried over sodium sulphate. The solvent is removed to yield the title compound.

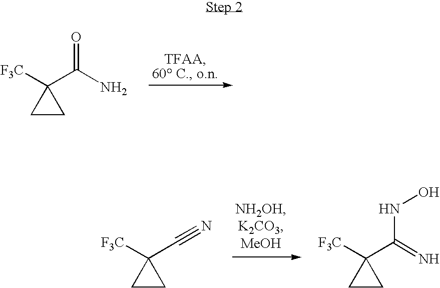

### N-Hydroxy-1-trifluoromethyl-cyclorpropanecarboxamidine

To a solution of 70 mg (0.46 mmol) 1-trifluoromethyl-cyclopropanecarboxylic acid amide (Step 1), dissolved in 1 mL tetrahydrofuran, are added 318 μL (2.29 mmol) of trifluoromethylcarboxylic acid anhydride at ambient temperature. The mixture is stirred at 60° C. overnight, then 568 mg (4.12 mmol) of potassium carbonate, 95 mg (1.37 mmol) of hydroxylamine and 5 mL methanol are added. The mixture is stirred at 65° C. overnight, filtered and removal of the solvent under reduced pressure afforded the title compound which is used in the next step without further purification.

LC/MS (II): 1-60 gradient rt 1.06; m/z 169 [M+H]⁺.

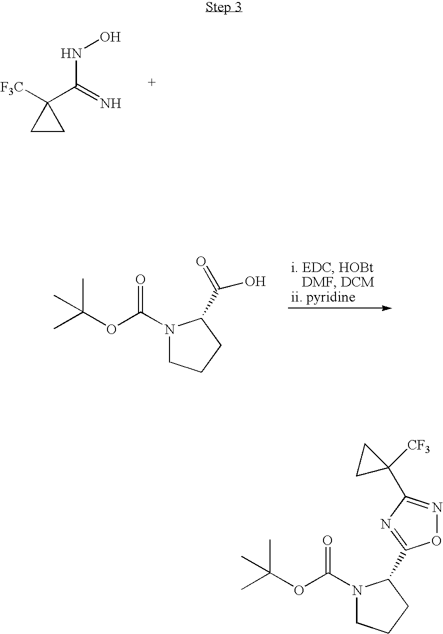

### (S)-2-[3-(1-Trifluoromethyl-cyclopropyl)-[1,2,4]oxadiazol-5-yl]-pyrrolidine-1-carboxylic acid tert-butyl ester

270 mg (1.61 mmol, 1.00 eq) N-hydroxy-1-trifluoromethyl-cyclopropanecarboxamidine (Step 2), 369 mg (1.93 mmol, 1.20 eq) of 1-ethyl-3-(3-dimethylaminopropyl)carbodiimide hydrochloride and 260 mg (1.93 mmol, 1.20 eq) 1-hydroxybenzoltriazole are dissolved in 2 mL of N,N-dimethylformamide. 415 mg (1.93 mmol, 1.20 eq) (S)pyrrolidine-1,2-dicarboxylic acid 1-tert-butyl ester and 249 μL (1.93 mmol, 1.20 eq) of N,N-diisopropylethylamine are dissolved in 5 mL of dichloromethane and slowly added to the carboxamidine reaction mixture. After 4 h stirring a ambient temperature the solvent is removed under reduced pressure and the residue is dissolved in 6 mL pyridine. The reaction mixture is stirred at 120° C. overnight. After removal of the solvents under reduced pressure 20 mL ethyl acetate is added. The organic layer is extracted 2×20 mL 5% citric acid and saturated sodium hydrogen carbonate, washed with brine and dried over sodium sulphate. The solvent is removed to yield the title compound.

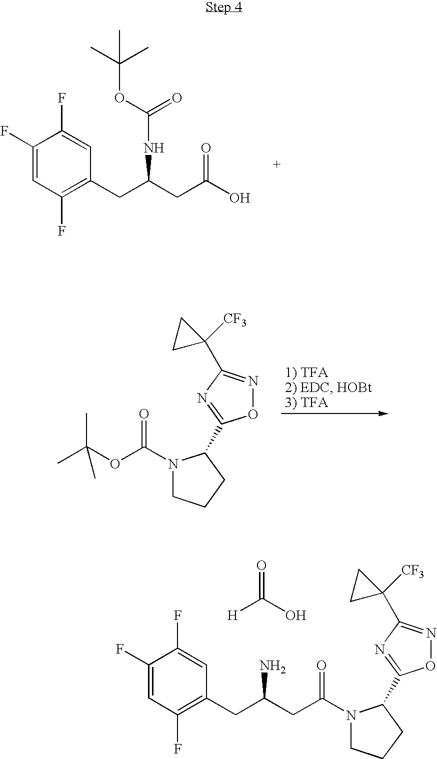

### (R)-3-Amino-1-{(S)-2-[3-(1-trifluoromethyl-cyclopropyl)-[1,2,4]oxadiazol-5-yl]-pyrrolidin-1-yl}-4-(2,4,5-trifluoro-phenyl)-butan-1-one formate salt

201 mg (0.61 mmol) of (R)-3-tert-butoxycarbonylamino-4-(2,4,5-trifluoro-phenyl)-butyric acid, 116 mg (0.601 mmol) of 1-ethyl-3-(3-dimethylaminopropyl)carbodiimide hydrochloride, 81.8 mg (0.61 mmol) of 1-hydroxybenzotriazole and 168 μL (1.21 mmol) triethylamine are diluted in 2 mL of N,N-dimethylformamide and stirred for 30 min at ambient temperature. 140 mg (0.40 mmol) 2-[3-(1-trifluoromethyl-cyclopropyl)-[1,2,4]oxadiazol-5-yl]-pyrrolidine-1-carboxylic acid tert-butyl ester (Step 3) are dissolved in 1 mL dichloromethane and 1 mL of trifluoroacetic acid and stirred for 20 min at ambient temperature. The mixture is diluted with toluene and evaporated under reduced pressure. The residue is dissolved in 2 mL of N,N-dimethylformamide and added dropwise to the preactivated carboxylic acid mixture. The reaction mixture is stirred at room temperature overnight. 20 mL brine are added and the aqueous layer is extracted with 5×20 mL ethyl acetate. The combined organic layers are washed with 2×10 mL 5% aqueous citric acid, saturated sodium bicarbonate solution, brine and dried over sodium sulphate. Removal of the solvent under reduced pressure afforded a residue, which is purified by preparative HPLC (with formic acid as co-eluent) to yield the title compound.

LC/MS (II): 5-70 gradient rt 5.16; m/z 463 [M+H]⁺, 504 [M+H+MeCN]⁺

¹H-NMR (400 MHz, DMSO-d₆) δ=1.38-1.61 (m, 4H, 2×CH₂), 1.90-2.01 (m, 3H, CH, CH₂), 2.25-2.44 (m, 2H, CH₂), 2.65-2.74 (m, 2H, CH₂), 3.21-3.65 (m, 4H, 2×CH₂), 5.13-5.46 (m, 1H, CH), 7.39-7.47 (m, 2H, aryl-H), 8.16 (s, 1H, HCOOH).

### Example 27

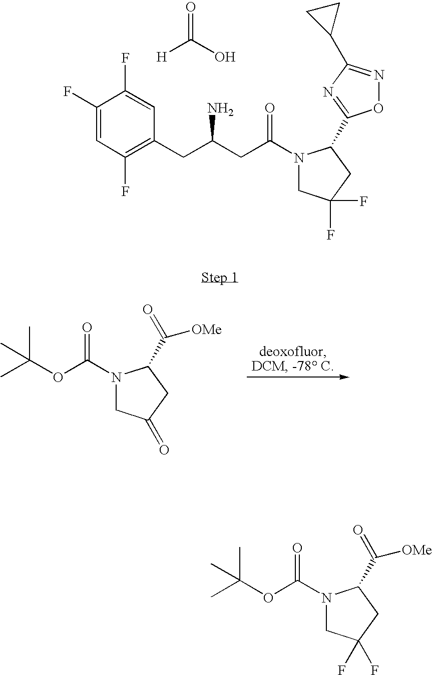

### (S)-4,4-Difluoro-pyrrolidine-1,2-dicarboxylic acid 1-tert-butyl ester 2-methyl ester

130 mg (534 mmol, 1.00 eq) of (S)-4-oxo-pyrrolidine-1,2-dicarboxylic acid 1-tert-butyl ester 2-methyl ester are dissolved in 5 mL of dichloromethane and the solution is cooled to −78° C. Then 394 mL (2.138 mmol, 4.00 eq) of deoxofluor is added dropwise via a syringe. The reaction is stirred overnight, while the temperature is allowed to warm to room temperature. The organic layer is washed with saturated sodium bicarbonate solution, 10% aqueous citric acid solution, water and brine, is dried over sodium sulphate and evaporated under reduced pressure. The crude product is purified by column chromatography on silica gel (cyclo-hexane:ethyl acetate 80:20) to yield the title compound.

LC/MS (I): rt 3.80; m/z 207, 251.

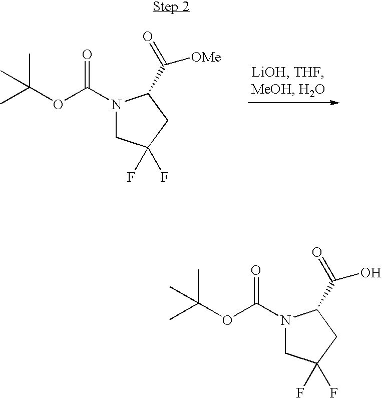

### (S)-4,4-Difluoro-pyrrolidine-1,2-dicarboxylic acid 1-tert-butyl ester

103 mg (388 μmol, 1.00 eq) of (S)-4,4-difluoro-pyrrolidine-1,2-dicarboxylic acid 1-tert-butyl ester 2-methyl ester (Step 1) are dissolved in a mixture of 3 mL tetrahydrofuran and 1 mL methanol. To this solution 179 mg (854 μmol, 2.20 eq) of lithium hydroxide monohydrate dissolved in 1 mL of water is added and the resulting mixture is stirred for 14 h at room temperature. The solvent is removed and the crude product is taken up in water and acidified with 5% aqueous citric acid solution. This aqueous solution is extracted 3 times with dichloromethane and the combined organic extracts are dried with sodium sulphate and the solvent is evaporated to yield the title compound.

¹H-NMR (400 MHz, DMSO-d₆) δ=1.37 (d, 9H, CH₃), 2.36-2.42 (m, 1H, CH), 2.71-2.81 (m, 1H, CH), 3.63-3.75 (m, 1H, CH), 4.21-4.23 (m, 1H, CH).

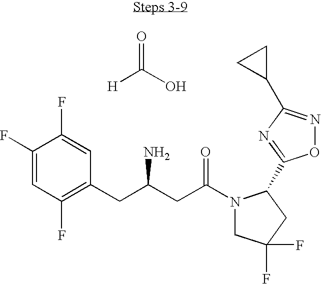

### (R)-3-Amino-1-[(S)-2-(3-cyclopropyl-[1,2,4]oxadiazol-5-yl)-4,4-difluoro-pyrrolidin-1-yl]-4-(2,4,5-trifluoro-phenyl)-butan-1-one formate salt

The title compound can be prepared according to the procedure described for example 12 for steps 1-6 by using cyclopropanecarbonitrile in step 1 and (S)-4,4-difluoro-pyrrolidine-1,2-dicarboxylic acid 1-tert-butyl ester in step 2.

LC/MS (III): rt 7.89; m/z 431 [M+H]⁺, 472 [M+H+MeCN]⁺.

¹H-NMR (400 MHz, DMSO-d₆) δ=0.80-0.89 (m, 2H, CH₂), 1.00-1.11 (m, 2H, CH₂), 2.06-2.12 (m, 1H, CH), 2.43-2.72 (m, 4H, 2×CH₂), 3.01-4.00 (m, 4H, 2×CH₂), 4.15-4.21 (m, 1H, CH), 5.40-5.81 (m, 1H, CH), 7.45-7.48 (m, 2H, aryl-H), 8.21 (s, 1H, HCOOH).

### Example 28

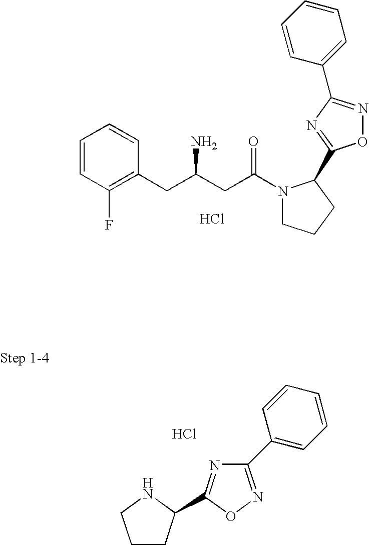

### 3-Phenyl-5-(R)-pyrrolidin-2-yl-[1,2,4]oxadiazole hydrochloride

Steps 1-4 were performed according to the procedures described for example 1 with the exception that (R)-pyrrolidine-1,2-dicarboxylic acid 1-tert-butyl ester was used instead of (S)-pyrrolidine-1,2-dicarboxylic acid 1-tert-butyl ester.

¹H-NMR (300 MHz, DMSO-d₆) δ=2.15-2.65 (m, 4H, 2×CH₂), 3.64 (m, 2H, CH₂), 5.2 (m, 1H, CH), 7.4-7.55 (m, 3H, aryl-H), 8.06 (m, 2H, aryl-H), 10.25 (s, 1H, NH), 11.22 (s, 1H, NH).

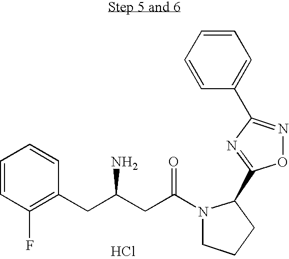

### (R)-3-Amino-4-(2-fluoro-phenyl)-1-[(R)-2-(3-phenyl-[1,2,4]oxadiazol-5-yl)-pyrrolidin-1-yl]-butan-1-one hydrochloride

The title compound was prepared according to steps 5 and 6 of example 1.

¹H-NMR (300 MHz, DMSO-d₆) δ=2.0-3.18 (m, 8H, 4×CH₂), 3.5 (m, 2H, CH₂), 3.73 (m, 1H, CH), 5.18 (m, 1H, CH), 6.9-7.4 (m, 4H, aryl-H), 7.50-7.65 (m, 3H, aryl-H), 7.90-8.02 (m, 2H, aryl-H), 8.3 (m, 3H, NH₃).

### Example 29

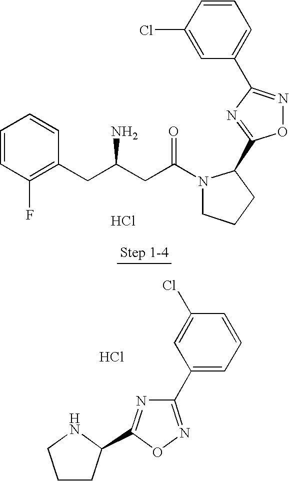

### 3-(3-Chloro-phenyl)-5-(R)-pyrrolidin-2-yl-[1,2,4]oxadiazole hydrochloride

Steps 1-4 were performed according to the procedures described for example 1 with the exception that (R)-pyrrolidine-1,2-dicarboxylic acid 1-tert-butyl ester was used instead of (S)-pyrrolidine-1,2-dicarboxylic acid 1-tert-butyl ester.

¹H-NMR (300 MHz, CDCl₃) δ=2.18 (m, 2H, CH₂), 2.4 (m, 1H, CH), 2.58 (m, 1H, CH), 3.45 (m, 2H, CH₂), 5.2 (m, 1H, CH), 7.6 (dd, 1H, aryl-H), 7.65 (ddd, 1H, aryl-H), 8.02 (ddd, 1H, aryl-H), 8.07 (dd, 1H, aryl-H), 10.3 (s, 2H, NH₂).

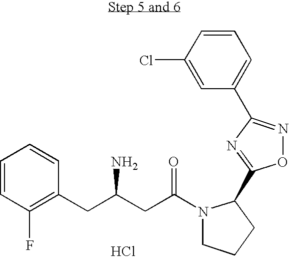

### (R)-3-Amino-1-{(R)-2-[3-(3-chloro-phenyl) [1,2,4]oxadiazol-5-yl)-pyrrolidin-1-yl}-4-(2-fluoro-phenyl)-butan-1-one hydrochloride

The title compound was prepared according to steps 5 and 6 of example 1.

¹H-NMR (300 MHz, CDCl₃) δ=2.2-3.21 (m, 8H, 4×CH₂), 3.5 (m, 2H, CH₂), 3.95 (m, 1H, CH), 5.2 (m, 1H, CH), 6.8-7.5 (m, 6H, aryl-H), 7.9 (m, 1H, aryl-H), 8.1 (dd, 1H, aryl-H), 8.8 (m, 3H, NH₃).

### Example 30

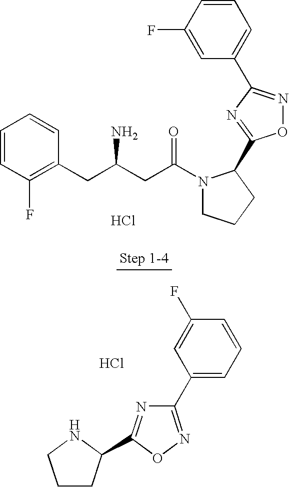

### 3-(3-Fluoro-phenyl)-5-(R)-pyrrolidin-2-yl-[1,2,4]oxadiazole hydrochloride

Steps 1-4 were performed according to the procedures described for example 1 with the exception that (R)-pyrrolidine-1,2-dicarboxylic acid 1-tert-butyl ester was used instead of (S)-pyrrolidine-1,2-dicarboxylic acid 1-tert-butyl ester.

¹H-NMR (300 MHz, CDCl₃) δ=2.23 (m, 2H, CH₂), 2.45-2.62 (m, 2H, CH₂), 3.65 (m, 2H, CH₂), 5.2 (m, 1H, CH), 7.21 (m, 1H, aryl-H), 7.43 (m, 1H, aryl-H), 7.75 (m, 1H, aryl-H), 7.86 (m, 1H, aryl-H), 10.35 (s, 1H, NH), 11.2 (s, 1H, NH).

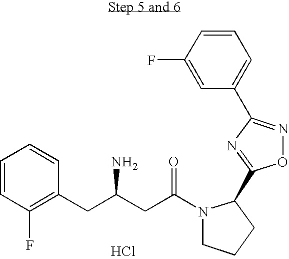

### (R)-3-Amino-1-{(R)-2-[3-(3-fluoro-phenyl) [1,2,4]oxadiazol-5-yl)-pyrrolidin-1-yl}-4-(2-fluoro-phenyl)-butan-1-one hydrochloride

The title compound was prepared according to steps 5 and 6 of example 1.

¹H-NMR (300 MHz, DMSO-d₆) δ=2.0-3.58 (m, 8H, 4×CH₂), 3.6 (m, 2H, CH₂), 3.77 (m, 1H, CH), 5.17 (m, 1H, CH), 8.2 (m, 3H, NH₃), 7.33-7.41 (m, 4H, aryl-H), 7.46 (m, 1H, aryl-H), 7.63 (m, 1H, aryl-H), 7.67 (m, 1H, aryl-H), 7.78 (m, 1H, aryl-H).

### Example 31

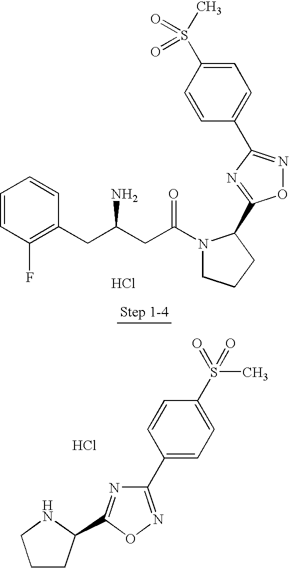

### 3-(4-Methanesulphonyl-phenyl)-5-(R)-pyrrolidin-2-yl-[1,2,4]oxadiazole hydrochloride

Steps 1-4 were performed according to the procedures described for example 1 with the exception that (R)-pyrrolidine-1,2-dicarboxylic acid 1-tert-butyl ester was used instead of (S)-pyrrolidine-1,2-dicarboxylic acid 1-tert-butyl ester.

¹H-NMR (300 MHz, CDCl₃+DMSO-d₆) δ=2.10-2.65 (m, 4H, 2×CH₂). 3.21 (s, 3H, CH₃), 3.46 (m, 2H, CH₂), 5.2 (m, 1H, CH), 8.15-8.3 (m, 4H, aryl-H), 10.0-11.0 (s, 2H, NH₂).

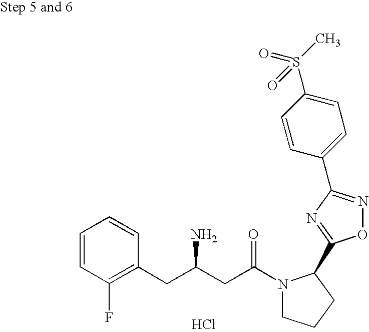

### (R)-3-Amino-4-(2-fluoro-phenyl)-1-{(R)-2-[3-(4-methanesulphonyl-phenyl) [1,2,4]oxadiazol-5-yl]-pyrrolidin-1-yl}-butan-1-one hydrochloride

The title compound was prepared according to steps 5 and 6 of example 1.

¹H-NMR (300 MHz, DMSO-d₆) δ=1.9-2.35 (m, 4H, 2×CH₂), 2.6-3.1 (m, 4H, 2×CH₂), 3.3 (s, 1H, CH₃), 3.6 (m, 1H, CH), 3.7 (m, 2H, CH₂), 5.2 (m, 1H, CH), 7.4-7.1 (m, 4H, aryl-H), 8.08-8.25 (m, 4H, aryl-H), 8.4 (m, 3H, NH₃).

### Example 32

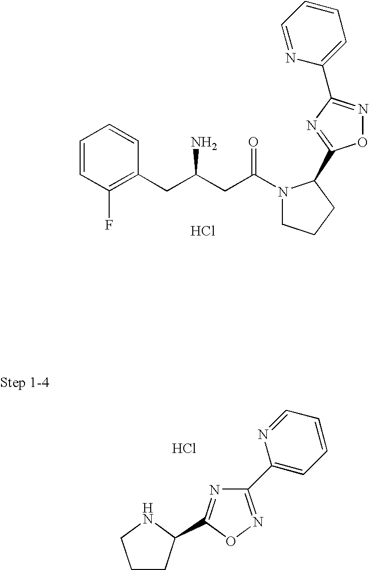

### 2-(5-(R)-Pyrrolidin-2-yl-[1,2,4]oxadiazol-3-yl)-pyridine hydrochloride

Steps 1-4 were performed according to the procedures described for example 1 with the exception that (R)-pyrrolidine-1,2-dicarboxylic acid 1-tert-butyl ester was used instead of (S)-pyrrolidine-1,2-dicarboxylic acid 1-tert-butyl ester.

¹H-NMR (300 MHz, CDCl₃+DMSO-d₆) δ=2.21 (m, 2H, CH₂), 2.42 (m, 1H, CH), 2.6 (m, 1H, CH), 3.44 (m, 2H, CH₂), 5.22 (m, 1H, CH), 7.6 (ddd, 1H, py-H), 8.02 (ddd, 1H, py-H), 8.16 (ddd, 1H, py-H), 8.78 (dd, 1H, py-H), 10.5 (s, 2H, NH₂).

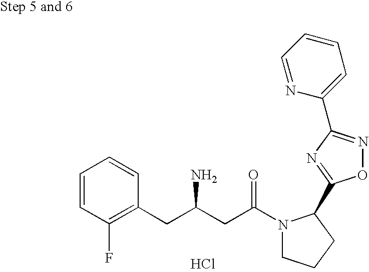

### (R)-3-Amino-4-(2-fluoro-phenyl)-1-[(R)-2-(3-pyridin-2-yl-[1,2,4]oxadiazol-5-yl)pyrrolidin-1-yl]-butan-1-one hydrochloride

The title compound was prepared according to steps 5 and 6 of example 1.

¹H-NMR (300 MHz, DMSO-d₆) δ=2.0 (m, 2H, NH₂), 1.9-2.8 (m, 8H, 4×CH₂), 3.6 (m, 2H, CH₂), 3.8 (m, 1H, CH), 5.22 (m, 1H, CH), 6.9-7.4 (m, 4H, aryl-H), 7.6 (m, 1H, py-H), 7.82-8.2 (m, 2H, py-H), 8.8 (dd, 1H, py-H).

### Example 33

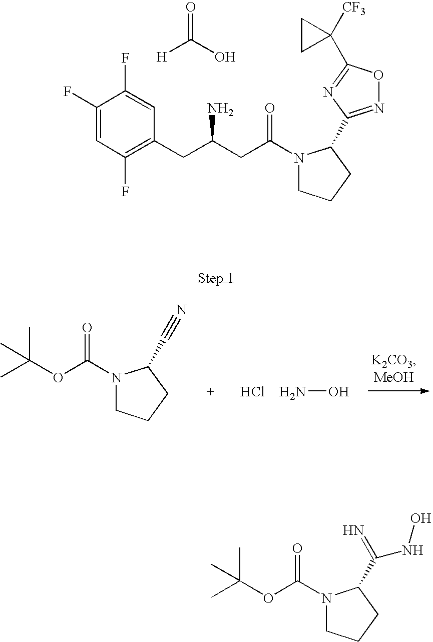

### (S)-2-(N-Hydroxycarbamimidoyl)-pyrrolidine-1-carboxylic acid tert-butyl ester

10.0 g (51.0 mmol, 1.00 eq) of (S)-2-cyano-pyrrolidine-1-carboxylic acid tert-butyl ester are dissolved in 250 mL of methanol. Then 10.6 g (76.4 mmol, 1.50 eq) of potassium carbonate followed by 7.08 g (101 mmol, 2.00 eq) of hydroxylamine hydrochloride are added, and the reaction mixture is refluxed for 8 h. Afterwards the solvent is evaporated, and the residue is taken up in a mixture of water and dichloromethane. The organic layer is separated and extracted three times with 5% citric acid solution. The combined aqueous extracts are treated with saturated sodium bicarboante solution until a basic pH is reached, and are extracted three times with a 3:1 mixture of chloroform and iso-propanol. The combined organic extracts are dried with sodium sulphate, filtered and evaporated under reduced pressure. The amidoxime product could be isolated in form of a colorless solid.

LC/MS (I): rt 1.52; m/z 230 [M+H]⁺, 271 [M+MeCN]⁺.

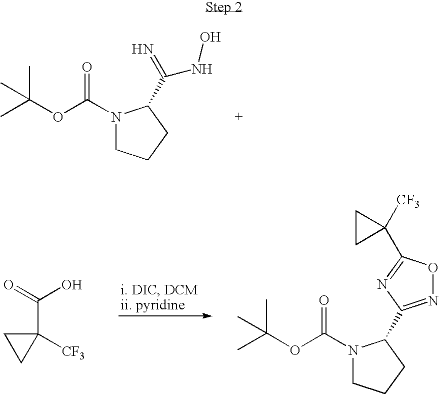

### (S)-2-[5-(1-Trifluoromethyl-cyclopropyl)-[1,2,4]oxadiazol-3-yl]-pyrrolidine-1-carboxylic acid tert-butyl ester

To a solution of 8.10 g (35.3 mmol, 1.00 eq) of (S)-2-(N-hydroxycarbamimidoyl)pyrrolidine-1-carboxylic acid tert-butyl ester and 5.99 g (38.9 mmole, 1.10 eq) of 1-trifluoromethylcyclopropane-1-carboxylic acid in 350 mL of dichloromethane, 6.62 mL (42.4 mmol, 1.20 eq) of 1,3-di-iso-propylcarbodiimide is added. The reaction mixture is stirred at room temperature, and after 3 h the solvent is evaporated. The residue is taken up in 350 mL of pyridine and refluxed for 8 h. Then the pyridine is evaporated and the residue is subjected to column chromatography (silica gel, eluent: cyclohexane:ethyl acetate=50:50) to afford the title compound.

LC/MS (I): rt 4.31; m/z 248 [M+H]⁺, 289 [M+MeCN]⁺.

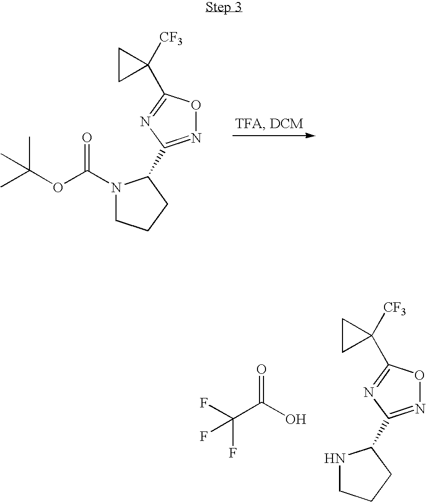

### 3-(S)-Pyrrolidin-2-yl-5-(1-trifluoromethyl-cyclopropyl)-[1,2,4]oxadiazole trifluoroacetic acid salt

10.6 g (31.8 mmol, 1.00 eq) of (S)-2-[5-(1-trifluoromethyl-cyclopropyl)-[1,2,4]-oxadiazol-3-yl]-pyrrolidine-1-carboxylic acid tert-butyl ester are dissolved in 100 mL of dichloromethane and the reaction mixture is cooled to 0° C. with an ice bath. Then 50 mL of trifluoroacetic acid is added and the solution is stirred for 2 h, while the temperature is allowed to warm to rt. Then 10 mL of toluene is added and the solvents are evaporated. The crude product is taken ahead to the next step without any purification.

LC/MS (I): rt 1.76; m/z 289, 333, 348 [M+H]⁺.

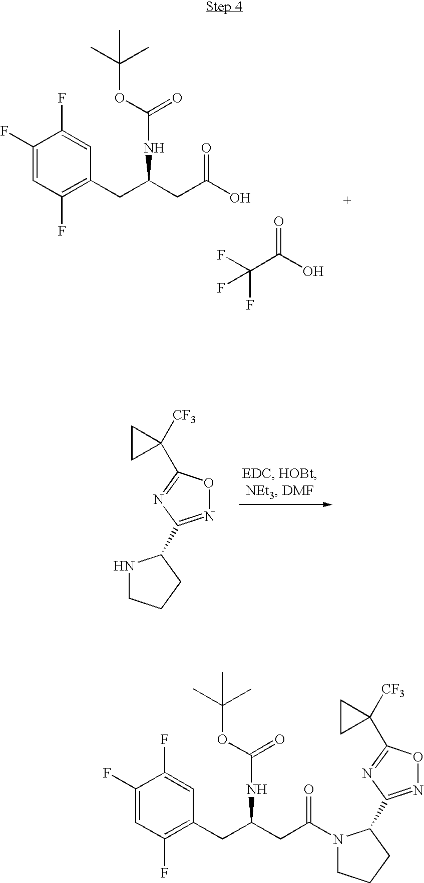

### ((R)-3-Oxo-1-(2,4,5-trifluoro-benzyl)-3-{(S)-2-[5-(1-trifluoromethyl-cyclopropyl)-[1,2,4]oxadiazol-3-yl]-pyrrolidin-1-yl}-propyl)-carbamic acid tert-butyl ester

10.6 g (31.8 mmol, 1.00 eq) of (R)-3-tert-butoxycarbonylamino-4-(2,4,5-trifluorophenyl)-butyric acid are dissolved in 100 mL of N,N-dimethylformamide, and 11.8 mL (79.5 mmol, 2.50 eq) triethylamine followed by 9.14 g (47.7 mmol, 1.50 eq) of 1-hydroxybenzotriazole and 9.14 g (47.7 mmol, 1.50 eq) of 1-ethyl-3-(3-dimethylaminopropyl)carbodiimide hydrochloride are added subsequently. The resulting solution is stirred at rt for 30 min. Then, a solution of the crude product of step 3 and 11.8 mL (79.5 mmol, 2.50 eq) of triethylamine in 100 mL of N,N-dimethylformamide is added. After stirring is continued overnight (10 h) the reaction mixture is diluted with brine and extracted three times with ethyl acetate. The combined organic layers are washed with 5% citric acid solution, saturated sodium bicarbonate solution and brine. The organic layer is dried with sodium sulphate, filtered and evaporated under reduced pressure. The product can be further purified by column chromatography (silica gel, cyclo-hexane:ethyl acetate=50:50). The title compound can be isolated in form of a colorless solid.

LC/MS (I): rt 4.61; m/z 463, 507, 563 [M+H]⁺, 585 [M+Na]⁺.

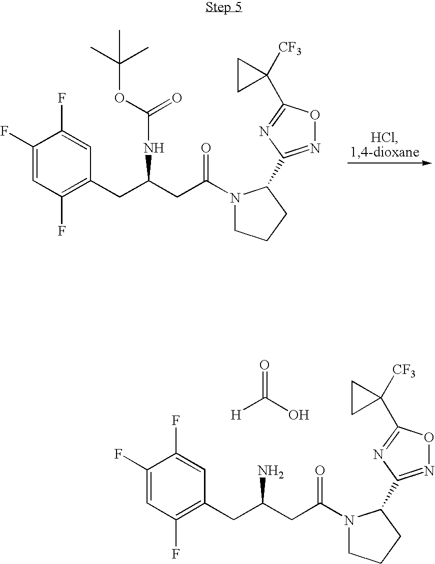

### (R)-3-Amino-1-[(S)-2-[5-(1-trifluoromethyl-cyclopropyl)-[1,2,4]oxadiazol-3-yl]-pyrrolidin-1-yl]-4-(2,4,5-trifluoro-phenyl)-butan-1-one hydrochloride

15.5 g (27.6 mmol, 1.00 eq) of ((R)-3-oxo-1-(2,4,5-trifluoro-benzyl)-3-{(S)-2-[5-(1-trifluoromethyl-cyclopropyl)-[1,2,4]oxadiazol-3-yl]-pyrrolidin-1-yl}-propyl)-carbamic acid tert-butyl ester are dissolved in 200 mL of 1,4-dioxane and at 0° C. a 4M solution of hydrochloric acid in 1,4-dioxane is added. The reaction mixture is stirred for 6 h, while it is allowed to warm to rt. Then the solvent is evaporated and the residue is dried in vacuum. The final compound is isolated as a colourless solid which is purified by preparative HPLC.

LC/MS (III): rt 8.36; m/z 463 [M+H]⁺ 485 [M+Na]⁺, 504 [M+MeCN]⁺.

¹H-NMR (400 MHz, DMSO-d₆) δ=1.65-1.81 (m, 4H, 2×CH₂), 1.83-2.01 (m, 4H, 2×CH, CH₂), 2.20-2.27 (m, 1H, CH), 2.76-2.80 (m, 2H, CH₂), 3.40-3.62 (m, 4H, 2×CH₂), 5.08-5.25 (m, 1H, CH), 7.40-7.52 (m, 2H, aryl-H), 8.21 (s, 1H, HCOOH)

### Example 34

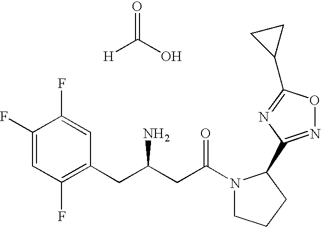

### (R)-3-Amino-1-[(S)-2-(5-cyclopropyl-[1,2,4]oxadiazol-3-yl)-pyrrolidin-1-yl]-4-(2,4,5-trifluoro-phenyl)-butan-1-one formate salt

The title compound can be prepared according to the procedure described for example 33 for steps 1-5 by using cyclopropanecarboxylic acid in step 2.

LC/MS (III): rt 6.78; m/z 395 [M+H]⁺, 436 [M+H+MeCN]⁺.

¹H-NMR (400 MHz, DMSO-d₆) δ=1.00-1.09 (m, 2H, CH₂), 1.18-1.28 (m, 2H, CH₂), 1.82-2.05 (m, 3H, CH, CH₂), 2.16-2.33 (m, 2H, CH₂), 2.42-2.77 (m, 3H, CH, CH₂), 3.41-3.51 (m, 4H, 2×CH₂), 5.00-5.15 (m, 1H, CH), 7.45-7.51 (m, 2H, aryl-H), 8.19 (s, 1H, HCOOH).

### Example 35

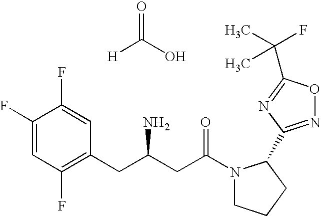

### (R)-3-Amino-1-{(S)-2-[5-(1-fluoro-1-methyl-ethyl)-[1,2,4]oxadiazol-3-yl]-pyrrolidin-1-yl}-4-(2,4,5-trifluoro-phenyl)-butan-1-one

The title compound can be prepared according to the procedure described for example 33 for steps 1-5 by using 2-fluoro-2-methyl-propionic acid in step 2.

LC/MS (III): rt 7.16; m/z 415 [M+H]⁺, 437 [M+Na]⁺, 456 [M+H+MeCN]⁺.

¹H-NMR (400 MHz, DMSO-d₆) δ=1.72-1.82 (dd, 6H, 2×CH₃), 1.82-2.00 (m, 2H, CH₂), 2.21-2.26 (m, 2H, CH₂), 2.70-2.78 (m, 2H, CH₂), 2.85-2.87 (m, 2H, CH₂), 3.47-3.63 (m, 3H, CH, CH₂), 5.10-5.30 (m, 1H, CH), 7.48-7.52 (m, 2H, aryl-H), 8.13 (s, 1H, HCOOH).

### Example 36

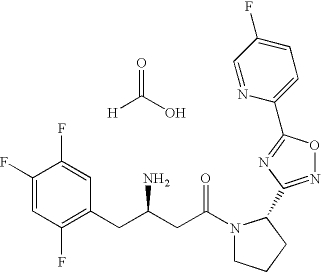

### (R)-3-Amino-1-{(S)-2-[5-(5-fluoro-pyridin-2-yl)-[1,2,4]oxadiazol-3-yl]-pyrrolidin-1-yl}-4-(2,4,5-trifluoro-phenyl)-butan-1-one formate salt

The title compound can be prepared according to the procedure described for example 33 for steps 1-5 by using 5-fluoro-pyridine-2-carboxylic acid in step 2.

LC/MS (III): rt 7.93; m/z 450 [M+H]⁺, 491 [M+H+MeCN]⁺.

¹H-NMR (400 MHz, DMSO-d₆) δ=1.94-2.16 (m, 4H, 2×CH₂), 2.24-2.40 (m, 2H, CH₂), 2.57-2.79 (m, 2H, CH₂), 2.85-2.87 (m, 2H, CH₂), 3.37-3.66 (m, 3H, CH, CH₂), 5.17-5.34 (m, 1H, CH), 7.42-7.54 (m, 2H, aryl-H), 7.97-8.05 (m, 1H, py-H), 8.12 (s, 1H, HCOOH), 8.24-8.32 (m, 1H, py-H), 8.81-8.86 (m, 1H, py-H).

### Example 37

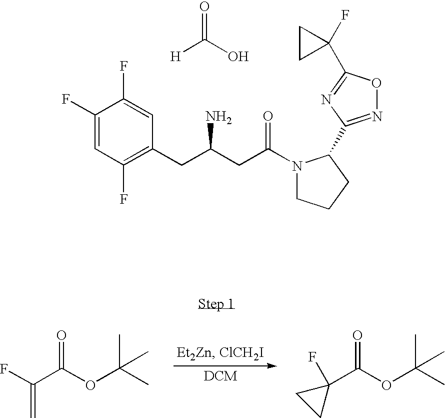

### 1-Fluoro-cyclopropanecarboxylic acid tert-butyl ester

500 mg (3.42 mmol, 1.00 eq) of 2-fluoro-acrylic acid tert-butyl ester are dissolved in 15 mL of dichloromethane. The solution is cooled to −30° C. and 15 mL (16.0 mmol, 4.80 eq) of a 1.1M solution of diethylzinc in toluene followed by 2.5 mL (34.2 mmol, 10.0 eq) of chloroiodomethane is added. The reaction mixture is stirred for 3 h between −40 and −25° C., and 2 h as it warms to 10° C. To the reaction mixture saturated ammonium chloride solution is added carefully. The solution is stirred for 5 min. and diluted with diethyl ether. After extraction, the organic layer is washed with water and brine, dried with sodium sulphate, filtered and evaporated until 14 mL of organic layer remained in the flask. The solution is taken in the next step without any purification.

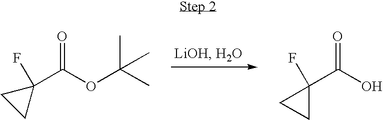

1-Fluoro-cyclopropanecarboxylic acid

To the solution of step 1, 2.50 g (104.0 mmol, 30.0 eq) of lithium hydroxide dissolved in 20 mL of water is added and the reaction mixture is refluxed overnight (16 h). The reaction mixture is cooled to room temperature, acidified with concentrated hydrochloric acid until pH=1 could be obtained. The aqueous layer is extracted three time with diethyl ether and the organic layers are dried with sodium sulphate, filtered and evaporated until 14 mL of organic layer remained in the flask. The solution is taken in the next step without any purification.

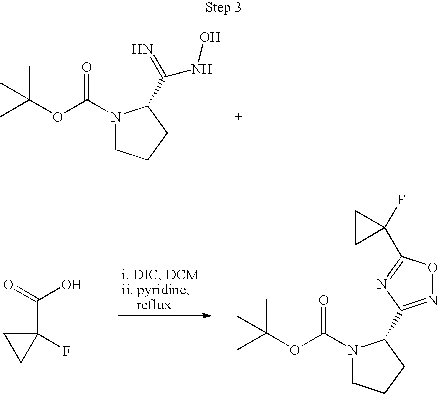

### (S)-2-[5-(1-Fluoro-cyclopropyl)-[1,2,4]oxadiazol-3-yl]-pyrrolidine-1-carboxylic acid tert-butyl ester

To a solution of 430 mg (1.87 mmol, 1.00 eq) of (S)-2-(N-hydroxycarbamimidoyl)pyrrolidine-1-carboxylic acid tert-butyl ester (see example 33, step 1) in 20 mL of dichloromethane, 14 mL of a solution of 1-fluorocyclopropane-1-carboxylic acid (Step 2) and 600 μL (3.74 mmol, 2 eq) of N,N′-diisopropylcarbodiimide is added. The reaction mixture is stirred at room temperature, and after 24 h the solvent is evaporated. The residue is purified by flash chromatography (silica gel, eluent: dichloromethane:methanol=1:0 to 1:1). 70 mg (0.22 mmol) of the O-acyl amidoxime is dissolved in 5.00 mL of pyridine and the solution is heated at 117° C. for 3 h. The reaction mixture is diluted with dichloromethane and washed with 5% citric acid solution, saturated sodium bicarbonate solution and brine. The organic layer is dried with sodium sulphate, filtered and evaporated under reduced pressure. The crude product is taken in the next step without any purification.

LC/MS (I): rt 3.50; m/z 241, 297 [M+H]⁺, 360.

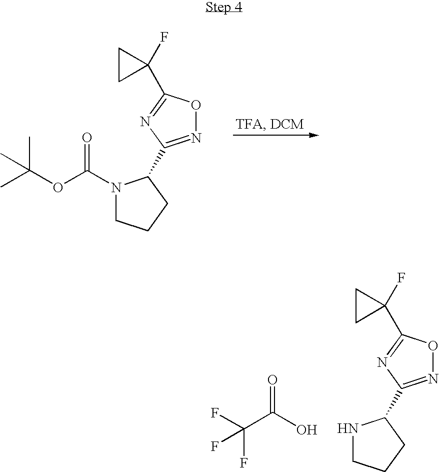

### 3-(S)-Pyrrolidin-2-yl-5-(1-fluoro-cyclopropyl)-[1,2,4]oxadiazole trifluoro-acetic acid salt

To a solution of 45 mg (0.15 mmol, 1.00 eq) of (S)-2-[5-(1-fluoro-cyclopropyl)-[1,2,4]-oxadiazol-3-yl]-pyrrolidine-1-carboxylic acid tert-butyl ester in 2 mL of dichloromethane, 800 μL of trifluoroacetic acid is added. The reaction mixture is stirred at room temperature, and after 2 h the solvent is evaporated. Then 1 mL of toluene is added and the solvent is evaporated. The crude product is taken in the next step without any purification.

LC/MS (I): rt 1.42; m/z 197 [M+H]⁺, 238 [M+MeCN]⁺.

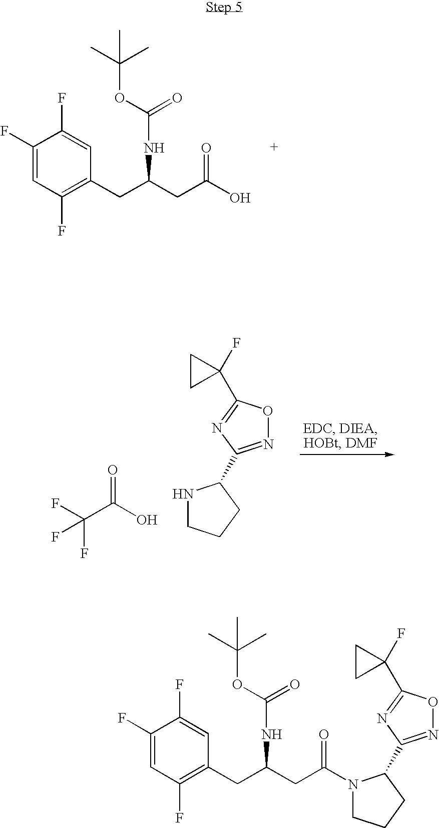

### ((R)-3-Oxo-1-(2,4,5-trifluoro-benzyl)-3-{(S)-2-[5-(1-fluoro-cyclopropyl)-[1,2,4]oxadiazol-3-yl]-pyrrolidin-1-yl}-propyl)-carbamic acid tert-butyl ester

42 mg (0.12 mmol, 1.00 eq) of (R)-3-tert-butoxycarbonylamino-4-(2,4,5-trifluorophenyl)-butyric acid are dissolved in 1 mL of N,N-dimethylformamide, and 67 μL (0.50 mmol, 4.00 eq) diisopropylethylamine followed by 25.7 mg (0.19 mmol, 1.50 eq) of 1-hydroxybenzotriazole and 36 mg (0.19 mmol, 1.50 eq) of 1-ethyl-3-(3-dimethylaminopropyl)carbodiimide hydrochloride are added subsequently. The resulting solution is stirred at rt for 5 min. Then, a solution of the crude product of step 4 in 1 mL of N,N-dimethylformamide is added. After stirring is continued overnight (18 h) and the reaction mixture is diluted with diethyl ether and washed with 1 M hydrochloric acid solution, water and brine. The organic layer is dried with sodium sulphate, filtered and evaporated under reduced pressure. The product is purified by column chromatography (silica gel, cyclo-hexane:ethyl acetate=3:1 to 0:1) to give the title compound.

LC/MS (I): rt 4.14; m/z 412, 456, 512 [M+H]⁺, 534 [M+Na]⁺.

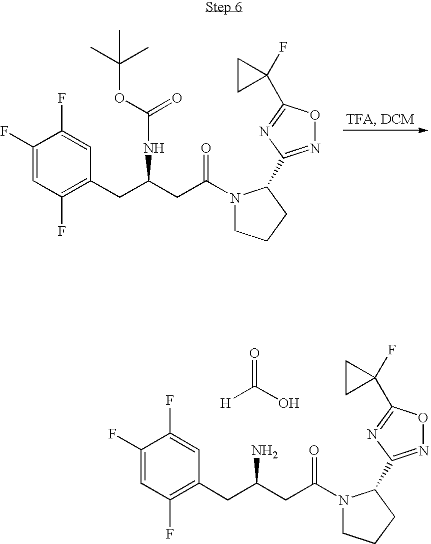

### (R)-3-Amino-1-{(S)-2-[5-(1-fluoro-cyclopropyl)-[1,2,4]oxadiazol-3-yl]-pyrrolidin-1-yl}-4-(2,4,5-trifluoro-phenyl)-butan-1-one formate salt

To a solution of 40 mg (0.07 mmol, 1.00 eq) of ((R)-3-oxo-1-(2,4,5-trifluoro-benzyl)-3-{(S)-2-[5-(1-fluoro-cyclopropyl)-[1,2,4]oxadiazol-3-yl]-pyrrolidin-1-yl}-propyl)-carbamic acid tert-butyl ester (Step 5) in 2 mL of dichloromethane, 600 μL of trifluoroacetic acid is added. The reaction mixture is stirred at room temperature, and after 1 h the solvent is evaporated. Then 1 mL of methanol is added, the solution is filtered and the solvent evaporated. The crude product is purified by preparative LC/MS to yield the title compound.

LC/MS (I): rt 2.34; m/z 412 [M+H]⁺, 453 [M+H+MeCN]⁺.

¹H-NMR (400 MHz, DMSO-d₆) δ=1.08-1.15 (m, 4H, 2×CH₂ propyl), 1.79-2.31 (m, 5H, CH, CH₂), 2.62-2.85 (m, 2H, CH₂), 3.26-3.71 (m, 4H, CH, CH₂), 4.84-4.93 (m, 1H, CH), 7.40-7.54 (m, 2H, aryl-H), 8.14 (s, 1H, HCOOH), 8.15-8.34 (dd, 1H, NH).

### Example 38

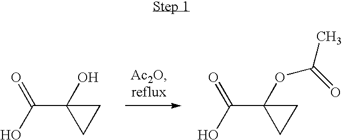

### 1-Acetoxy-cyclopropanecarboxylic acid

100 mg (1.00 mmol, 1.00 eq) of 1-hydroxy-cyclopropanecarboxylic acid is heated to reflux in 1.00 mL of acetic anhydride for 90 min. The mixture is cooled to 80° C. and water is added. The solvent is removed under reduced pressure and the crude product is recrystallized from diethyl ether to yield the title compound.

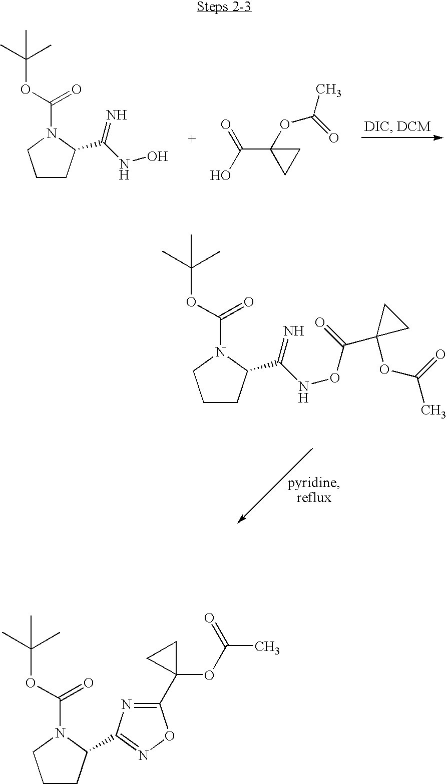

### (S)-2-[5-(1-Acetoxy-cyclopropyl)-[1,2,4]oxadiazol-3-yl]-pyrrolidine-1-carboxylic acid tert-butyl ester

16 mg (0.11 mmol, 1.10 eq) of 1-acetoxy-cyclopropanecarboxylic acid and 23 mg (0.10 mmol, 1.00 eq) of (S)-2-(N-hydroxycarbamimidoyl)-pyrrolidine-1-carboxylic acid tert-butyl ester (example 33, step 1) are dissolved in 4 mL of dichloromethane and 18.9 μL (0.12 mmol, 1.20 eq) N,N′-diisopropylcarbodiimide are added. The resulting mixture is stirred for 3 h at room temperature, afterwards the solvent is evaporated and the residue is heated to reflux in 2 mL of pyridine for 12 h. The solvent is again evaporated and the crude product is purified by column chromatography on silica gel (cyclohexane:ethyl acetate 50:50) to yield the title compound.

LC/MS (I): rt 3.95; m/z 279, 282, 360 [M+Na]⁺, 401 [M+Na⁺ MeCN]⁺.

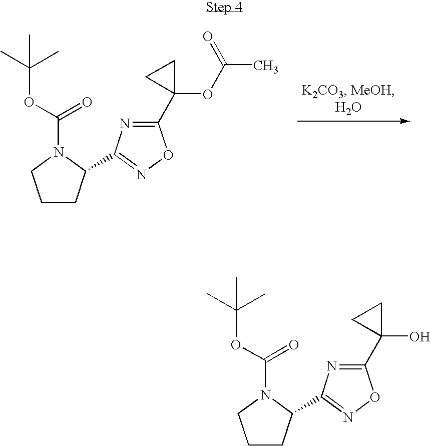

### (S)-2-[5-(1-Hydroxy-cyclopropyl)-[1,2,4]oxadiazol-3-yl]-pyrrolidine-1-carboxylic acid tert-butyl ester

10 mg (0.04 mmol, 1.00 eq) of (S)-2-[5-(1-acetoxy-cyclopropyl)-[1,2,4]oxadiazol-3-yl]-pyrrolidine-1-carboxylic acid tert-butyl ester (Step 3) are dissolved in 1 mL of methanol and 0.25 mL of a 10% aqueous potassium carbonate solution is added. The resulting mixture is stirred for 90 min at room temperature. Afterwards the solvent is evaporated and the crude product is purified by column chromatography on silica gel (cyclohexane:ethyl acetate=50:50) affording the title compound.

LC/MS (I): rt 3.18; m/z 237, 240, 318 [M+Na]⁺, 359 [M+Na⁺ MeCN]⁺.

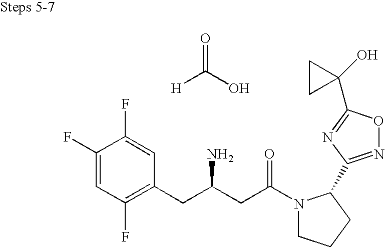

### (R)-3-Amino-1-{(S)-2-[5-(1-hydroxy-cyclopropyl)-[1,2,4]oxadiazol-3-yl]-pyrrolidin-1-yl}-4-(2,4,5-trifluoro-phenyl)-butan-1-one formate salt

The title compound can be prepared by employing the deprotection, peptide coupling and deprotection sequence described for example 33, steps 3-6 with (S)-2-[5-(1-hydroxy-cyclopropyl)-[1,2,4]oxadiazol-3-yl]-pyrrolidine-1-carboxylic acid tert-butyl ester from step 4.

LC/MS (IV): rt 3.09; m/z 411 [M+H]⁺, 433 [M+Na]⁺, 452 [M+MeCN]⁺.

### Example 39

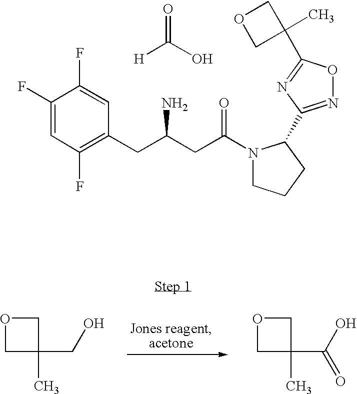

### 3-Methyl-oxetane-3-carboxylic acid

400 μL (4.05 mmol, 1.00 eq) of 3-methyl-3-oxetan-methanol is dissolved in 20 mL of acetone. The solution is cooled to 0° C. and an acidic aqueous solution of chromium trioxide (2.5 g CrO₃ dissolved in a mixture of 2.5 mL of sulphuric acid and 7.5 mL of water) is added dropwise via a dropping funnel until the yellow colour of the reaction mixture persists. After stirring has been continued for 2.5 h at room temperature the reaction is quenched with isopropanol, the mixture is filtered and the filitrate is evaporated. The residue is taken up in dichloromethane, washed with brine, dried over sodium sulphate and the solvent is removed under reduced pressure to yield the title compound.

¹H-NMR (400 MHz, DMSO-d₆) δ=1.46 (s, 3H, CH₃), 4.26 (d, 2H, CH₂), 4.71 (d, 2H, CH₂), 12.7 (s, 1H, COOH).

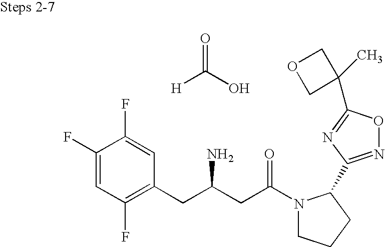

### (R)-3-Amino-1-{(S)-2-[5-(3-methyl-oxetan-3-yl)[1,2,4]oxadiazol-3-yl]-pyrrolidin-1-yl}-4-(2,4,5-trifluoro-phenyl)-butan-1-one formate salt

The title compound can be prepared according to example 33, steps 1-6 by using 3-methyl-oxetane-3-carboxylic acid from step 1 as starting material.

LC/MS (III): rt 6.44; m/z 425 [M+H]⁺, 447 [M+Na]⁺, 466 [M+H+MeCN]⁺.

¹H-NMR (400 MHz, DMSO-d₆) δ=1.69-1.75 (d, 3H, CH₃), 1.87-2.02 (m, 3H, CH, CH₂), 2.19-2.24 (m, 1H, CH), 2.53-2.91 (m, 4H, 2×CH₂), 3.41-3.65 (m, 3H, CH, CH₂), 4.50-4.56 (m, 2H, OCH₂), 4.81-4.87 (m, 2H, OCH₂), 5.10-5.25 (m, 1H, CH), 7.45-7.52 (m, 2H, aryl-H), 8.14 (s, 1H, HCOOH).

### Example 40

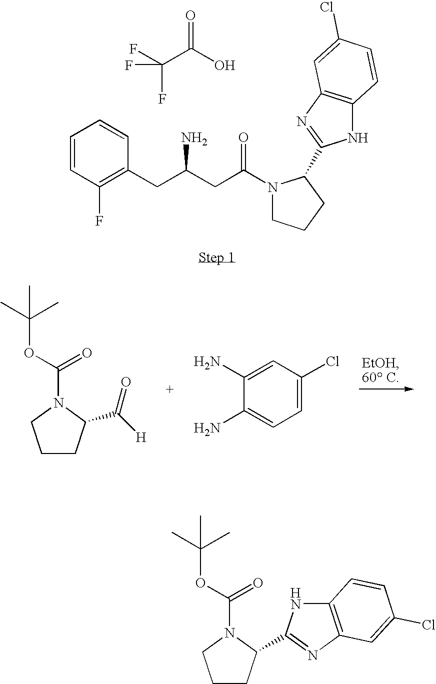

### tert-Butoxy-[(S)-2-(5-chloro-1H-benzoimidazol-2-yl)-pyrrolidin-1-yl]-methanol

100 mg (0.50 mmol, 1.00 eq) of (S)-2-formyl-pyrrolidine-1-carboxylic acid tert-butyl ester and 72.0 mg (0.50 mmol, 1.00 eq) of 2-amino-4-chloroaniline are dissolved in ethanol and heated under an air atmosphere to 60° C. for 2 days. The resulting solution is evaporated under reduced pressure and the crude product is purified by flash chromatography (silica gel, eluent: dichloromethane:methanol=98:2) to yield the title compound.

LC/MS (I): rt 2.09; m/z 322 [M+H]⁺.

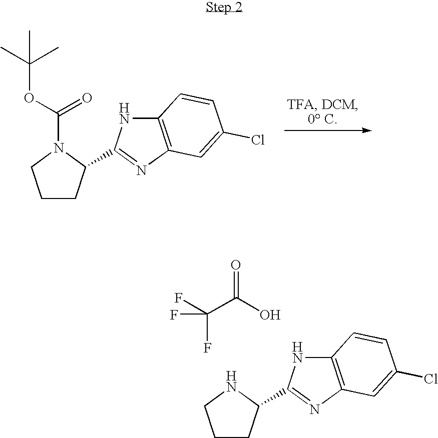

### 5-Chloro-(S)-2-pyrrolidin-2-yl-1H-benzoimidazole trifluoroacetic acid salt

36.4 mg (113 mmol, 1.00 eq) of the compound from step 1 is dissolved in 1.00 mL of dichloromethane and at 0° C. 0.50 mL trifluoroacetic acid is added to the mixture. The solution is allowed to warm to room temperature and after 1 h the solvents are evaporated to yield the title compound. The crude product is used without further purification in step 3.

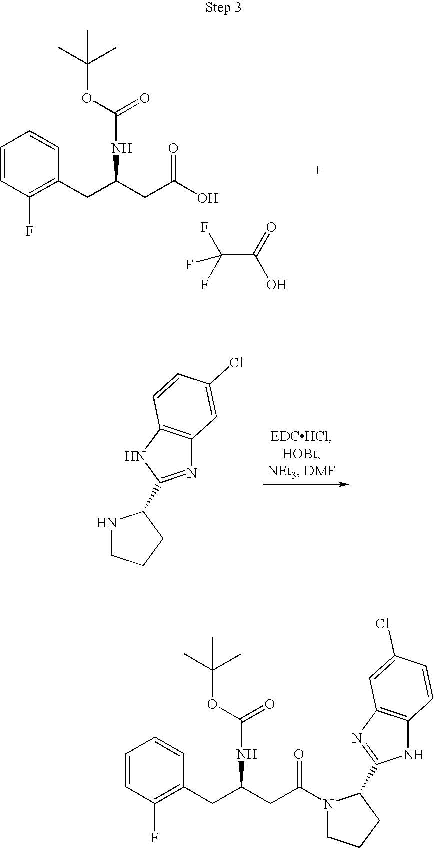

### (R)-[3-[(S)-2-(5-Chloro-1H-benzoimidazol-2-yl)-pyrrolidin-1-yl]-1-(2-fluoro-benzyl)-3-oxo-propyl]-carbamic acid tert-butyl ester

19.1 mg (141 mmol, 1.50 eq) of 1-hydroxybenzotriazole, 47.3 μL (34.3 mg, 339 μmol, 3.60 eq) triethylamine and 27.1 mg (141 mmol, 1.50 eq) of 1-ethyl-3-(3-dimethylaminopropyl)carbodiimide hydrochloride are added to a stirred solution of 28.0 mg (94.0 mmol, 1.00 eq) of (R)-3-tert-butoxycarbonylamino-4-(2-fluoro-phenyl)-butyric acid in 1.00 mL of N,N-dimethylformamide. The reaction mixture is stirred for 15 min. at room temperature. Then a solution of 37.9 mg (113 mmol, 1.20 eq) of the crude deprotected pyrrolidine compound from step 2 in 1.00 mL of N,N-dimethylformamide is added. The resulting solution is stirred for 12 h, then poured into brine and diluted with water. The aqueous mixture is extracted 3 times with ethyl acetate and the combined organic layers are washed with 5% citric acid solution, saturated sodium bicarbonate solution, brine and are dried with sodium sulphate. The solvent is evaporated and the residue is subjected to flash chromatography (silica gel, eluent: cyclo-hexane:ethyl acetate=1:1) to yield the title compound.

LC/MS (I): rt 2.56; m/z 501 [M+H]⁺.

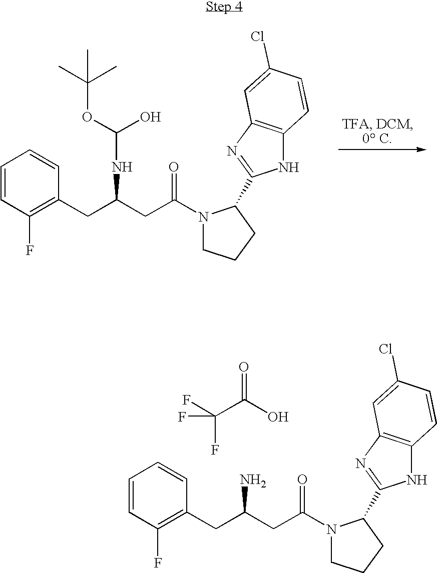

### (R)-3-Amino-1-[(S)-2-(5-chloro-1H-benzoimidazol-2-yl)-pyrrolidin-1-yl]-4-(2-fluorophenyl)-butan-1-one trifluoroacetic acid salt

The purified compound from step 3 is dissolved in 1.00 mL of dichloromethane. The solution is cooled to 0° C. and 0.50 mL of trifluoroacetic acid is added. The reaction mixture is allowed to warm to room temperature and after 30 min. the solvent is evaporated to yield the crude title compound, which is purified by preparative LC/MS.

LC/MS (I): rt 1.91; m/z 401 [M+H]⁺.

¹H-NMR (300 MHz, DMSO-d₆) δ=2.02-2.08 (m, 3H, CH, CH₂), 2.30-2.31 (m, 1H, CH), 2.63 (m, 2H, CH₂), 2.90-3.00 (m, 2H, CH₂), 3.50-3.60 (m, 3H, CH, CH₂), 5.18 (m, 1H, CH), 7.03-7.33 (m, 5H, aryl-H), 7.51-7.59 (m, 2H, aryl-H), 7.92 (m, 3H, NH).

### Example 41

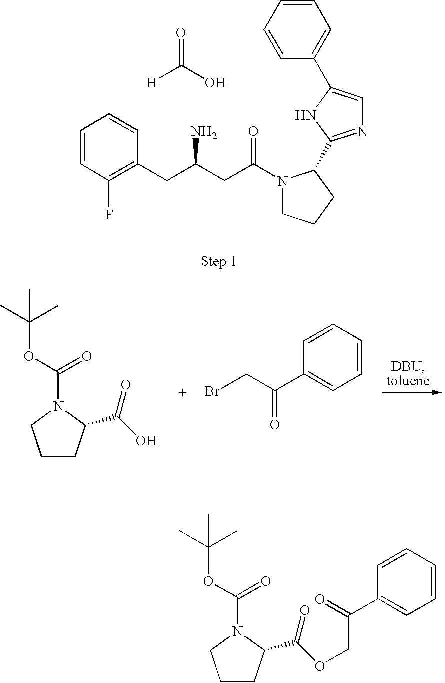

### (S)-Pyrrolidine-1,2-dicarboxylic acid 1-tert-butyl ester 2-(2-oxo-2-phenyl-ethyl) ester

50 mg (0.23 mmol, 1.00 eq) of (S)-pyrrolidine-1,2-dicarboxylic acid 1-tert-butyl ester is dissolved in 1 mL of toluene, and 31.8 mg (0.21 mmol, 0.90 eq) of 1,8-diazabicyclo[5.4.0]-undec-7-ene followed by 34.7 mg (0.17 mmol, 0.80 eq) of 2-bromoacetophenone is added to the solution. After stirring for 2 h at room temperature the reaction mixture is diluted with ethyl acetate and washed with 10% aqueous citric acid solution, saturated sodium bicarbonate solution and brine. The organic layer is dried with sodium sulphate, evaporated to yield the title compound.

LC/MS (I): rt 4.22; m/z 234, 276, 356 [M+Na]⁺, 397 [M+Na⁺ MeCN]⁺.

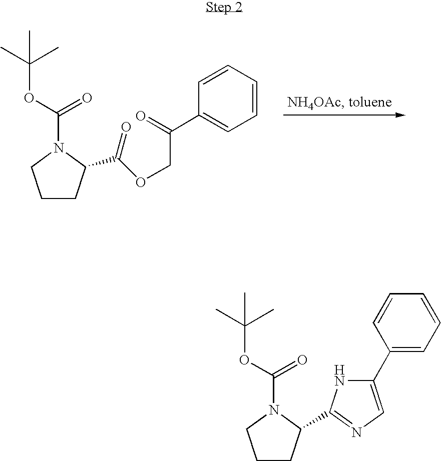

### (S)-2-(5-Phenyl-1H-imidazol-2-yl)-pyrrolidine-1-carboxylic acid tert-butyl ester

116 mg (0.35 mmol, 1.00 eq) of (S)-pyrrolidine-1,2-dicarboxylic acid 1-tert-butyl ester 2-(2-oxo-2-phenyl-ethyl) ester (Step 1) and 268 mg (3.50 mmol, 10.0 eq) of ammonium acetate are dissolved in 3 mL toluene and heated in a bomb tube for 18 h. Afterwards the mixture is filtered and the filtrate is evaporated under reduced pressure. The residue is purified by column chromatography on silica gel (cyclo-hexane:ethyl acetate 50:50) to yield the title compound.

LC/MS (I): rt 2.29; m/z 258, 314 [M+H]⁺, 355 [M+H+MeCN]⁺, 377 [M+Na⁺ MeCN]⁺.

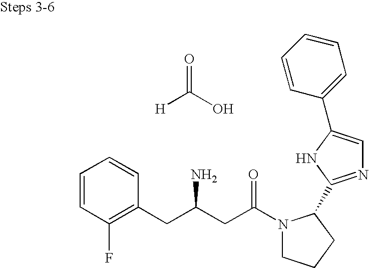

### (R)-3-Amino-4-(2-fluoro-phenyl)-1-((S)-2-{5-[(E)-((Z)-1-propenyl)-buta-1,3-dienyl]-1H-imidazol-2-yl}-pyrrolidin-1-yl)-butan-1-one formate salt

The title compound can be prepared by employing the deprotection, peptide coupling and deprotection sequence described for example 12, steps 4-6 with (S)-2-(5-phenyl-1H-imidazol-2-yl)-pyrrolidine-1-carboxylic acid tert-butyl ester from step 2.

LC/MS (IV): rt 6.59; m/z 393 [M+H]⁺, 415 [M+Na]⁺.

¹H-NMR (400 MHz, DMSO-d₆) δ=1.85-2.33 (m, 5H, CH, 2×CH₂), 2.87-2.91 (m, 2H, CH₂), 3.39-3.43 (m, 2H, CH₂), 3.54-3.58 (m, 2H, CH₂), 4.99-5.08 (m, 1H, CH), 7.13-7.39 (m, 7H, aryl-H), 7.51 (s, 1H, aryl-H), 7.67-7.75 (m, 2H, aryl-H), 8.21 (s, 1H, HCOOH).

### Example 42

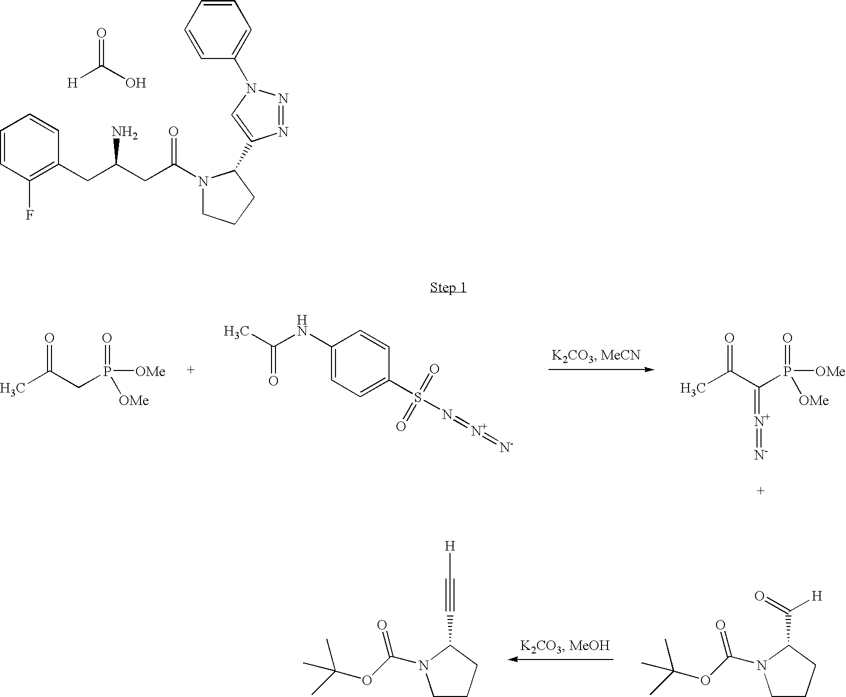

### (S)-2-Ethynyl-pyrrolidine-1-carboxylic acid tert-butyl ester

To a solution of 289 mg (1.20 mmol, 1.20 eq) of 4-acetylamino-benzenesulphonyl azide 416 mg (3.01 mmol, 3.00 eq) of potassium carbonate and 165 μL (1.20 mmol, 1.20 eq) of (2-oxo-propyl)-phosphonic acid dimethyl ester are added and the resulting mixture is stirred for 4 h at room temperature. Afterwards 200 mg (1.00 mmol, 1.00 eq) of (S)-2-formyl-pyrrolidine-1-carboxylic acid tert-butyl ester dissolved in 3 mL methanol is added and the reaction is stirred for 14 h at room temperature. Then the solvent is evaporated and the residue is taken up in a 1:1 mixture of diethyl ether and water. The water phase is separated and extracted 3 times with diethyl ether. The combined organic phases are dried with sodium sulphate, evaporated under reduced pressure and the residue is purified by column chromatography on silica gel (cyclo-hexane:ethyl acetate 90:10) to yield the title compound.

LC/MS (I): rt 5.09; m/z 196 [M+H]⁺, 237 [M+MeCN]⁺.

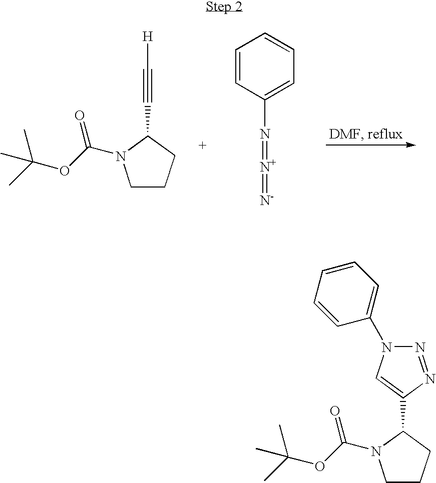

### (S)-2-(1-Phenyl-1H-[1,2,3]triazol-4-yl)-pyrrolidine-1-carboxylic acid tert-butyl ester

104 mg (0.53 mmol, 1.00 eq) of (S)-2-ethynyl-pyrrolidine-1-carboxylic acid tert-butyl ester and 64 mg (0.53 mmol, 1.00 eq) of phenylazide (freshly prepared according to Q. Lin, Y. Tor, Org. Lett. 2003, 5, 14, 2571-2572.) are dissolved in 2 mL of N,N-dimethylformamide, and the mixture is heated to 150° C. for 90 min. After cooling to room temperature the reaction solution is diluted with ethyl acetate and washed with brine, saturated sodium bicarbonate solution, water and brine, again. The organic layer is dried with sodium sulphate, evaporated, and the crude mixture is purified by column chromatography on silica gel (cyclo-hexane:ethyl acetate 70:30) to afford the pure title compound.

LC/MS (I): rt 3.99; m/z 259, 315 [M+H]⁺, 378 [M+Na⁺ MeCN]⁺.

¹H-NMR (400 MHz, DMSO-d₆) δ=1.21-1.38 (d, 9H, CH₃), 1.90-2.27 (m, 4H, 2×CH₂), 3.30-3.51 (m, 4H, 2×CH₂), 4.93-5.00 (m, 1H, CH), 7.45-7.49 (m, 1H, aryl-H), 7.57-7.61 (m, 2H, aryl-H), 7.88-7.91 (m, 2H, aryl-H), 8.63 (d, 1H, triazole-H).

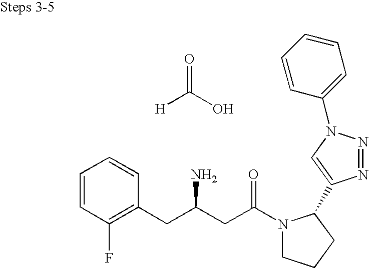

### (R)-3-Amino-4-(2-fluoro-phenyl)-1-((S)-2-{1-[(E)-((Z)-1-propenyl)-buta-1,3-dienyl]-1H-[1,2,3]triazol-4-yl}-pyrrolidin-1-yl)-butan-1-one formate salt

The title compound can be prepared by employing the deprotection, peptide coupling and deprotection sequence described for example 12, steps 4-6 with (S)-2-(1-phenyl-1H-[1,2,3]triazol-4-yl)-pyrrolidine-1-carboxylic acid tert-butyl ester from step 2.

LC/MS (IV): rt 4.34; m/z 394 [M+H]⁺, 416 [M+Na]⁺.

¹H-NMR (400 MHz, DMSO-d₆) δ=1.85-2.05 (m, 3H, CH, CH₂), 2.11-2.31 (m, 1H, CH), 2.55-2.60 (m, 2H, CH₂), 2.65-2.92 (m, 2H, CH₂), 3.34-3.41 (m, 2H, CH₂), 3.56-3.63 (m, 2H, CH₂), 5.14-5.23 (m, 1H, CH), 7.09-7.16 (m, 2H, aryl-H), 7.25-7.31 (m, 2H, aryl-H), 7.46-7.48 (m, 1H, aryl-H), 7.55-7.62 (m, 2H, aryl-H), 7.81 (d, 1H, aryl-H), 7.89 (d, 1H, aryl-H), 8.16 (s, 1H, HCOOH), 8.62 (d, 1H, triazole-H).

### Example 43

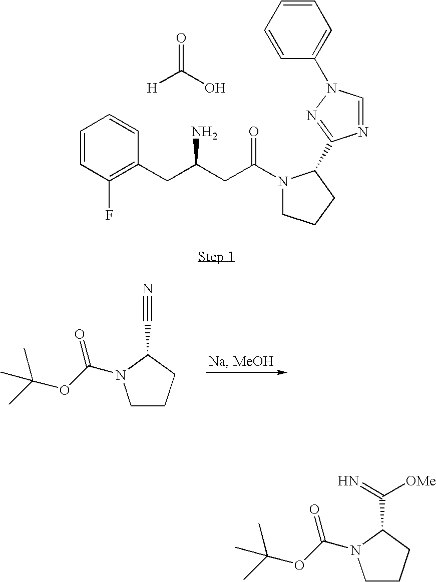

(S)-2-Methoxycarbonimidoyl-pyrrolidine-1-carboxylic acid tert-butyl ester

34 mg (1.49 mmol, 1.10 eq) of sodium is dissolved in 10 mL of methanol. Afterwards 265 mg (1.35 mmol, 1.00 eq) of (S)-2-cyano-pyrrolidine-1-carboxylic acid tert-butyl ester is added and the resulting solution is stirred for 14 h at room temperature. Then freshly prepared Dowex-resin is added to the reaction until an acidic pH is reached. The resin is filtered off and the filtrate is evaporated. The residue is taken up in diethyl ether, the mixture is filtered, and the filtrate is evaporated to yield the title compound which is used in the next step without further purification.

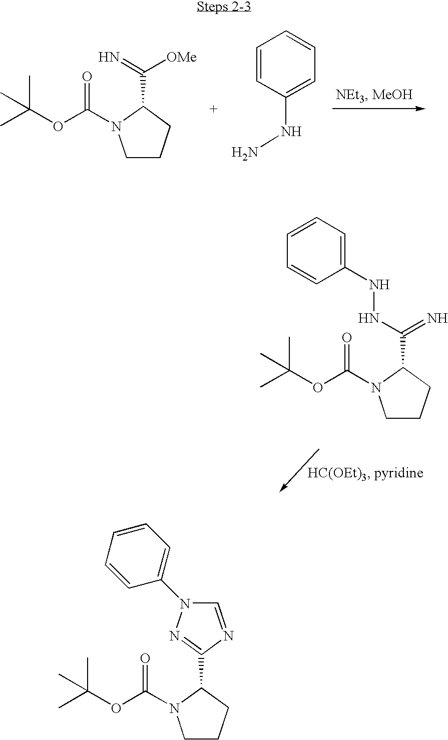

### (S)-2-(1-Phenyl-1H-[1,2,4]triazol-3-yl)-pyrrolidine-1-carboxylic acid tert-butyl ester

310 mg (1.36 mmol, 1.00 eq) of (S)-2-methoxycarbonimidoyl-pyrrolidine-1-carboxylic acid tert-butyl ester (Step 1) is dissolved in 5 mL of methanol and 135 μL (1.36 mmol, 1.00 eq) of phenylhydrazine and 522 μL (4.07 mmol, 3.00 eq) of triethylamine is added. The resulting solution is stirred for 3 days at room temperature and the solvent is evaporated. The residue is taken up in 5 mL of triethylorthoformate, treated with 250 μL (62.5 mmol, 46.0 eq) pyridine and stirred for 10 h at room temperature. After heating the reaction for 2 h to 120° C. the solvent is evaporated and the residue is taken up in dichloromethane. The organic solution is washed with 10% aqueous citric acid solution, saturated sodium bicarbonate solution and brine, dried with sodium sulphate and evaporated under reduced pressure. The residue is purified by column chromatography on silica gel (cyclo-hexane:ethyl acetate 50:50) to achieve the pure title compound.

LC/MS (I): rt 3.92; m/z 259, 315 [M+H]⁺, 378 [M+Na⁺ MeCN]⁺.

¹H-NMR (400 MHz, DMSO-d₆) δ=1.13-1.30 (d, 9H, CH₃), 1.84-2.08 (m, 4H, 2×CH₂), 3.24-3.50 (m, 4H, 2×CH₂), 4.83-4.92 (m, 1H, CH), 7.36-7.39 (m, 1H, aryl-H), 7.50-7.55 (m, 2H, aryl-H), 7.79-7.81 (m, 2H, aryl-H), 9.14-9.17 (d, 1H, triazole-H).

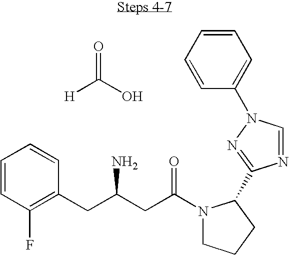

### (R)-3-Amino-4-(2-fluoro-phenyl)-1-((S)-2-{1-[(E)-((Z)-1-propenyl)-buta-1,3-dienyl]-1H-[1,2,4]triazol-3-yl}-pyrrolidin-1-yl)-butan-1-one formate salt

The title compound can be prepared by employing the deprotection, peptide coupling and deprotection sequence described for example 12, steps 4-6 with from step 2.

LC/MS (IV): rt 4.52; m/z 394 [M+H]⁺.

¹H-NMR (400 MHz, DMSO-d₆) δ=1.89-2.31 (m, 5H, CH, 2×CH₂), 2.65-2.88 (m, 2H, CH₂), 3.39-3.63 (m, 4H, 2×CH₂), 5.06-5.15 (m, 1H, CH), 7.07-7.31 (m, 5H, aryl-H), 7.41-7.56 (m, 2H, aryl-H), 7.74-7.83 (m, 2H, aryl-H), 8.19 (s, 1H, HCOOH), 9.16 (d, 1H, triazole-H).

### Example 44

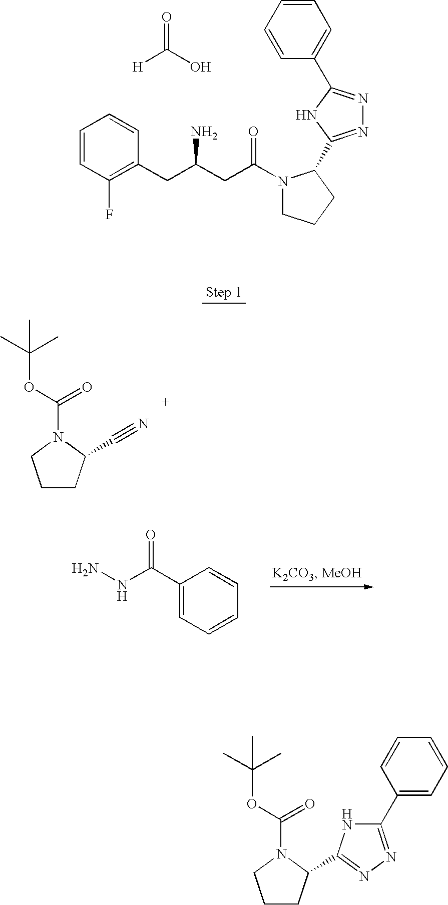

(S)-2-(5-Phenyl-4H-[1,2,4]triazol-3-yl)-pyrrolidine-1-carboxylic acid tert-butyl ester

111 mg (566 μmol, 1.00 eq) of (S)-2-cyano-pyrrolidine-1-carboxylic acid tert-butyl ester, 77 mg (566 μmol, 1.00 eq) of benzoic acid hydrazide and 117 mg (848 μmol, 1.50 eq) of potassium carbonate are heated to reflux in 4 mL of methanol for 18 h. Afterwards the solvent is evaporated and the product mixture is separated by column chromatography on silica gel (cyclo-hexane:ethyl acetate 30:70) thus yielding the title compound.

LC/MS (I): 15-95 gradient rt 2.25; m/z 315 [M+H]⁺, 356 [M+MeCN]⁺, 378 [M+Na⁺ MeCN]⁺.

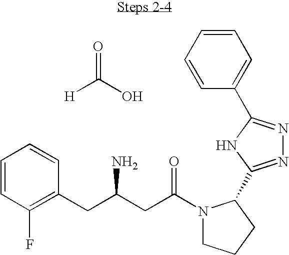

### (R)-3-Amino-4-(2-fluoro-phenyl)-1-((S)-2-{5-[(E)-((Z)-1-propenyl)-buta-1,3-dienyl]-4H-[1,2,4]triazol-3-yl}-pyrrolidin-1-yl)-butan-1-one formate salt

The title compound can be prepared by employing the deprotection, peptide coupling and deprotection sequence described for example 12, steps 4-6 with (S)-2-(5-phenyl-4H-[1,2,4]triazol-3-yl)-pyrrolidine-1-carboxylic acid tert-butyl ester from step 1.

LC/MS (III): rt 6.24; m/z 394 [M+H]⁺, 416 [M+Na]⁺.

### Example 45

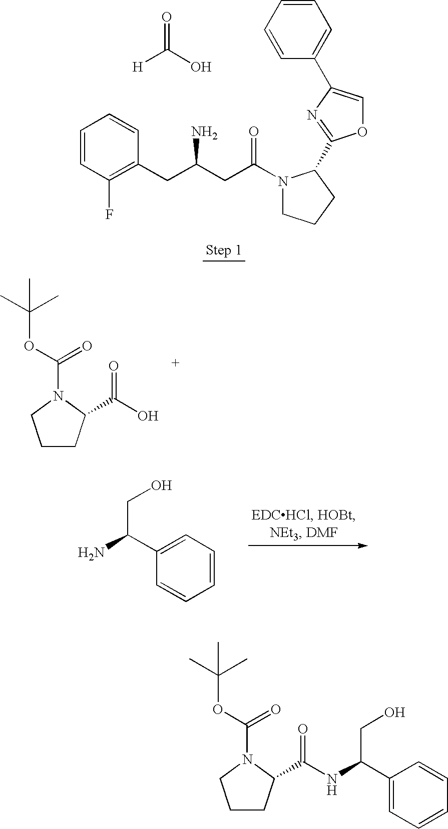

### (S)-2-((R)-2-Hydroxy-1-phenyl-ethylcarbamoyl)-pyrrolidine-1-carboxylic acid tert-butyl ester

369 mg (2.73 mmol, 1.50 eq) of 1-hydroxybenzotriazole, 910 μL (6.56 mmol, 3.60 eq) triethylamine and 524 mg (2.73 mmol, 1.50 eq) of 1-ethyl-3-(3-dimethylaminopropyl)carbodiimide hydrochloride are added to a stirred solution of 392 mg (1.82 mmol, 1.00 eq) of (S)-pyrrolidine-1,2-dicarboxylic acid 1-tert-butyl ester in 5 mL of N,N-dimethylformamide. The reaction mixture is stirred for 10 min, after which a solution of 250 mg (1.82 mmol, 1.00 eq) of (R)-2-amino-2-phenyl-ethanol in 5 mL of N,N-dimethylformamide is added at 0° C. The resulting solution is stirred for 12 h, then poured into brine and diluted with water. The aqueous mixture is extracted 3 times with ethyl acetate and the combined organic layers are washed with 5% citric acid solution, saturated sodium bicarbonate solution, brine and are dried with sodium sulphate. The solvent is evaporated and the residue is subjected to flash chromatography (silica gel, eluent: cyclo-hexane:ethyl acetate 30:70) to yield the pure title compound.

LC/MS (I): rt 3.15; m/z 335 [M+H]⁺, 372 [M+Na]⁺, 398 [M+Na⁺ MeCN]⁺

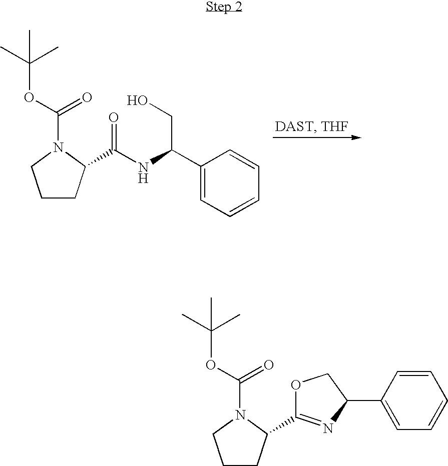

### (S)-2-((R)-4-Phenyl-4,5-dihydro-oxazol-2-yl)-pyrrolidine-1-carboxylic acid tert-butyl ester

50 mg (0.15 mmol, 1.00 eq) of (S)-2-((R)-2-hydroxy-1-phenyl-ethylcarbamoyl)pyrrolidine-1-carboxylic acid tert-butyl ester (Step 1) are dissolved in 1 mL of tetrahydrofuran and a solution of 39 mg (0.16 mmol, 1.10 eq) of N,N-diethylaminosulphor trifluoride in 500 μL tetrahydrofuran is added dropwise. The resulting mixture is stirred for 90 min at 70° C., the solvent is evaporated and the crude product is subjected to column chromatography on silica gel (cyclo-hexane:ethyl acetate 70:30) to yield the title compound.

LC/MS (I): rt 3.76; m/z 317 [M+H]⁺, 380 [M+Na⁺ MeCN]⁺

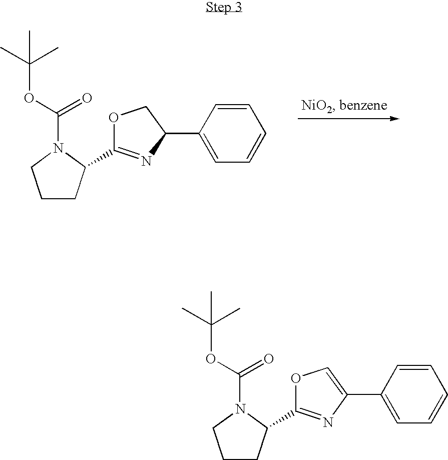

### (S)-2-(4-Phenyl-oxazol-2-yl)-pyrrolidine-1-carboxylic acid tert-butyl ester

30 mg (0.09 mmol, 1.00 eq) of (S)-2-((R)-4-phenyl-4,5-dihydro-oxazol-2-yl)-pyrrolidine-1-carboxylic acid tert-butyl ester (Step 2) is dissolved in 1 mL of benzene and 21 mg (0.19 mmol, 2.00 eq) of nickel peroxide hydrate is added. Then, the mixture is heated for 1 h at 150° C. in a microwave reactor, filtered, and the filtrate is evaporated. The residue is purified by column chromatography on silica gel (cyclo-hexane:ethyl acetate 90:10) to yield the title compound.

LC/MS (I): rt 4.48; m/z 259, 315 [M+H]⁺, 378 [M+Na⁺ MeCN]⁺

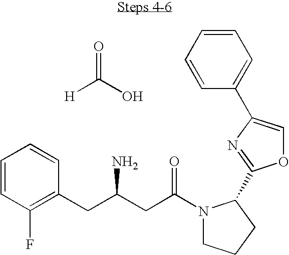

### (R)-3-Amino-4-(2-fluoro-phenyl)-1-((S)-2-{4-[(E)-((Z)-1-propenyl)-buta-1,3-dienyl]-oxazol-2-yl}-pyrrolidin-1-yl)-butan-1-one formate salt

The title compound can be prepared by employing the deprotection, peptide coupling and deprotection sequence described for example 12, steps 4-6 with (S)-2-(4-phenyloxazol-2-yl)-pyrrolidine-1-carboxylic acid tert-butyl ester from step 3.

LC/MS (IV): rt 5.21; m/z 394 [M+H]⁺, 416 [M+Na]⁺.

¹H-NMR (400 MHz, DMSO-d₆) δ=1.90-2.35 (m, 5H, CH, 2×CH₂), 2.65-2.82 (m, 2H, CH₂), 3.38-3.64 (m, 4H, 2×CH₂), 5.08-5.19 (m, 1H, CH), 7.09-7.18 (m, 2H, aryl-H), 7.24-7.46 (m, 5H, aryl-H), 7.69-7.77 (m, 2H, aryl-H), 8.22 (s, 1H, HCOOH), 8.52 (d, 1H, oxazole-H).

### Example 46

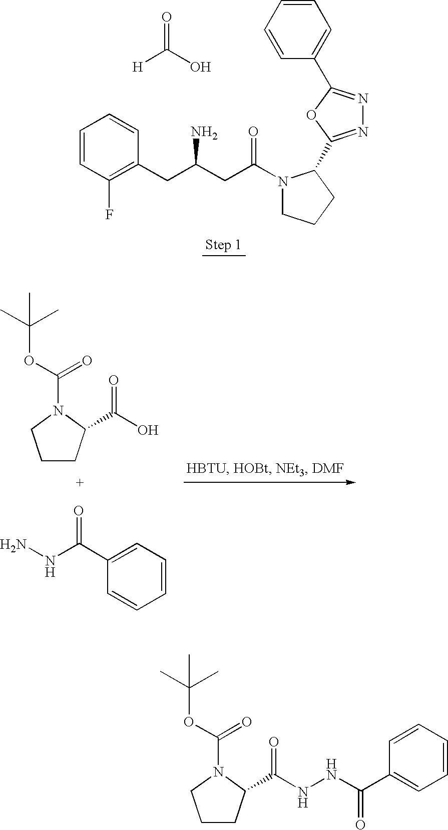

### (S)-2-(N′-Benzoyl-hydrazinocarbonyl)-pyrrolidine-1-carboxylic acid tert-butyl ester

596 mg (4.41 mmol, 1.20 eq) of 1-hydroxybenzotriazole, 1.13 mL (818 mg, 8.08 mmol, 2.20 eq) triethylamine and 1.67 g (4.41 mmol, 1.20 eq) of O-(benzotriazol-1-yl)-N,N,N′,N′-tetramethyluronium hexafluorophosphate are sequentially added to a stirred solution of 500 mg (3.67 mmol, 1.00 eq) of benzoic acid hydrazide and 790 mg (3.67 mmol, 1.00 eq) of (S)-pyrrolidine-1,2-dicarboxylic acid 1-tert-butyl ester in 15 mL of N,N-dimethylformamide. The resulting solution is stirred for 12 h at room temperature, then poured into brine and diluted with water. The aqueous mixture is extracted 3 times with ethyl acetate and the combined organic layers are washed with 5% citric acid solution, saturated sodium bicarbonate solution, brine and are dried with sodium sulphate. The solvent is evaporated and the residue is subjected to flash chromatography (silica gel, eluent: cyclo-hexane:ethyl acetate=1:1) to yield the title compound.

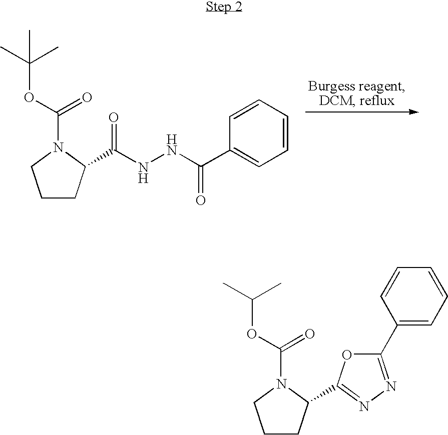

### (S)-2-(5-Phenyl-[1,3,4]oxadiazol-2-yl)-pyrrolidine-1-carboxylic acid tert-butyl ester

50 mg of (S)-2-(N′-benzoyl-hydrazinocarbonyl)-pyrrolidine-1-carboxylic acid tert-butyl ester (Step 1) is dissolved in 2 mL of dichloromethane and 54 mg (0.22 mmol, 1.50 eq) of Burgess reagent is added. The resulting solution is heated to reflux for 3 h. After cooling to room temperature the reaction mixture is washed saturated sodium bicarbonate solution, water and brine, dried with sodium sulphate, and the solvent is evaporated. The crude product is purified by column chromatography on silica gel (cyclo-hexane:ethyl acetate 70:30) to yield the title compound.

LC/MS (IV): rt 2.52; m/z 260, 301, 316 [M+H]⁺, 379 [M+Na⁺ MeCN]⁺.

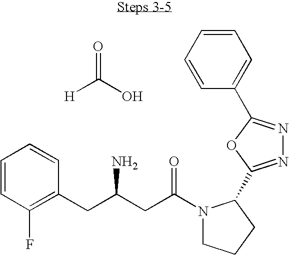

### (R)-3-Amino-4-(2-fluoro-Phenyl)-1-((S)-2-{5-[(E)-((Z)-1-propenyl)-buta-1,3-dienyl]-[1,3,4]oxadiazol-2-yl}-pyrrolidin-1-yl)-butan-1-one formate salt

The title compound can be prepared by employing the deprotection, peptide coupling and deprotection sequence described for example 12, steps 4-6 with (S)-2-(5-phenyl-[1,3,4]oxadiazol-2-yl)-pyrrolidine-1-carboxylic acid tert-butyl ester from step 2.

LC/MS (I): rt 2.85; m/z 395 [M+H]⁺, 417 [M+Na]⁺.

### Example 47

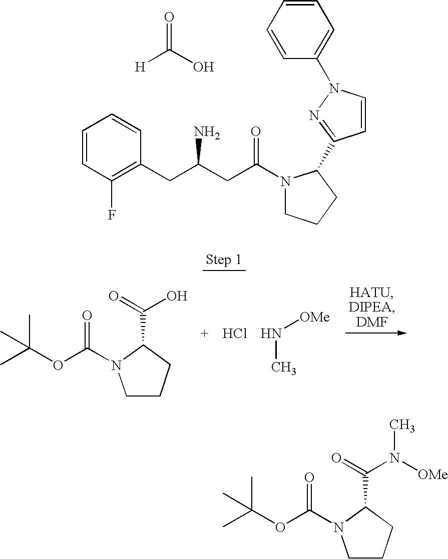

### (S)-2-(Methoxy-methyl-carbamoyl)-pyrrolidine-1-carboxylic acid tert-butyl ester

500 mg (2.32 mmol, 1.00 eq) of (S)-pyrrolidine-1,2-dicarboxylic acid 1-tert-butyl ester, 498 mg (5.11 mmol, 2.20 eq) N,O-dimethylhydroxylamine hydrochloride and 1.25 mL (6.97 mmol, 3.00 eq) of diisopropylethylamine are sequentially dissolved in 10 mL of N,N-dimethylformamide. Then 1.32 g (3.48 mmol, 1.50 eq) of O-(7-azabenzotriazol-1-yl)-N,N,N′,N′-tetramethyluronium hexafluorophosphate are added and the reaction mixture is stirred for 18 h at room temperature. Then, the solution is poured into brine and diluted with water. The aqueous mixture is extracted 3 times with ethyl acetate and the combined organic layers are washed with 5% citric acid solution, saturated sodium bicarbonate solution, brine and are dried with sodium sulphate. The solvent is evaporated and the residue is subjected to flash chromatography (silica gel, eluent: cyclo-hexane:ethyl acetate=50:50) to yield the title compound.

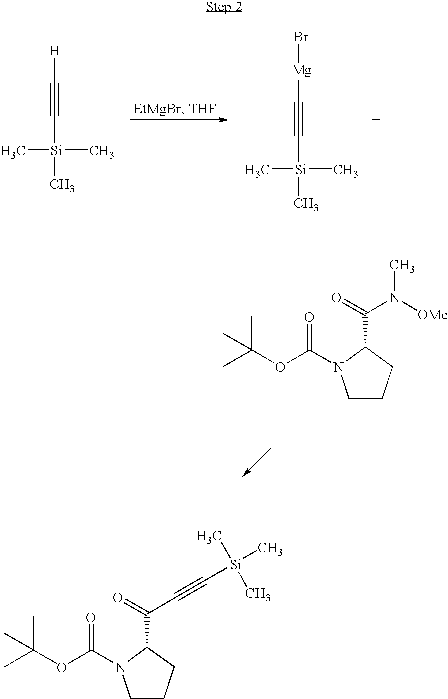

### (S)-2-(3-Trimethylsilanyl-propynoyl)-pyrrolidine-1-carboxylic acid tert-butyl ester

To a solution of 148 mg (1.51 mmol, 1.95 eq) of trimethylsilylacetylene in 500 μL tetrahydrofuran 1.08 mL (1.08 mmol, 1.40 eq) of a 1.0 M ethylmagnesium bromide solution in tetrahydrofuran is added dropwise. The resulting mixture is stirred for 1 h at room temperature, is then cooled to 0° C. and a solution of 278 mg (1.08 mmol, 1.00 eq) of (S)-2-(methoxy-methyl-carbamoyl)-pyrrolidine-1-carboxylic acid tert-butyl ester in 2.5 mL of tetrahydrofuran is added dropwise. After complete addition the reaction mixture is allowed to warm to room temperature and stirring is continued for 14 h. Then saturated ammonium chloride solution is added, and the mixture is extracted 3 times with ethyl acetate. The combined organic layers are dried with sodium sulphate, evaporated and the crude product is purified by column chromatography on silica gel (cyclo-hexane:ethyl acetate 80:20) affording the title compound.

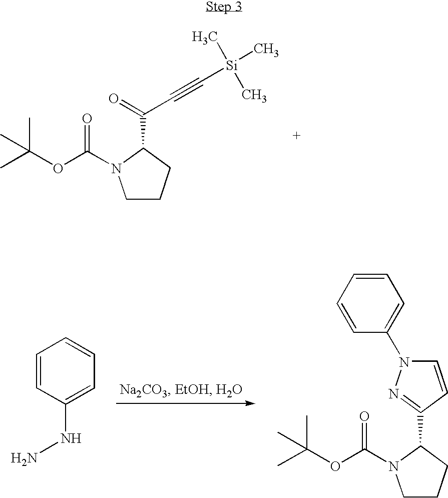

### (S)-2-(1-Phenyl-1H-pyrazol-3-yl)-pyrrolidine-1-carboxylic acid tert-butyl ester

50 mg (0.17 mmol, 1.00 eq) of (S)-2-(3-trimethylsilanyl-propynoyl)-pyrrolidine-1-carboxylic acid tert-butyl ester (Step 2) and 24 mg (0.22 mmol, 1.30 eq) of phenylhydrazine are dissolved in 2 mL of ethanol. The reaction mixture is heated to reflux and solution of 67 mg (0.23 mmol, 1.80 eq) sodium carbonate in 0.3 mL of water is added. The solution is refluxed overnight, the solvent is evaporated and the residue taken up in dichloromethane. The organic layer is washed with water, 10% aqueous citric acid solution and brine, dried with sodium sulphate and evaporated. The residue is subjected to column chromatography on silcia gel (cyclo-hexane:ethyl acetate 80:20) to afford the title compound.

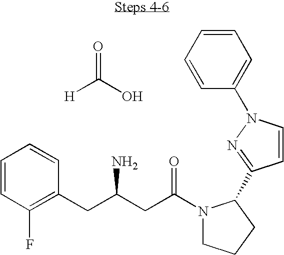

### (R)-3-Amino-4-(2-fluoro-phenyl)-1-((S)-2-{1-[(E)-((Z)-1-propenyl)-buta-1,3-dienyl]-1H-pyrazol-3-yl}-pyrrolidin-1-yl)-butan-1-one formate salt

The title compound can be prepared by employing the deprotection, peptide coupling and deprotection sequence described for example 12, steps 4-6 with (S)-2-(1-phenyl-1H-pyrazol-3-yl)-pyrrolidine-1-carboxylic acid tert-butyl ester from step 3.

LC/MS (IV): rt 4.79; m/z 393 [M+H]⁺, 415 [M+Na]⁺.

### Example 48

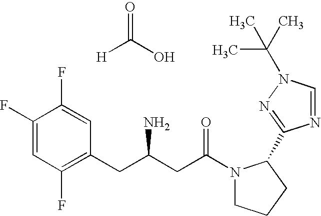

### 1H-[1,2,4]triazol-3-yl)-pyrrolidin-1-yl]-4-(2,4,5-trifluoro-phenyl)-butan-1-one

The title compound can be prepared according to example 43 by employing tert-butylhydrazine in step 2.

LC/MS (I): rt 2.37; m/z 411 (M+H)⁺.

¹H-NMR (400 MHz, DMSO-d₆) δ=1.45-1.49 (d, 9H, CH₃), 1.75-2.19 (m, 4H, CH₂), 2.50-2.87 (m, 4H, CH₂), 3.27-3.48 (m, 2H, CH, CH₂), 3.57-3.65 (m, 1H, CH₂), 5.02 (dd, 0.6H, CH, J=7.9 Hz, 2.3 Hz), 5.07 (dd, 0.4H, CH, J=5.6 Hz, 2.4 Hz), 7.39-7.57 (m, 2H, aryl-H), 8.13 (s, 1H, HCOOH), 8.33-8.47 (d, 1H, triazole-H).

### Example 49

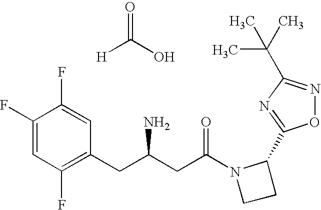

### (R)-3-Amino-1-[(S)-2-(3-tert-butyl-[1,2,4]oxadiazol-5-yl)-azetidin-1-yl]-4-(2,4,5-trifluorophenyl)-butan-1-one formate salt

The title compound can be prepared according to example 12 by employing (S)-azetidine-1,2-dicarboxylic acid 1-tert-butyl ester in step 2.

LC/MS (III): rt 8.44; m/z 397 [M+H]⁺, 438 [M+H+ MeCN]+

¹H-NMR (400 MHz, DMSO-d₆) δ=1.26-1.30 (m, 9H, 3×CH₃), 2.31-2.41 (m, 3H, CH, CH₂), 2.66-2.79 (m, 3H, CH, CH₂), 3.47-3.51 (m, 1H, CH), 3.87-4.20 (m, 2H, CH₂), 5.41-5.76 (m, 1H, CH), 7.42-7.51 (m, 2H, aryl-H), 8.14 (s, 1H, HCOOH).

### Example 50

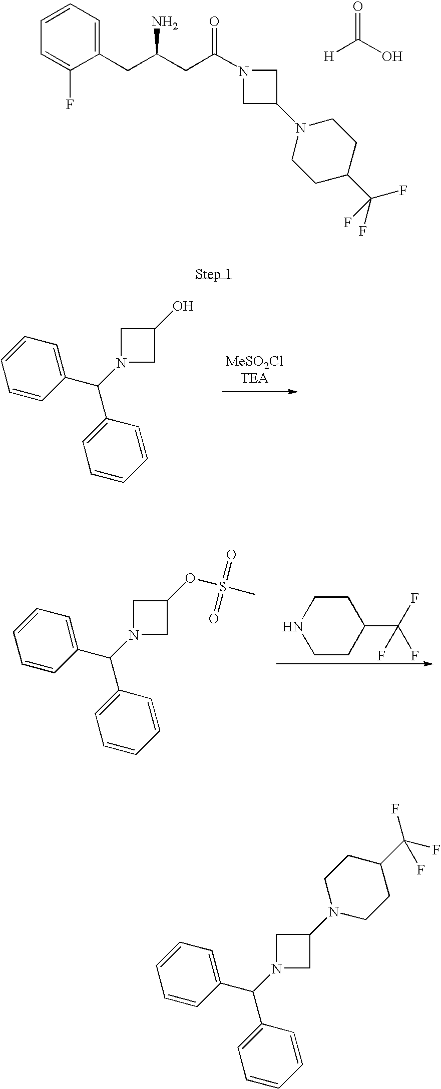

### 1-(1-Benzhydryl-azetidin-3-yl)-4-trifluoromethyl-piperidine

A solution of 200 mg (0.83 mmol) of 1-benzhydryl-azetidin-3-ol and 240 μL (1.68 mmol) triethylamine in 3.5 mL dichloromethane is cooled down to −40° C. 80 μL (1.01 mmol) of methanesulphonyl chloride are added and the reaction mixture is stirred by −40° C. for 30 minutes. After diluting with dichloromethane, the solution is washed with saturated aqueous sodium bicarbonate solution, dried over sodium sulphate and concentrated to dryness under vacuum yielding methanesulphonic acid 1-benzhydryl-azetidin-3-yl ester. A solution of 205 mg (4.2 mmol) of 4-trifluoromethyl-piperidine hydrochloride in 1 mL acetonitrile is stirred 1 h by room temperature in the presence of 750 mg (0.44 mmol) of (polystyrylmethyl)trimethylammoium bicarbonate resin. Then, after filtering off the resin, the solution of the free amine is poured onto the methanesulphonic acid 1-benzhydryl-azetidin-3-yl ester. The reaction mixture is heated at reflux for 7 days. After cooling and concentrating, the crude product is purified by flash chromatography (silica gel, cyclohexane to 5% of ethyl acetate in cyclohexane) affording the title compound.

LC/MS (II): 15-95 gradient rt 1.49; m/z 416 [M+H+MeCN]⁺.

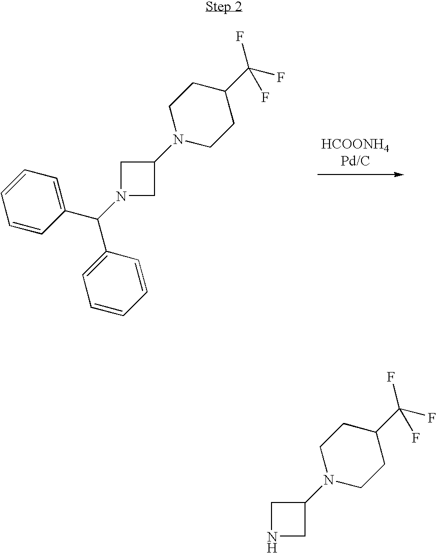

### 1-Azetidin-3-yl-4-trifluoromethyl-piperidine

A mixture of 50 mg (0.13 mmol) of 1-(1-benzhydryl-azetidin-3-yl)-4-trifluoromethyl-piperidine (Step 1), 25 mg (0.39 mmol) of ammonium formate and 16 mg of 10% palladium on charcoal in 2 mL of ethanol are stirred at reflux temperature for 30 minutes. After cooling down to room temperature, the reaction mixture is filtered through celite and concentrated to dryness. The resulting 1-azetidin-3-yl-4-trifluoromethyl-piperidine is used in the next step without further purification.

LC/MS (II): 15-95 gradient rt 0.35; m/z 209 [M+H]⁺.

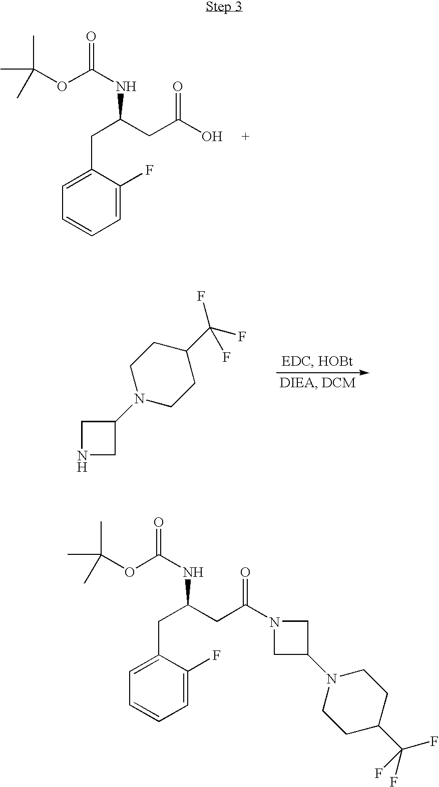

### (1R)-{1-(2-Fluoro-benzyl)-3-oxo-3-[3-(4-trifluoromethyl-piperidin-1-yl)-azetidin-1-yl]-propyl}carbamic acid tert-butyl ester

A mixture of 20 mg (0.07 mmol) of (3R)-tert-butoxycarbonylamino-4-[2-fluoro-phenyl]-butyric acid, 10 mg (0.07 mmol) of 1-hydroxybenzotriazole, 13 mg (0.07 mmol) of 1-ethyl-3-(3-dimethylaminopropyl)carbodiimide hydrochloride and 31,5 μL (0.18 mmol) of N,N-diisopropylethylamine in 0.5 mL of dichloromethane is stirred 30 minutes at room temperature. Then, 19 mg (0.09 mmol) 1-azetidin-3-yl-4-trifluoromethyl-piperidine (Step 2) in 0.5 mL of dichloromethane are added. The mixture is stirred overnight at room temperature. The solution is diluted with dichloromethane, washed sequentially with 1N hydrochloric acid, saturated aqueous sodium bicarbonate solution and brine, dried over sodium sulphate and concentrated to dryness under vacuum. The product is used in the next step without further purification.

LC/MS (II): 15-95 gradient rt 1.96; m/z 488 [M+H]⁺.

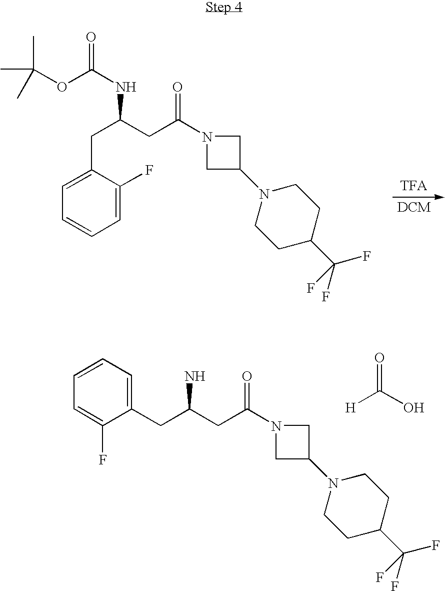

### (3R)-3-Amino-4-(2-fluoro-phenyl)-1-[3-(4-trifluoromethyl-piperidin-1-yl)-azetidin-1-yl]-butan-1-one

The product of step 3 is dissolved in 1 mL of a 30% solution of trifluoroacetic acid in dichloromethane and stirred 1.5 hours at room temperature. Then dichloromethane is added. The reaction mixture is concentrated under reduced pressure. This procedure is repeated 3 times. The crude product is purified by HPLC yielding pure (3R)-3-amino-4-(2-fluoro-phenyl)-1-[3-(4-trifluoromethyl-piperidin-1-yl)-azetidin-1-yl]-butan-1-one as formate salt.

LC/MS (IV): rt 3.84; m/z 388 [M+H]⁺.

¹H-NMR (DMSO-d₆): 1.40-1.60 (m, 2H), 1.77-1.87 (m, 3H), 2.17-2.32 (m, 3H), 2.79-2.88 (m, 3H), 3.10 (m, 2H), 3.51-3.64 (m, 3H), 3.81-3.86 (m, 2H), 4.02 (m, 1H), 7.12-7.17 (m, 2H), 7.26-7.33 (m, 2H), 8.13 (s, 0.4H).

### Example 51

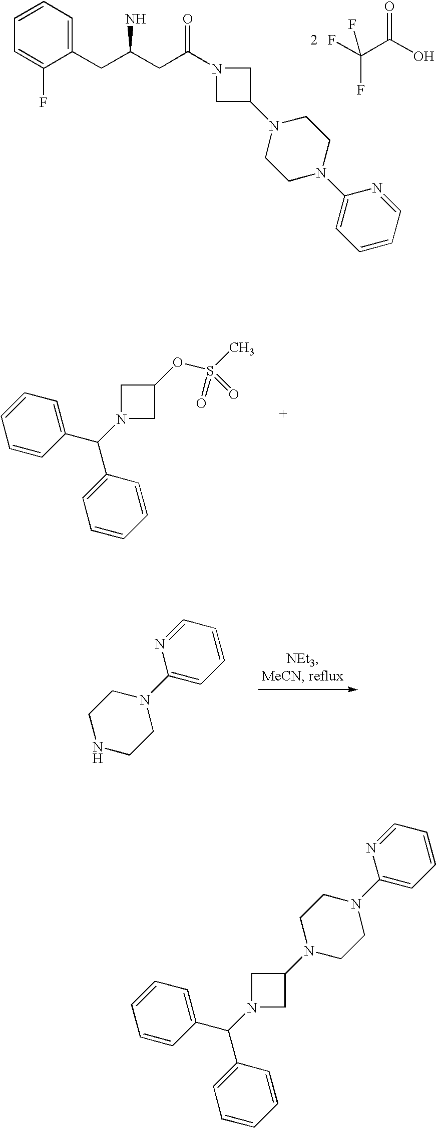

### 1-(1-Benzhydryl-azetidin-3-yl)-4-pyridin-2-yl-piperazine

615 μL (4.41 mmol) of triethylamine, 335.8 μL (2.21 mmol) of 1-(2-pyridyl)piperazine and 700 mg (2.21 mmol) of methanesuiphonic acid 1-benzhydryl-azetidin-3-yl ester (Example 50, Step 1) are heated to reflux in 10 mL of acetonitrile for 21 hours with stirring. After the mixture has cooled to room temperature, the solvent is evaporated. The residue is taken up in 30 mL of dicholoromethane, washed with 10 mL of water, dried with magnesium sulphat, filtered, and the solvent is evaporated. The residue is purified by column chromatography (silica gel, eluent: 1,2-dichloroethane-ethanol 5:1) and the resulting oil is crystallised in diethyl ether to give the title compound as a colorless powder.

¹H-NMR (CDCl₃): 2.40 (m, 4H), 2.92 (m, 2H), 2.99 (m, 1H), 3.42 (m, 2H), 3.54 (m, 4H), 4.40 (s, 1H), 6.60 (m, 1H), 6.61 (m, 1H), 7.15-7.40 (m, 10H), 7.44 (m, 1H), 8.07 (m, 1H).

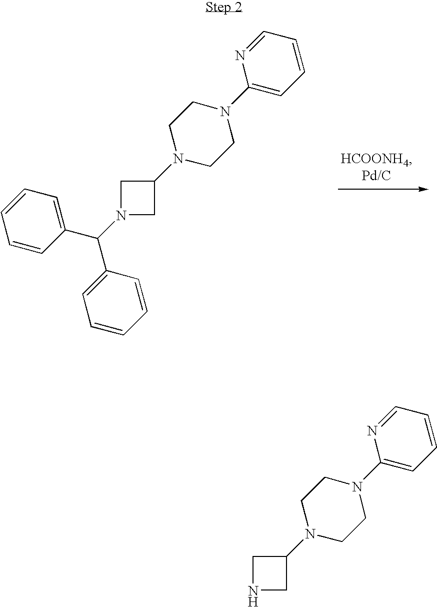

### 1-Azetidin-3-yl-4-pyridin-2-yl-piperazine

A mixture of 140 mg (0.36 mmol) of 1-(1-benzhydryl-azetidin-3-yl)-4-pyridin-2-yl-piperazine (Step 1), 69 mg (1.10 mmol) of ammonium formate and 42 mg of 10% palladium on charcoal in 11 mL of ethanol are stirred at reflux temperature overnight. After cooling to room temperature, the reaction mixture is filtered through celite and concentrated to dryness. The resulting 1-azetidin-3-yl-4-pyridin-2-yl-piperazine is used in the next step without further purification.

LC/MS (I): 5-70 gradient rt 0.35; m/z 219 [M+H]⁺.

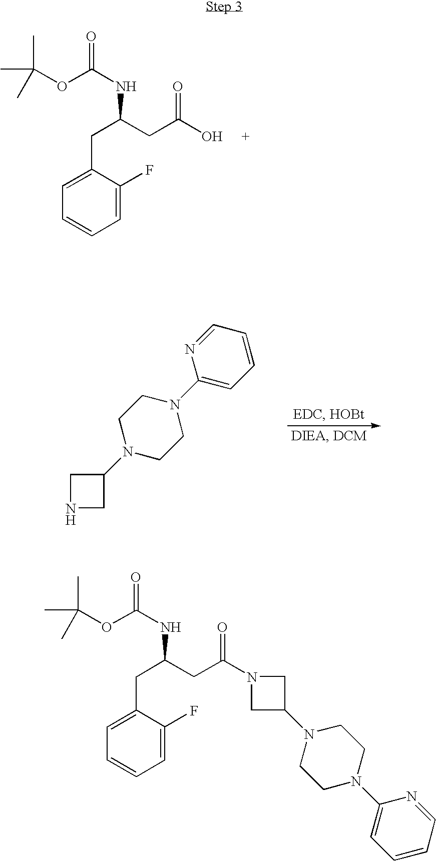

### (1R)-{1-(2-Fluoro-benzyl)-3-oxo-3-[3-(4-pyridin-2-yl-piperazin-1-yl)-azetidin-1-yl]-propyl}-carbamic acid tert-butyl ester

A mixture of 108 mg (0.36 mmol) of (3R)-tert-butoxycarbonylamino-4-[2-fluoro-phenyl]-butyric acid, 54 mg (0.40 mmol) of 1-hydroxybenzotriazole, 77 mg (0.40 mmol) of 1-ethyl-3-(3-dimethylaminopropyl)carbodiimide hydrochloride and 191 μL (1.09 mmol) of N,N-diisopropylethylamine in 7 mL of dichloromethane is stirred 30 minutes at room temperature. Then, 19 mg (0.09 mmol) 1-azetidin-3-yl-4-pyridin-2-yl-piperazine (Step 2) in 2 mL of dichloromethane are added. The mixture is stirred overnight at room temperature. The solution is diluted with dichloromethane, washed sequentially with saturated aqueous sodium bicarbonate solution and brine, dried over sodium sulphate and concentrated to dryness under vacuum. The product is used in the next step without further purification.

LC/MS (I): 5-70 gradient rt 2.53; m/z 498 [M+H]⁺.

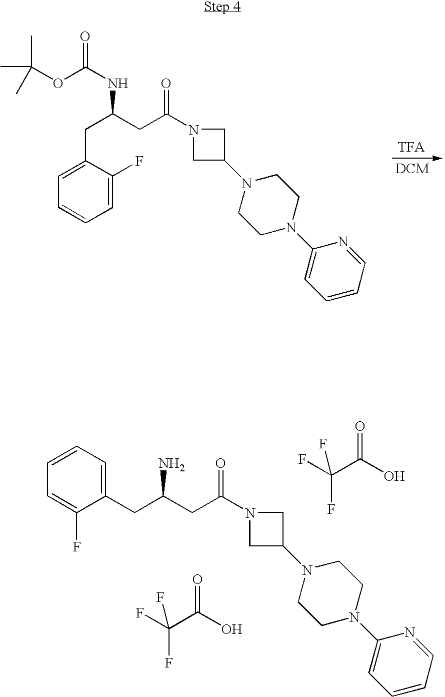

### (3R)-3-Amino-4-(2-fluoro-phenyl)-1-[3-(4-pyridin-2-yl-piperazin-1-yl)-azetidin-1-yl]-butan-1-one

The product of step 3 is dissolved in 2 mL of a 30% solution of trifluoroacetic acid in 15 dichloromethane and stirred 1 hour at room temperature. Then dichloromethane is added. The reaction mixture is concentrated under vacuum. This procedure is repeated 3 times. The crude product is purified by HPLC yielding pure (3R)-3-Amino-4-(2-fluorophenyl)-1-[3-(4-pyridin-2-yl-piperazin-1-yl)-azetidin-1-yl]-butan-1-one.

LC/MS (11): rt 4.75; m/z 398 [M+H]⁺.

¹H-NMR (DMSO-d₆): 2.27-2.38 (m, 2H), 2.86-3.09 (m, 6H), 3.60-3.80 (m, 6H), 3.96-4.06 (m, 2H), 4.24 (m, 2H), 6.75 (m, 1H), 7.00 (m, 1H), 7.15-7.19 (m, 2H), 7.29-7.34 (m, 2H7.62 (m, 1H), 7.98 (bs, 2H), 8.12 (m, 1H).

### Example 52

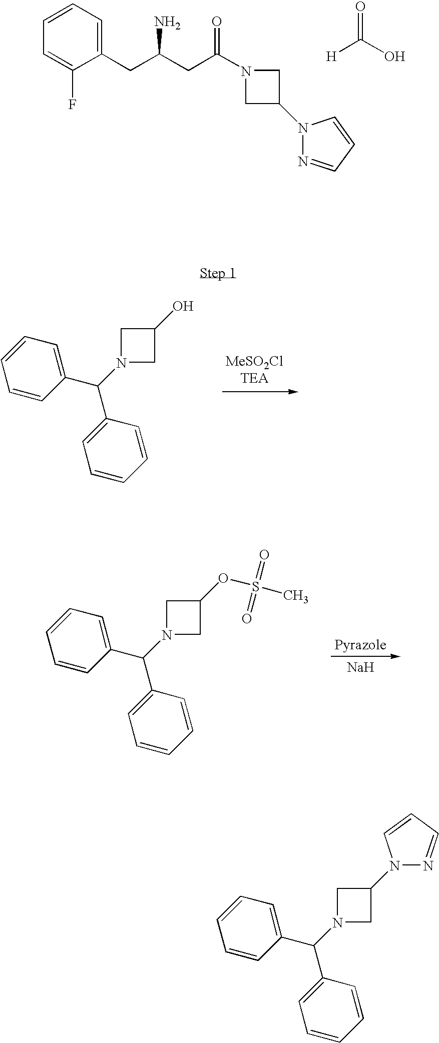

### 1-(1-Benzhydryl-azetidin-3-yl)-1H-pyrazole

A solution of 100 mg (0.42 mmol) of 1-benzhydryl-azetidin-3-ol and 117 μL (0.84 mmol) triethylamine in 0.5 mL dichloromethane is cooled to −40° C. 65 μL (0.50 mmol) of methanesulphonyl chloride are added and the reaction mixture is stirred at −40° C. for 1 hour. After diluting with dichloromethane, the solution is washed with saturated aqueous sodium bicarbonate solution, dried over sodium sulphate and concentrated to dryness under vacuum yielding methanesulphonic acid 1-benzhydryl-azetidin-3-yl ester. 30 mg (1.26 mmol) of sodium hydride (60% dispersion in mineral oil) are added to a solution of 57 mg (0.84 mmol) of pyrazole in 0.5 mL N,N-dimethylformamide. After stirring for 10 minutes at room temperature the mixture is poured onto a solution of the methanesulphonic acid 1-benzhydryl-azetidin-3-yl ester in 0.5 mL N,N-dimethylformamide. The reaction mixture is stirred overnight by 100° C. Then, after cooling to room temperature, ethyl acetate is added and the resulting mixture is washed with saturated aqueous sodium bicarbonate solution, brine, dried over sodium sulphate and concentrated under reduced pressure. The crude product is purified by flash chromatography (silica gel, cyclohexane to 25% of ethyl acetate in cyclohexane) affording the title compound.

LC/MS (I): 15-95 gradient rt 1.50; m/z 290 [M+H]⁺.

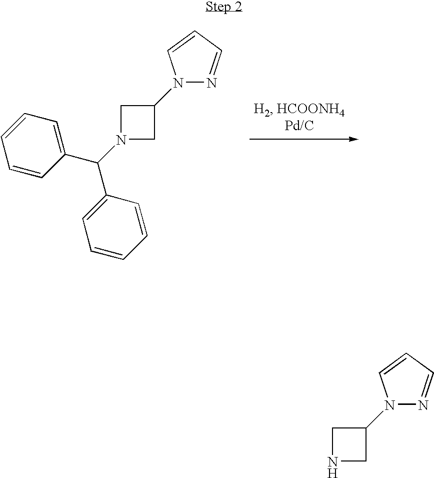

### 1-Azetidin-3-yl-1H-pyrazole

A mixture of 91 mg (0.31 mmol) of 1-(1-benzhydryl-azetidin-3-yl)-1H-pyrazole (Step 1), δ 25 mg (0.39 mmol) of ammonium formate and 30 mg of 10% palladium on charcoal in 2 mL of ethanol are stirred at reflux temperature for 30 minutes. Then, a further 30 mg portion of 10% palladium on charcoal are added and the reaction mixture is stirred 3 days at room temperature under a hydrogen atmosphere. The reaction mixture is filtered through celite and concentrated to dryness. The resulting 1-azetidin-3-yl-1H-pyrazole is used in the next step without further purification.

LC/MS (I): 15-95 gradient rt 0.35; m/z 124 [M+H]⁺.

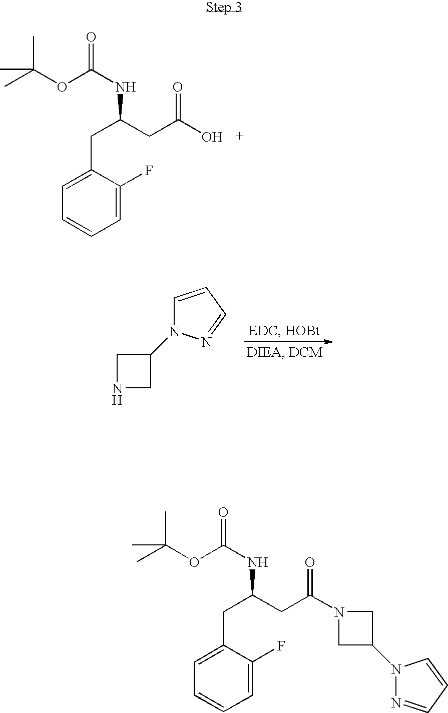

### (1R)-[1-(2-Fluoro-benzyl)-3-oxo-3-(3-pyrazol-1-yl-azetidin-1-yl)-propyl]-carbamic acid tert-butyl ester

A mixture of 184 mg (0.62 mmol) of (3R)-tert-butoxycarbonylamino-4-[2-fluoro-phenyl]-butyric acid, 95 mg (0.70 mmol) of 1-hydroxybenzotriazole, 133 mg (0.70 mmol) of 1-ethyl-3-(3-dimethylaminopropyl)carbodiimide hydrochloride and 325 μL (1.86 mmol) of N,N-diisopropylethylamine in 2 mL of dichloromethane is stirred 30 minutes at room temperature. Then, 76 mg (0.62 mmol) 1-azetidin-3-yl-1H-pyrazole (Step 2) in 0.5 mL of dichloromethane are added. The mixture is stirred overnight at room temperature. The solution is diluted with dichloromethane, washed sequentially with 1N hydrochloric acid, saturated aqueous sodium bicarbonate solution and brine, dried over sodium sulphate and concentrated to dryness under vacuum. The product is used in the next step without further purification.

LC/MS (I): 15-95 gradient rt 2.28; m/z 403 [M+H]⁺.

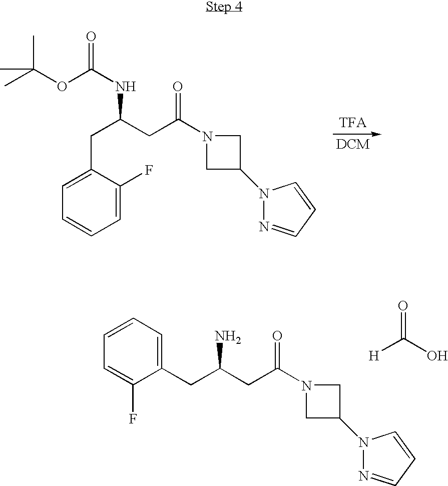

### (3R)-3-Amino-4-(2-fluoro-phenyl)-1-(3-pyrazol-1-yl-azetidin-1-yl)-butan-1-one

The product of step 3 is dissolved in 1 mL of a 30% solution of trifluoroacetic acid in dichloromethane and stirred 1.5 hours at room temperature. Then dichloromethane is added. The reaction mixture is concentrated under vacuum. This procedure is repeated 3 times. The crude product is purified by HPLC yielding pure (3R)-3-amino-4-(2-fluorophenyl)-1-(3-pyrazol-1-yl-azetidin-1-yl)-butan-1-one as the formate salt.

LC/MS (II): rt 5.02; m/z 303 [M+H]⁺.

¹H-NMR (DMSO-d₆): 2.17 (m, 2H), 2.73 (m, 2H), 3.38 (m, 1H), 4.07-4.12 (m, 1H), 4.22-4.39 (m, 2H), 4.43-4.55 (m, 1H), 5.51-5.24 (m, 1H), 6.27 (m, 1H), 7.09-7.14 (m, 2H), 7.24-7.32 (m, 2H), 7.52 (s, 1H), 7.83 (s, 1H), 8.18 (s, 0.7H).

### Example 53 and 54

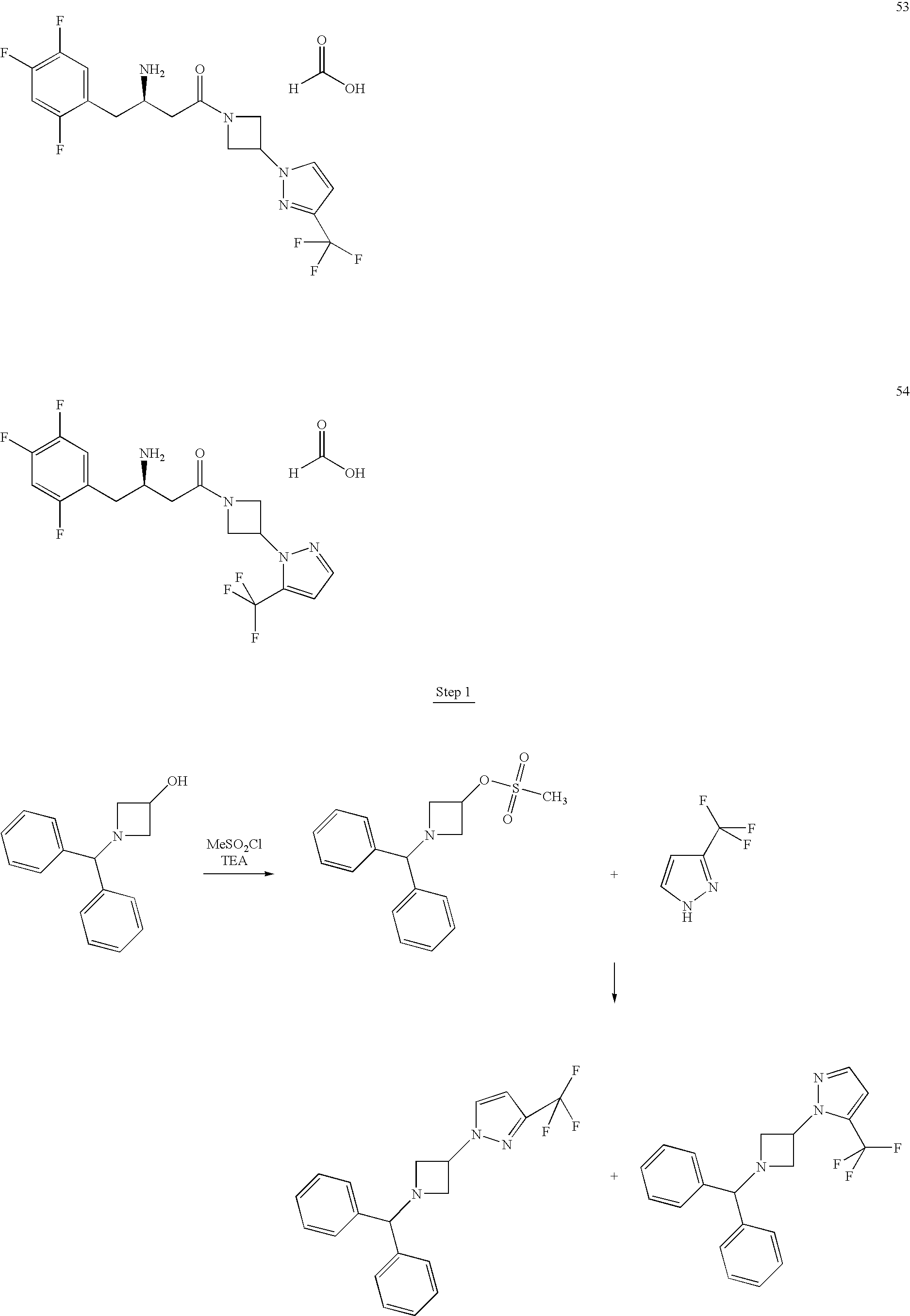

### 1-(1-Benzhydryl-azetidin-3-yl)-3-trifluoromethyl-1H-pyrazole and 1-(1-benzhydryl-azetidin-3yl)-5-trifluoromethyl-1H-pyrazole

A solution of 440 mg (1.85 mmol) of 1-benzhydryl-azetidin-3-ol and 535 μL (3.68 mmol) triethylamine in 7 mL dichloromethane is cooled to −40° C. 178 μL (2.2 mmol) of methanesulphonyl chloride are added and the reaction mixture is stirred at 40° C. for 30 minutes. After adding dichloromethane, the solution is washed with saturated aqueous sodium bicarbonate solution, dried over sodium sulphate and concentrated to dryness under vacuum yielding methanesulphonic acid 1-benzhydryl-azetidin-3-yl ester. 295 mg (7.3 mmol) of sodium hydride (60% dispersion in mineral oil) are added to a solution of 500 mg (3.67 mmol) of 3-trifluoromethyl-1H-pyrazole in 2 mL tetrahydrofuran. After stirring 10 minutes at room temperature, a solution of the methanesulphonic acid 1-benzhydryl-azetidin-3-yl ester in 3 mL tetrahydrofuran is added. The reaction mixture is stirred under microwave heating at 100° C. for 20 minutes. Then after cooling to room temperature, ethyl acetate is added and the resulting mixture is washed with saturated aqueous sodium bicarbonate solution, brine, dried over sodium sulphate and concentrated to dryness. The crude product is purified by flash chromatography (silica gel, cyclohexane to 25% of ethyl acetate in cyclohexane) affording the title compounds.

LC/MS (II): rt 3.21 and 3.32; m/z 358 [M+H]⁺.

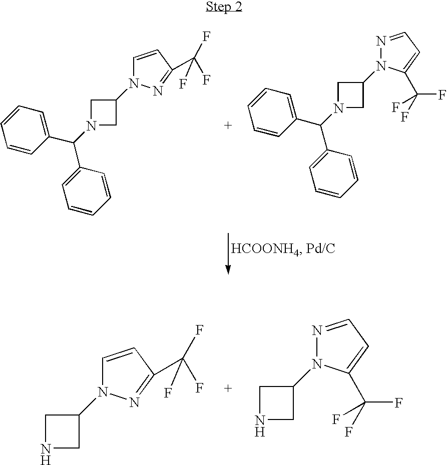

### 1-Azetidin-3-yl-3-trifluoromethyl-1H-pyrazole and 1-azetidin-3-yl-5-trifluoromethyl-1H-pyrazole

A mixture of 320 mg (0.87 mmol) 1-(1-benzhydryl-azetidin-3-yl)-3-trifluoromethyl-1H-pyrazole and 1-(1-benzhydryl-azetidin-3-yl)-5-trifluoromethyl-1H-pyrazole (Step 1), 164 mg (2.61 mmol) of ammonium formate and 110 mg of 10% palladium on charcoal in 5 mL of ethanol are stirred at reflux temperature for 30 minutes. After cooling to room temperature, the reaction mixture is filtered through celite and concentrated to dryness. The 1-azetidin-3-yl-3-trifluoromethyl-1H-pyrazole and 1-azetidin-3-yl-5-trifluoromethyl-1H-pyrazole title compounds are used in the next step without further purification.

LC/MS (I): rt 1.51 and 1.66; m/z 192 [M+H]⁺.

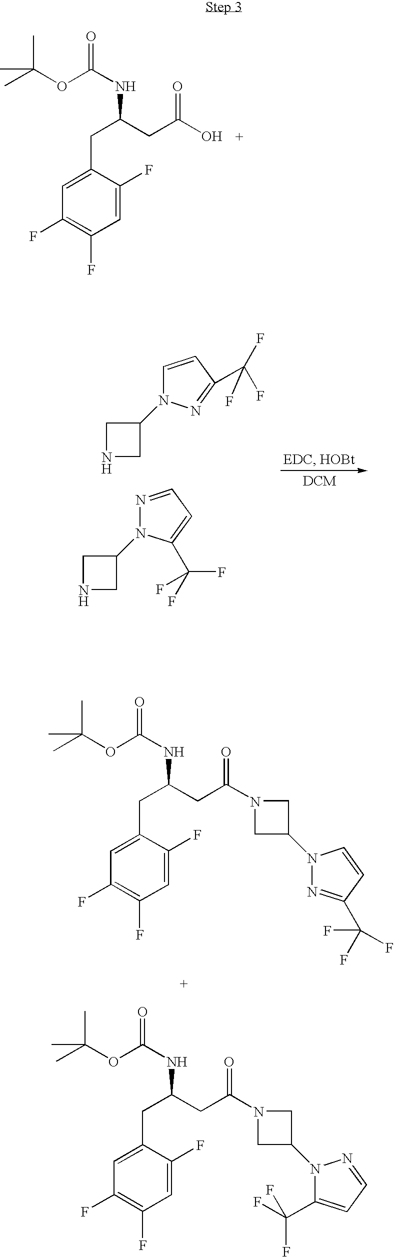

### (1R)-{3-Oxo-1-(2,4,5-trifluoro-benzyl)-3-[3-(3-trifluoromethyl-pyrazol-1-yl)-azetidin-1-yl]-propyl}carbamic acid tert-butyl ester and (1R)-[3-oxo-1-(2,4,5-trifluoro-benzyl)-3-[3-(5-trifluoromethyl-pyrazol-1-yl)-azetidin-1-yl]-propyl]-carbamic acid tert-butyl ester

A mixture of 289 mg (0.87 mmol) of (3R)-3-tert-butoxycarbonylamino-4-(2,4,5-trifluorophenyl)-butyric acid, 128 mg (0.95 mmol) of 1-hydroxybenzotriazole, 182 mg (0.95 mmol) of 1-ethyl-3-(3-dimethylaminopropyl)carbodiimide hydrochloride and 330 μL (1.88 mmol) of N,N-diisopropylethylamine in 6 mL of dichloromethane is stirred 30 minutes at 0° C. Then, 166 mg (0.87 mmol) of a mixture of 1-azetidin-3-yl-3-trifluoromethyl-1H-pyrazole and 1-azetidin-3-yl-5-trifluoromethyl-1H-pyrazole (Step 2) 2 mL of dichloromethane are added. The mixture is stirred overnight at room temperature. The solution is diluted with dichloromethane, washed sequentially with 1N hydrochloric acid, saturated aqueous sodium bicarbonate solution and brine, dried over sodium sulphate and concentrated to dryness under vacuum. The product is used in the next step without further purification.

LC/MS (II): 5-80 gradient: rt 5.48 and 5.58; m/z 507 [M+H]⁺.

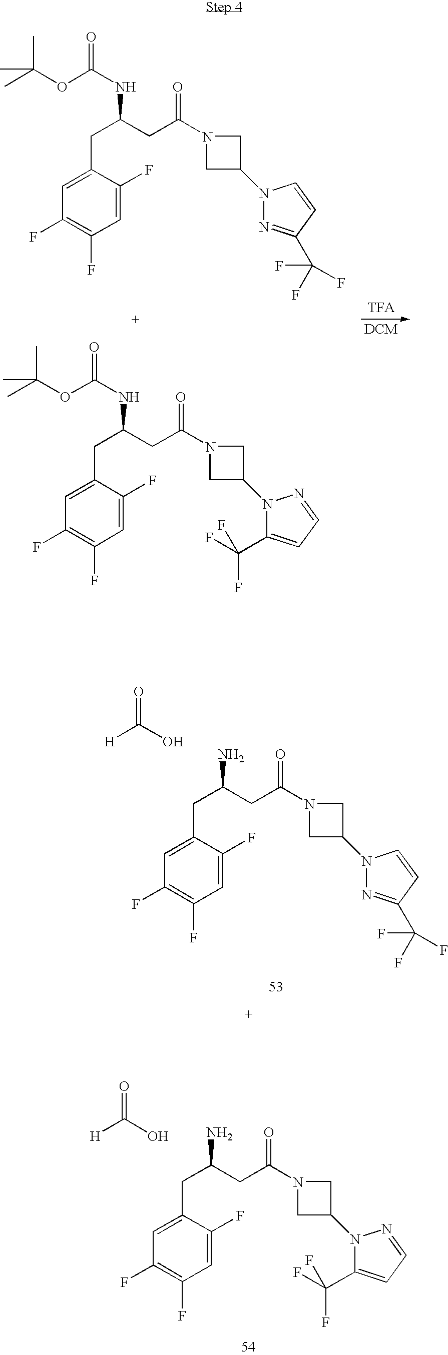

### (3R)-3-Amino-1-[3-(3-trifluoromethyl-pyrazol-1-yl)-azetidin-1-yl]-4-(2,4,5-trifluorophenyl)-butan-1-one (53) and (3R)-3-amino-1-[3-(5-trifluoromethyl-pyrazol-1-yl)azetidin-1-yl]-4-(2,4,5-trifluoro-phenyl)-butan-1-one (54)

The products of step 3 is dissolved in 3 mL of a 30% solution of trifluoroacetic acid in dichloromethane and stirred 45 minutes at 0° C. Then dichloromethane is added. The reaction mixture is concentrated under vacuum. This procedure is repeated 3 times. The crude product is purified by HPLC yielding both regioisomers (3R)-3-amino-1-[3-(3-trifluoromethyl-pyrazol-1-yl)-azetidin-1-yl]-4-(2,4,5-trifluoro-phenyl)-butan-1-one (53) and (3R)-3-amino-1-[3-(5-trifluoromethyl-pyrazol-1-yl)-azetidin-1-yl]-4-(2,4,5-trifluorophenyl)-butan-1-one (54) as formate salts.

53: LC/MS (IV): rt 4.37; m/z 407 [M+H]⁺.

¹H-NMR (DMSO-d₆): 2.32-2.38 (m, 2H), 2.85 (m, 2H), 3.61 (m, 1H), 4.25 (m, 1H), 4.34 (m, 1H), 4.45 (m, 1H), 4.59 (m, 1H), 5.35 (m, 1H), 7.00 (m, 1H), 7.47-7.57 (m, 2H), 7.84 (m, 1H), 8.14 (s, 0.5H).

54: LC/MS (IV): rt 4.64; m/z 407 [M+H]⁺.

¹H-NMR (DMSO-d₆): 2.32-2.38 (m, 2H), 2.87 (m, 2H), 3.66 (m, 1H), 4.08-4.15 (m, 1H), 4.30-4.44 (m, 2H), 4.54-4.61 (m, 1H), 5.35 (m, 1H), 6.81 (m, 1H), 7.48-7.57 (m, 2H), 8.14 (s, 1.2H).

### Examples 55 and 56

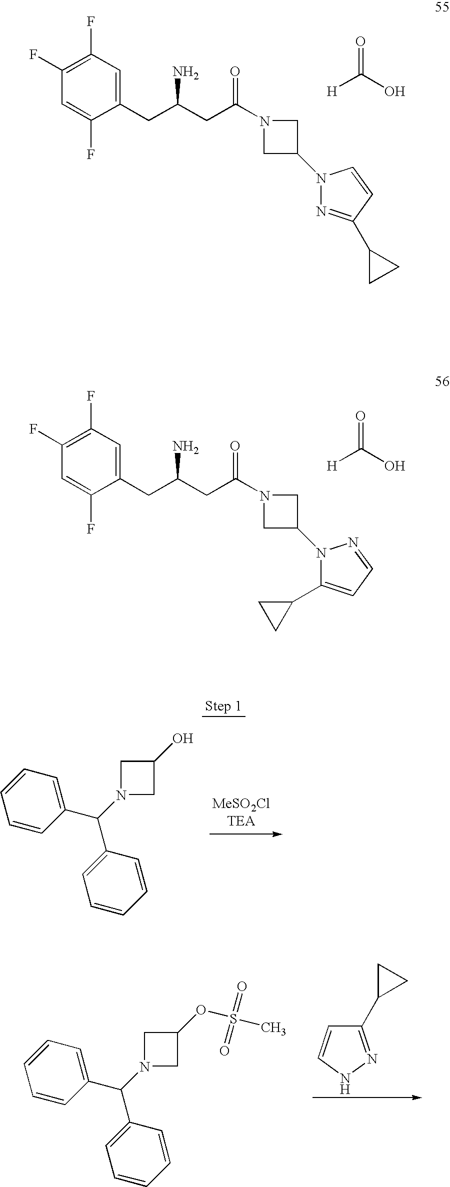

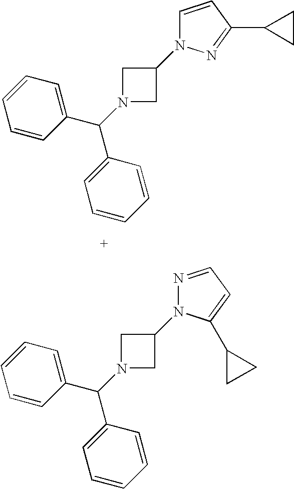

### 1-(1-Benzhydryl-azetidin-3-yl)-3-cyclopropyl-1H-pyrazole and 1-(1-benzhydryl-azetidin-3-yl)-5-cyclopropyl-1H-pyrazole

A solution of 100 mg (0.42 mmol) of 1-benzhydryl-azetidin-3-ol and 118 μL (0.84 mmol) triethylamine in 2 mL dichloromethane is cooled down to −40° C. 40 μL (0.51 mmol) of methanesulphonyl chloride are added and the reaction mixture is stirred at 40° C. for 30 minutes. After diluting with dichloromethane, the solution is washed with saturated aqueous sodium bicarbonate solution, dried over sodium sulphate and concentrated to dryness under vacuum to yield methanesulphonic acid 1-benzhydryl-azetidin-3-yl ester. 41 mg (1 mmol) of sodium hydride (60% dispersion in mineral oil) are added to a 15 solution of 91 mg (0.84 mmol) of 3-cyclopropyl-1H-pyrazole in 0.75 mL tetrahydrofuran. After stirring 10 minutes at room temperature, a solution of the methanesulphonic acid 1-benzhydryl-azetidin-3-yl ester in 0.75 mL tetrahydrofuran is added. The reaction mixture is stirred under microwave heating at 100° C. 20 minutes. Then after cooling to room temperature and evaporating the tetrahydrofuran, ethyl acetate is added and the resulting mixture is washed with saturated aqueous sodium bicarbonate solution, with brine, dried over sodium sulphate and concentrated to dryness. The products are used in the next step without further purification.

LC/MS (II): rt 2.79; m/z 330 [M+H]⁺.

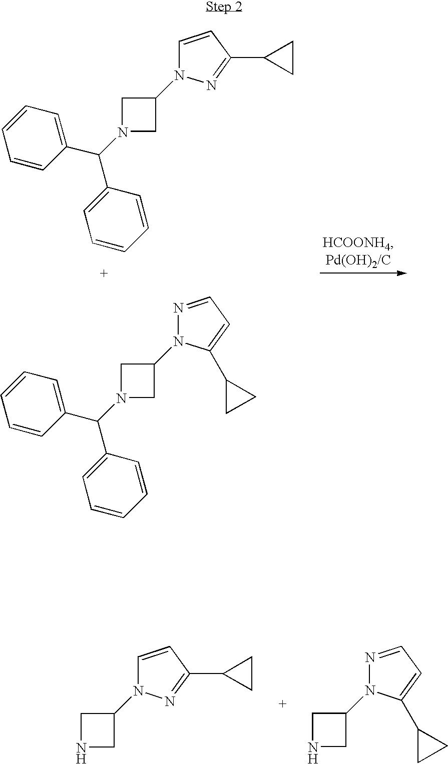

### 1-Azetidin-3-yl-3-cyclopropyl-1H-pyrazole and 1-azetidin-3-yl-5-cyclopropyl-1H-pyrazole

A mixture of 173 mg (0.53 mmol) 1-(1-benzhydryl-azetidin-3-yl)-3-cyclopropyl-1H-pyrazole and 1-(1-benzhydryl-azetidin-3-yl)-5-cyclopropyl-1H-pyrazole (Step 1), 103 mg (1.59 mmol) of ammonium formate and 50 mg of 10% palladium on charcoal in 5 mL of ethanol are stirred overnight at reflux temperature. After cooling down to room temperature, 50 mg of 10% palladium(II) hydroxide on charcoal are added and the reaction mixture is stirred under hydrogen atmosphere for 3 days. After filtering the catalysts through celite and removing the solvent under vacuum a mixture of 1-azetidin-3-yl-3-cyclopropyl-1H-pyrazole and 1-azetidin-3-yl-5-cyclopropyl-1H-pyrazole are obtained. They are used in the next step without further purification.

LC/MS (II): rt 1.39; m/z 164 [M+H]⁺.

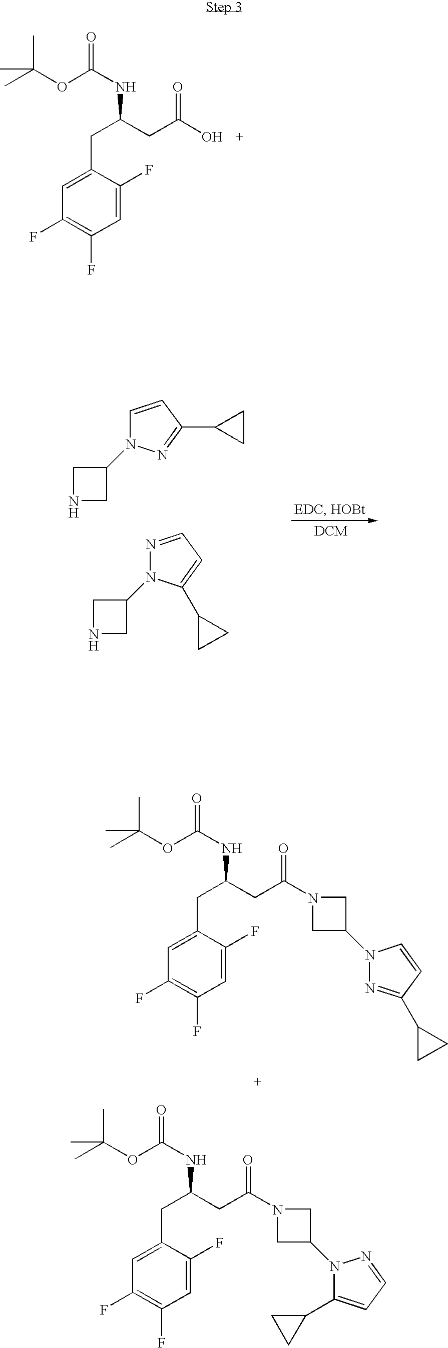

### [(R)-3-[3-(3-Cyclopropyl-pyrazol-1-yl)-azetidin-1-yl]-3-oxo-1-(2,4,5-trifluoro-benzyl)propyl]carbamic acid tert-butyl ester and [(R)-3-[3-(5-cyclopropyl-pyrazol-1-yl)-azetidin-1-yl]3-oxo-1-(2,4,5-trifluoro-benzyl)-propyl]-carbamic acid tert-butyl ester

A mixture of 193 mg (0.58 mmol) of (3R)-3-tert-butoxycarbonylamino-4-(2,4,5-trifluorophenyl)-butyric acid (Step 2), 78 mg (0.58 mmol) of 1-hydroxybenzotriazole, 111 mg (0.58 mmol) of 1-ethyl-3-(3-dimethylaminopropyl)carbodiimide hydrochloride and 222 μL (1.27 mmol) of diisopropylethylamine in 1 mL of dichloromethane is stirred 30 minutes at 0° C. Then, 86 mg (0.53 mmol) of a mixture of 1-azetidin-3-yl-3-cyclopropyl-1H-pyrazole and 1-azetidin-3-yl-5-cyclopropyl-1H-pyrazole in 0.5 mL of dichloromethane is added. The reaction mixture is stirred overnight at room temperature. The solution is diluted with dichloromethane, washed sequentially with 1N hydrochloric acid, saturated aqueous sodium bicarbonate solution and brine, dried over sodium sulphate and concentrated to dryness to yield the title compounds which are used in the next step without further purification.

LC/MS (11): rt 4.18; m/z 479 [M+H]⁺.

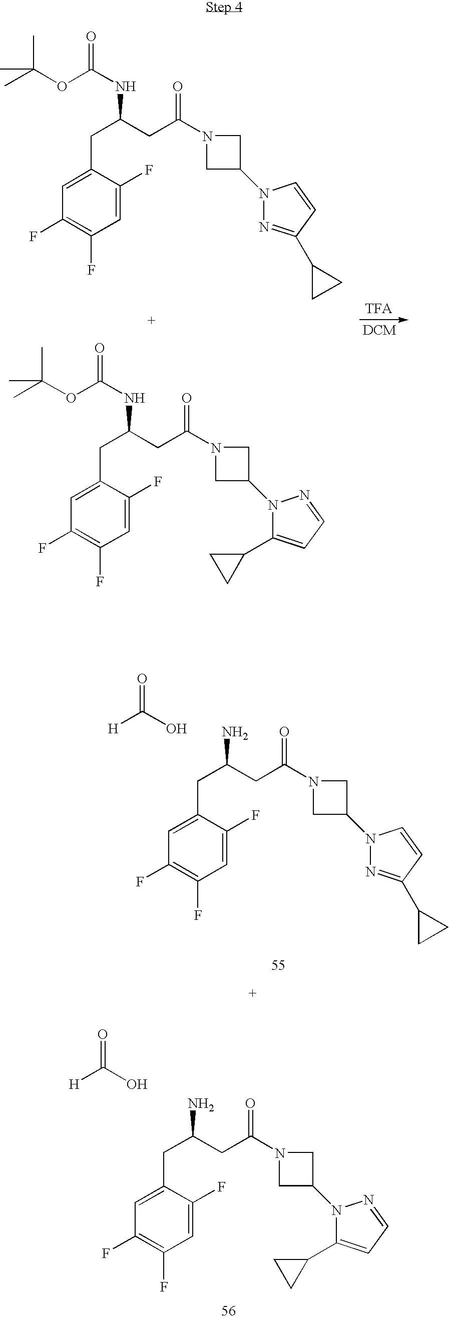

### (R)-3-Amino-1-[3-(3-cyclopropyl-pyrazol-1-yl)-azetidin-1-yl]-4-(2,4,5-trifluoro-phenyl)butan-1-one (55) and (R)-3-amino-1-[3-(5-cyclopropyl-pyrazol-1-yl)-azetidin-1-yl]-4-(2,4,5-trifluoro-phenyl)-butan-1-one (56)

The products of step 3 is dissolved in 1.4 mL of a 30% solution of trifluoroacetic acid in dichloromethane and stirred 45 minutes at 0° C. Then methanol is added and the reaction mixture is concentrated to dryness. The crude product is purified by HPLC yielding both regioisomers (R)-3-amino-1-[3-(3-cyclopropyl-pyrazol-1-yl)-azetidin-1-yl]-4-(2,4,5-trifluoro-phenyl)-butan-1-one (55) and (R)-3-amino-11-[3-(5-cyclopropyl-pyrazol-1-yl)-azetidin-1-yl]-4-(2,4,5-trifluoro-phenyl)-butan-1-one (56) as formate salts.

55: LC/MS (IV): rt 7.34; m/z 379 [M+H]⁺.

¹H-NMR (DMSQ-d₆): 0.58 (m, 2H), 0.92 (m, 2H), 1.85 (m, 1H), 2.31 (m, 2H), 2.79 (m, 2H), 3.50 (m, 1H), 4.16 (m, 1H), 4.32 (m, 1H), 4.42 (m, 1H), 4.54 (m, 1H), 5.42 (m, 1H), 5.94 (s, 1H), 7.41 (s, 1H), 7.51 (m, 2H), 8.17 (s, 1H).

56: LC/MS (IV): rt 7.19; m/z 379 [M+H]⁺.

¹H-NMR (DMSO-d₆): 0.61 (m, 2H), 0.94 (m, ZH), 1.87 (m, 1H), 2.30 (m, 2H), 2.80 (m, 2H), 3.53 (m, 1H), 4.05-4.57 (m, 4H), 5.14 (m, 1H), 5.95 (s, 1H), 7.41 (m, 2H), 7.69 (s, 1H), 8.16 (s, 1H).

Further examples from this series are exemplified below:

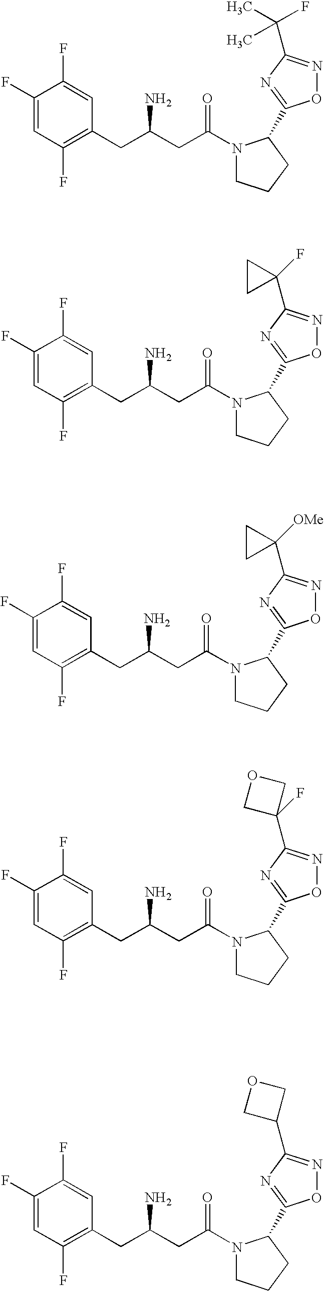

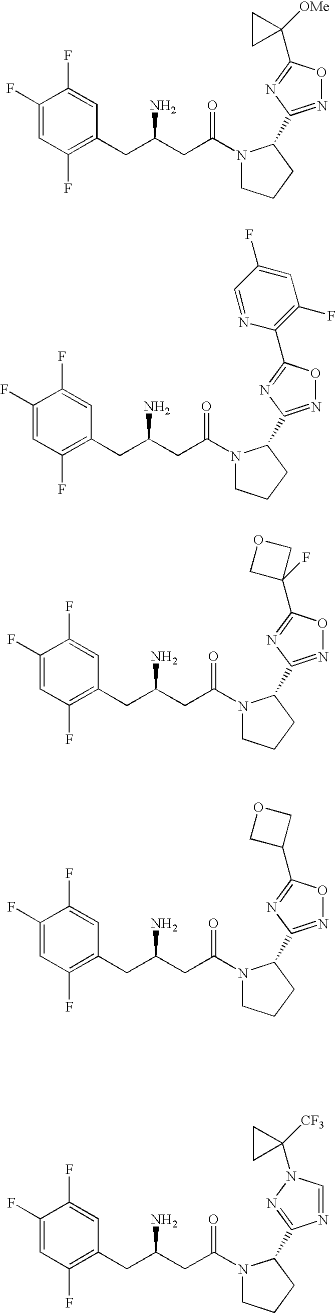

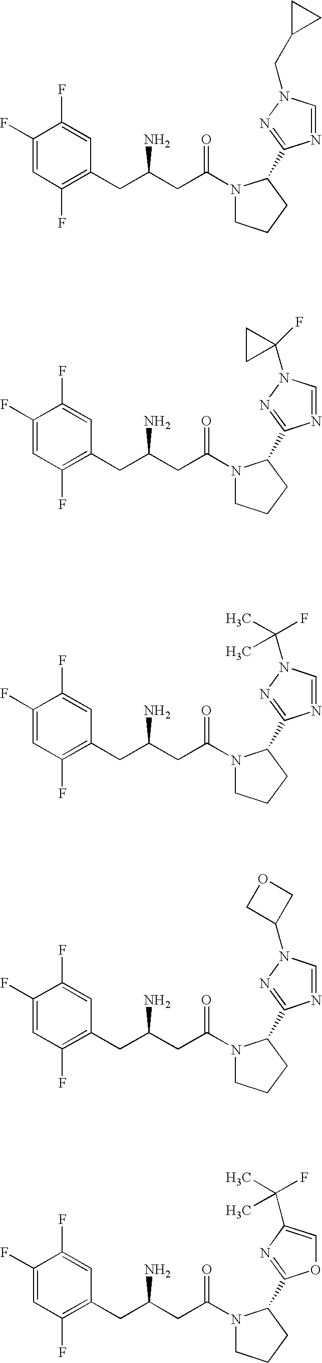

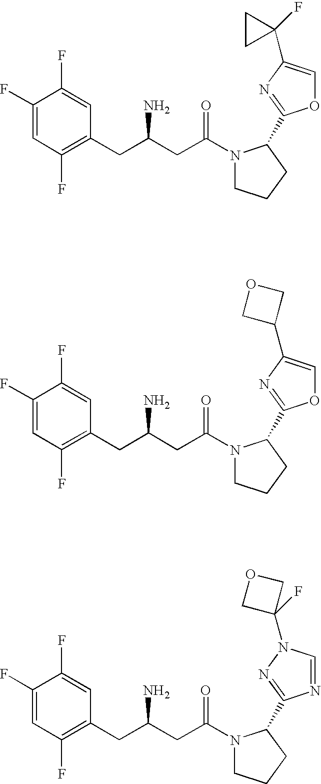

### ASSAYS

Inhibition of DPP-IV peptidase activity was monitored with a continuous fluorimetric assay. This assay is based on the cleavage of the substrate Gly-Pro-AMC (Bachem) by DPP-IV, releasing free AMC. The assay is carried out in 96-well microtiterplates. In a total volume of 100 μl compounds are preincubated with 50 μM DPP-IV employing a buffer containing 10 mM Hepes, 150 mM NaCl, 0.005% Tween 20 (pH 7.4). The reaction is started by the addition of 16 μM substrate and the fluorescence of liberated AMC is detected for 10 minutes at 25° C. with a fluorescence reader (BMG-Fluostar; BMG-Technologies) using an excitation wavelength of 370 nm and an emission wavelength of 450 nm. The final concentration of DMSO is 1%. The inhibitory potential of the compounds were determined. DPP-IV activity assays were carried out with human and porcine DPP-IV (see below); both enzymes showed comparable activities.

Soluble human DPP-IV lacking the transmembrane anchor (Gly31-Pro766) was expressed in a recombinant YEAST-strain as Pre-Pro-alpha-mating fusion. The secreted product (rhuDPP-IV-Gly31-Pro766) was purified from fermentation broth (>90% purity).

In the table are listed the IC₅₀ values for inhibition of DPP-IV peptidase activity determined in assays as described above. The IC₅₀ values were grouped in 3 classes: a≦100 nM; b≧101 nM and ≦1000 nM; c≧1001 nM≦2000 nM.

| Example | IC₅₀ |
|---|---|
| 1 | a |
| 2 | a |
| 3 | a |
| 4 | a |
| 5 | a |
| 6 | a |
| 7 | a |
| 8 | a |
| 9 | a |
| 10 | a |
| 11 | a |
| 12 | a |
| 13 | a |
| 14 | a |
| 15 | a |
| 16 | a |
| 17 | a |
| 18 | a |
| 19 | a |
| 20 | a |
| 21 | a |
| 22 | a |
| 23 | a |
| 24 | a |
| 25 | a |
| 26 | a |
| 27 | a |
| 28 | b |
| 29 | b |
| 30 | b |
| 31 | a |
| 32 | b |
| 33 | a |
| 34 | a |
| 35 | a |
| 36 | a |
| 37 | a |
| 38 | a |
| 39 | a |
| 40 | a |
| 41 | b |
| 42 | b |
| 43 | a |
| 44 | b |
| 45 | a |
| 46 | b |
| 47 | b |
| 48 | a |
| 49 | a |
| 50 | c |
| 51 | c |
| 52 | b |
| 53 | a |
| 54 | a |
| 55 | a |
| 56 | a |

1. A compound of the formula (I) 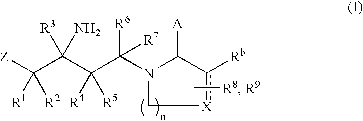
or a pharmaceutically acceptable salt thereof, wherein a dotted line indicates an optionally present double bond and wherein
Z is selected from the group consisting of phenyl; naphthyl; indenyl; C₃₋₇ cycloalkyl; indanyl;
tetralinyl; decalinyl; heterocycle; and heterobicycle, wherein Z is optionally substituted with one or more R¹⁰, wherein R¹⁰ is independently selected from the group consisting of halogen; CN; OH; NH₂; oxo (═O), where the ring is at least partially saturated; R¹¹; and R¹²;
R¹¹ is selected from the group consisting of C₁₋₆ alkyl; O—C₁₋₆ alkyl; and S—C₁₋₆ alkyl, wherein R¹¹ is optionally interrupted by oxygen and wherein R¹¹ is optionally substituted with one or more halogen independently selected from the group consisting of F; and Cl;
R¹² is selected from the group consisting of phenyl; heterocycle; and C₃₋₇ cycloalkyl, wherein R¹² is optionally substituted with one or more R¹³, wherein R¹³ is independently selected from the group consisting of halogen; CN; OH; NH₂; oxo (═O), where the ring is at least partially saturated; C₁₋₆ alkyl; O—C₁₋₆ alkyl; and S—C₁₋₆ alkyl;
R¹, R⁴ are independently selected from the group consisting of H; F; OH; and R¹⁴;
R², R⁵, R⁶, R⁷ are independently selected from the group consisting of H; F; and R¹⁵;
R¹⁴ is independently selected from the group consisting of C₁₋₆ alkyl; OC₁₋₆ alkyl; N(R^{14a})—C₁₋₆alkyl: S—C₁₋₆alkyl: C₃₋₇cycloalkyl; O—C₃₋₇cycloalkyl; N(R^{14a})—C₃₋₇cycloalkyl; S—C₃₋₇ cycloalkyl; C₁₋₆ alkyl-C₃₋₇ cycloalkyl; O—C₁₋₆ alkyl-C₃₋₇ cycloalkyl; N(R^{14a})—C₁₋₆, alkyl-C₃₋₇ cycloalkyl; S—C₁₋₆ alkyl-C₃₋₇ cycloalkyl; heterocycle; O-heterocycle; N(R^{14a})-heterocycle; S-heterocycle: C₁₋₆ alkyl-heterocycle; O—C₁₋₆ alkyl-heterocycle; N(R^{14a})—C₁₋₆ alkyl-heterocycle; S—C₁₋₆ alkyl-heterocycle; wherein R¹⁴ is optionally substituted with one or more halogen independently selected from the group consisting of F; and Cl;
R^{14a} is selected from the group consisting of H; and C₁₋₆ alkyl;
optionally R⁶ is selected from the group consisting of —C(R^{6a}R^{6b})—O—C₁₋₆ alkyl; —C(R^{6a}R^{6b})—O—C₃₋₇ cycloalkyl; —C(R^{6a}R^{6b})—S—C₁₋₆ alkyl; —C(R^{6a}R^{6b})—S—C₃₋₇ cycloalkyl; —C(R^{6a}R^{6b})—N(R^{6c})—C₁₋₆ alkyl; and —C(R^{6a}R^{6b})—N(R^{6c})—C₃₋₇ cycloalkyl, wherein each C₁₋₆ alkyl and C₃₋₇ cycloalkyl is optionally substituted with one or more R^{6d}, wherein R^{6d} is independently selected from the group consisting of halogen; C₁₋₆ alkyl; and C₃₋₇ cycloalkyl;
R^{6a}, R^{6b}, R^{6c} are independently selected from the group consisting of H; and C₁₋₆ alkyl;
R¹⁵ is independently selected from the group consisting of C₁₋₆ alkyl; C₃₋₇ cycloalkyl; and —C₁₋₆ alkyl-C₃₋₇ cycloalkyl, wherein R¹⁵ is optionally substituted with one or more R^{15a}, wherein R^{15a} is independently selected from the group consisting of F; Cl; and OH;
R³ is selected from the group consisting of H; and C₁₋₆ alkyl;
optionally one or more pairs of R¹, R², R³, R⁴, R⁵, R⁶, R^{6a}, R^{6b}, R⁷ independently selected from the group consisting of R¹/R²; R²/R³; R³/R⁴; R⁴R⁵; R⁵/R⁶; R^{6a}/R^{6b} and R⁶/R⁷ form a C₃₋₇ cycloalkyl ring, which is optionally substituted with one or more of R^{15b}, wherein R^{15b} is independently selected from the group consisting of F; Cl; and OH;
Optionally R⁶, R⁷ jointly form an oxo (═O);
n is 0, 1, 2 or 3;
X is selected from the group consisting of —C(R¹⁶R^{c})—; —C(R^{a})═CR^{c}—; C(R¹⁸R^{a})—CR^{c}═, —R¹⁶R^{a})—O—; —C(R¹¹R^{a})—S—; —C(R¹⁶R^{a})—S(O); —C(R¹⁶R^{a})—S(O)₂—; —C(R¹⁶R^{a})—NR^{c}—; and
—C(R¹⁶R^{a})—CR¹⁷R^{c}—;
R⁸ is selected from the group consisting of H; F; OH; and C₁₋₆ alkyl, optionally substituted with one or more halogen selected from the group consisting of F; and Cl;
R⁹, R¹⁶, R¹⁷ are independently selected from the group consisting of H; F; and C₁₋₆ alkyl optionally substituted with one or more halogen selected from the group consisting of F; and Cl;
R^{a}, R^{c} are independently selected from the group consisting of H; F; Cl: and CN;
R^{b}, A are independently selected from the group consisting of H; F; Cl; CN; and A¹, provided that
(a) one of R^{b}, A is A¹; and
(b) when A is A¹ and n is 1 and R⁶, R⁷ jointly form an oxo (═O), X is other than —C(R¹⁶R^{a})—O—; —C(R¹⁶R^{a})—NR^{c}—; or —C(R¹⁶R^{a})—CR¹⁷R^{c}—; and
(c) when A is A¹ and n is 2 and R⁶, R⁷ jointly form an oxo (═O), X is other than —C(R¹⁸R^{c})—;
optionally R^{c} is selected from the group consisting of —O—C₁₋₆ alkyl; —O—C₃₋₇ cycloalkyl; —S—C₁₋₆ alkyl; —S—C₃₋₇ cycloalkyl; —N(R¹⁸)—C₁₋₆ alkyl; and —N(R¹⁸)—C₃₋₇ cycloalkyl, wherein each C₁₋₆ alkyl and C₃₋₇ cycloalkyl is optionally substituted with one or more R^{18a}, wherein R^{18a} is independently selected from the group consisting of halogen; C₁₋₆ alkyl; and C₃₋₇ cycloalkyl, provided that n is 1;
R¹⁸ is independently selected from the group consisting of H; C₁₋₆ alkyl;
Optionally a pair of R^{a}, R^{b}, R^{c} selected from the group consisting of R^{a}/R^{c}; and R^{b}/R^{c} forms a ring Z¹; provided that R^{b}/R^{c} form a ring other than
(a) pyrimidine when R⁶, R⁷ jointly form an oxo (═O) and n is 1 and X is C(R¹⁶R^{a})—C(R^{c})═;
(b) a ring selected from the group consisting of 1,2-diazole; and 1,2,4-triazole when R⁶, R⁷ jointly form an oxo (═O) and n is 1 and X is —C(R¹⁸R^{a})—N(R^{c})—;
Z¹ is selected from the group consisting of Z²; and Z³;
Z² is selected from the group consisting of phenyl; naphthyl; and indenyl; wherein Z² is optionally substituted with one or more R¹⁹; wherein R¹⁹ is independently selected from the group consisting of halogen; CN; COOR²⁰; OR²⁰; C(O)N(R²⁰R^{20a}); S(O)₂N(R²⁰R^{20a}); C₁₋₆ alkyl; O—C₁₋₆ alkyl; S—C₁₋₆ alkyl; COO—C₁₋₆ alkyl; OC(O)—C₁₋₆ alkyl; C(O)N(R²⁰)—C₁₋₆ alkyl; S(O)₂N(R²⁰)—C₁₋₆ alkyl; S(O)N(R²⁰)—C₁₋₆ alkyl; S(O)₂—C₁₋₆ alkyl; S(O)—C₁₋₆ alkyl; N(R²⁰)S(O)₂—C₁₋₆ alkyl; and N(R²⁰)S(O)—C₁₋₆ alkyl; wherein each C₁₋₆ alkyl is optionally substituted with one or more halogen selected from the group consisting of F; and Cl;
Z³ is selected from the group consisting of C₃₋₇ cycloalkyl; indanyl; tetralinyl; decalinyl; heterocycle; and heterobicycle: wherein Z³ is optionally substituted with one or more R²¹, wherein R²¹ is independently selected from the group consisting of halogen; CN; OR²²; oxo (═O), where the ring is at least partially saturated; N(R²²R^{22a}); COOR²²; C(O)N(R²²R^{22a}); S(O)₂N(R²²R^{22a}); S(O)N(R²²R^{22a}); C₁₋₆ alkyl; O—C₁₋₆ alkyl; S—C₁₋₆ alkyl; N(R²²)—C₁₋₆ alkyl; COO—C₁₋₆ alkyl; OC(O)C₁₋₆ alkyl; C(O)N(R²²)—C₁₋₆ alkyl; N(R²²)—C(O)—C₁₋₆ alkyl; S(O)₂N(R²²)—C₁₋₆ alkyl; S(O)N(R²²)—C₆₋₄ alkyl; S(O)₂—C₁₋₆ alkyl; S(O)—C₁₋₆ alkyl; N(R²²)S(O)₂—C₁₋₆ alkyl; and N(R²²)S(O)C₁₋₆ alkyl; wherein each C₁₋₆ alkyl is optionally substituted with one or more halogen selected from the group consisting of F; and Cl;
Optionally R²¹ is C(O)R²², provided that C(O)R²² is bound to a nitrogen, which is a ring atom of a heterocycle or heterobicycle;
R²⁰, R^{20a}, R²², R^{22a} are independently selected from the group consisting of H; C₁₋₆ alkyl; C₃₋₇ cycloalkyl; and —C₁₋₆ alkyl-C₃₋₇-cycloalkyl;
A¹ is selected from the group consisting of phenyl; heterocycle; heterobicycle; C₃₋₇ cycloalkyl, wherein A¹ is substituted with R²³ and wherein phenyl is optionally substituted with one R²⁴ and wherein heterocycle; heterobicycle; C₃₋₇ cycloalkyl are optionally substituted with one R²⁵;
R²⁴ is selected from the group consisting of halogen; CN; COOR²⁶; OC(O)R²⁶; OR²⁶; —C₁₋₆alkyl-OR²⁶; SR²⁶; C(O)N(R²⁶R²⁷); S(O)₂N(R²⁶R²⁷); S(O)N(R²⁶R²⁷); C₁₋₆alkyl; N(R²⁶)S(O)₂R²⁷; and N(R²⁶)S(O)R²⁷; wherein each C₁₋₆ alkyl is optionally substituted with one or more halogen selected from the group consisting of F; and Cl;
R²⁵ is selected from the group consisting of halogen; CN; OR²⁶; —C₁₋₆alkyl-OR²⁶SR²⁶; oxo (═O), where the ring is at least partially saturated; N(R²⁶R²⁷); COOR²⁶; OC(O)R²⁶; C(O)N(R²⁶R²⁷); S(O)₂N(R²⁶R²⁷); S(O)N(R²⁶R²⁷); C₁₋₆ alkyl; N(R²⁶)C(O)R²⁷; S(O)₂R²⁶; S(O)R²⁶; N(R²)S(O)₂R²⁷; and N(R²⁶)S(O)R²⁷; wherein each C₁₋₆ alkyl is optionally substituted with one or more halogen selected from the group consisting of F; and Cl;
Optionally R²⁵ is C(O)R²⁶, provided that C(O)R²⁶ is bound to a nitrogen, which is a ring atom of a heterocycle or heterobicycle;
R²⁶, R²⁷ are independently selected from the group consisting of H; C₁₋₆ alkyl; C₃₋₇ cycloalkyl; and —C₁₋₆ alkyl-C₃₋₇ cycloalkyl; wherein each C₁₋₆ alkyl is optionally substituted with one or more halogen selected from the group consisting of F; and Cl;
R²³ is selected from the group consisting of F; Cl; oxo (═O), where the ring is at least partially saturated; OR^{23a}; N(R^{23a}R^{23b}); C₁₋₆ alkyl, optionally substituted with one or more R²⁸; and —(C(R²⁹R^{29a}))ₘ—W—(C(R³⁰R^{30a}))ₒ-T;
R²⁸ is selected from the group consisting of halogen; CN; COOR³¹; OC(O)R³; OR³¹; SR³¹; C(O)N(R³R³); S(O)₂N(R³¹R³²); S(O)N(R³¹R³²); N(R³¹)S(O)₂R³²; and N(R³¹)S(O)R³²;
R²⁸, R^{29a}, R³⁰, R^{30a} are independently selected from the group consisting of H; F; and R³³;
Optionally one or both pairs of R²⁹, R^{29a}, R³⁰, R^{30a} independently selected from the group consisting of R²⁹/R²⁹; and R³⁰/R^{30a} form a C₃₋₇ cycloalkyl ring, which is optionally substituted with one or more of R³, wherein R³ is independently selected from the group consisting of F; Cl; and OH;
R^{23a}, R^{23b}, R³¹, R³² are independently selected from the group consisting of H; C₁₋₆ alkyl; C₃₋₇ cycloalkyl; and —C₁₋₆ alkyl-C₃₋₇ cycloalkyl; wherein each C₁₋₆ alkyl is optionally substituted with one or more halogen selected from the group consisting of F; and Cl;
R³³ is selected from the group consisting of C₁₋₆ alkyl; C7 cycloalkyl; and —C₁₋₆ alkyl-C₃₋₇ cycloalkyl, wherein R³³ is optionally substituted with one or more R^{33a}, wherein R^{33a} is independently selected from the group consisting of F; Cl; and OH;
m, o are independently selected from the group consisting of 0; 1; 2; and 3;
W is selected from the group consisting of a covalent bond; —O—; —S—; —S(O₂)—; —S(O)—; —N(R³⁴)—; —N(R³⁴)C(O); —C(O)N(R³⁴); —OC(O)—; —C(O)O—; —S(O₂)N(R³⁴)—; —N(R³⁴)S(O)₂—; S(O)N(R³⁴)—; and —N(R³⁴)S(O)—;
R³⁴ is selected from the group consisting of H; C₁₋₆ alkyl; C₃₋₇ cycloalkyl; and —C₁₋₆alkyl-C₃₋₇ cycloalkyl; wherein each C₁₋₆ alkyl is optionally substituted with one or more halogen selected from the group consisting of F; and Cl;
T is selected from the group consisting of H; T¹; and T²;
T¹ is selected from the group consisting of phenyl; naphthyl; and indenyl; wherein T¹ is optionally substituted with one or more R³⁵; wherein R³⁵ is independently selected from the group consisting of halogen; CN; COOR³⁷; OC(O)R³⁷; OR³⁷; —C₁₋₆alkyl-OR³⁷; SR³⁷; S(O)R³⁷; S(O)R³⁷; C(O)N(R³⁷R³⁸); S(O)₂N(R³⁷R³⁸); S(O)N(R³⁷R³⁸); C₁₋₆ alkyl; N(R³⁷)S(O)₂R³⁸; and N(R³⁷)S(O)R³⁸; wherein each C₁₋₆ alkyl is optionally substituted with one or more halogen selected from the group consisting of F; and Cl;
T² is selected from the group consisting of C₃₋₇ cycloalkyl; indanyl; tetralinyl; decalinyl; heterocycle; and heterobicycle; wherein T² is optionally substituted with one or more R³⁶, wherein R³⁶ is independently selected from the group consisting of halogen; CN; OR³⁷; —C₁₋₆alkyl-OR³⁷SR³⁷; oxo (═O), where the ring is at least partially saturated; N(R³⁷R³⁸); COOR³⁷; OC(O)R³⁸; C(O)N(R³⁷R³⁸); S(O)₂N(R³⁷R³⁸); S(O)N(R³⁷R³⁸); C₁₋₆ alkyl; N(R³⁷)C(O)R³⁸; S(O)₂R³⁷; S(O)R³⁸; N(R³⁷)S(O)₂R³⁸; and N(R³⁷)S(O)R³⁸; wherein each C₁₋₆ alkyl is optionally substituted with one or more halogen selected from the group consisting of F; and Cl;
optionally R³⁶C(O)R³⁷, provided that C(O)R³⁷ is bound to a nitrogen, which is a ring atom of a heterocycle or heterobicycle;
R³⁷, R³⁸ are independently selected from the group consisting of H; Cue alkyl; C₃₋₇ cycloalkyl; and —C₁₋₆ alkyl-C₃₋₇ cycloalkyl; wherein each C₁₋₆ alkyl is optionally substituted with one or more halogen selected from the group consisting of F; and Cl.

2. A compound according to claim 1 of formula (Ia) 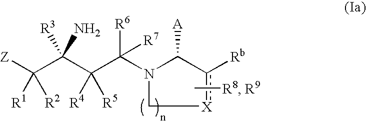
or a pharmaceutically acceptable salt thereof, wherein Z, R¹⁻⁹, n, A, X and R^{b} have the meaning as indicated in claim 1.

3. A compound according to claim 1, wherein Z is selected from the group consisting of phenyl; and heterocycle; and wherein Z is optionally substituted with up to 2 R¹⁰, which are the same or different.

4. A compound according to claim 1, wherein R¹⁰ is selected from the group consisting of F; Cl; CN; and C₁₋₆ alkyl.

5. A compound according to claim 1, wherein R¹, R², R⁴, R⁵ are independently selected from the group consisting of H; F; and C₁₋₆ alkyl, optionally substituted with one or more F.

6. A compound according to claim 1, wherein R³ is H.

7. A compound according to claim 1, wherein R⁶ and R⁷ jointly form an oxo (═O).

8. A compound according to claim 1, wherein R⁶ and R⁷ are independently selected from the group consisting of H; and C₁₋₆ alkyl, optionally substituted with one or more F.

9. A compound according to claim 1, wherein n is 0 and X is —CHR³—CHR^{b}.

10. A compound according to claim 1, wherein n is 1 and X is —CHR^{c}—.

11. A compound according to claim 1, wherein n is 1 and X is —C(R^{a})═CR^{c}— and R^{a}/R^{c} forms a ring Z¹.

12. A compound according to claim 1, wherein n is 1 and X is —CH(R^{a})—CR^{c}═ and R^{a}/R^{c} forms a phenyl.

13. A compound according to claim 1, wherein Z¹ is phenyl.

14. A compound according to claim 1, wherein R⁸, R⁹ are H.

15. A compound according to claim 1, wherein A is A¹.

16. A compound according to claim 1, wherein R^{a}, R^{b}, R^{c} are H.

17. A compound according to claim 1, wherein A¹ is heterocycle.

18. A compound according to claim 1, wherein A¹ is selected from the group consisting of 1,2,4-oxadiazole; 1,2,4-triazole; 1,2,3-triazole; 1,2-diazole; and benzimidazole; wherein A¹ is substituted with R²³ and optionally substituted with R².

19. A compound according to claim 1, wherein R²³ is —(C(R²⁹R^{a}))ₘ—W—(C(R³⁰R^{30a}))ₒ-T.

20. A compound according to claim 1, wherein R²⁵ is Cl.

21. A compound according to claim 1, wherein m and o are 0.

22. A compound according to claim 1, wherein W is a covalent bond.

23. A compound according to claim 1, wherein T is H;

24. A compound according to claim 1, wherein T is phenyl, optionally substituted with one or two R³⁵, which are the same or different.

25. A compound according to claim 1, wherein T is selected from the group consisting of heterocycle; and C₃₋₇ cycloalkyl; wherein T is optionally substituted with one or two R³⁶, which are the same or different.

26. A compound according to claim 25, wherein heterocycle is selected from the group consisting of pyridine; and azetidine and wherein C₃₋₇ cycloalkyl is selected from the group consisting of cyclopropyl; and cyclobutyl.

27. A compound according to claim 1, wherein R³⁵, R³⁶ are independently selected from the group consisting of F; Cl; S(O)₂—C₁₋₆alkyl; —S(O)₂NH₂; —S(O)₂(C₁₋₆ alkyl)₂; —NH—S(O)₂—C₁₋₆ alkyl; and —N(C₁₋₆ alkyl)—S(O)₂—C₁₋₆ alkyl.

28. A compound according to claim 1 selected from the group consisting of

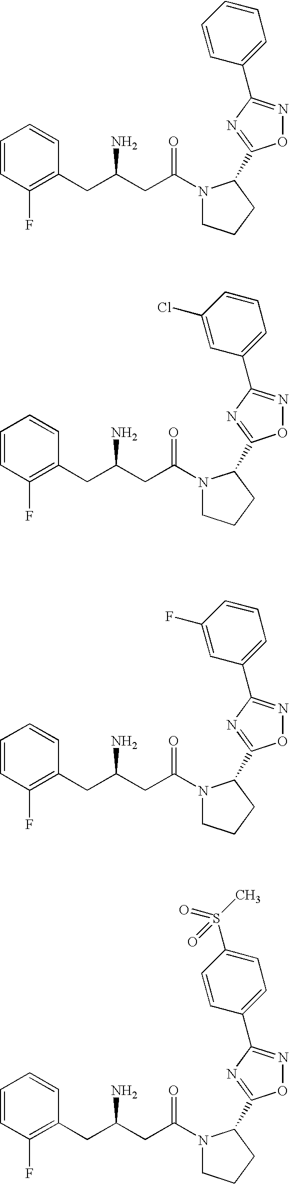

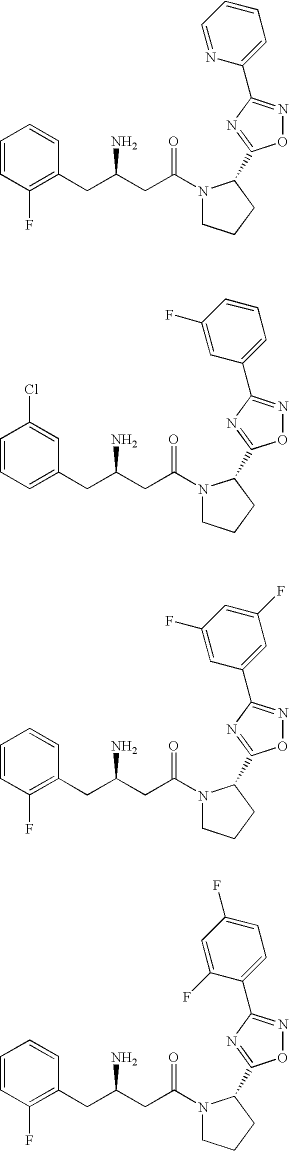

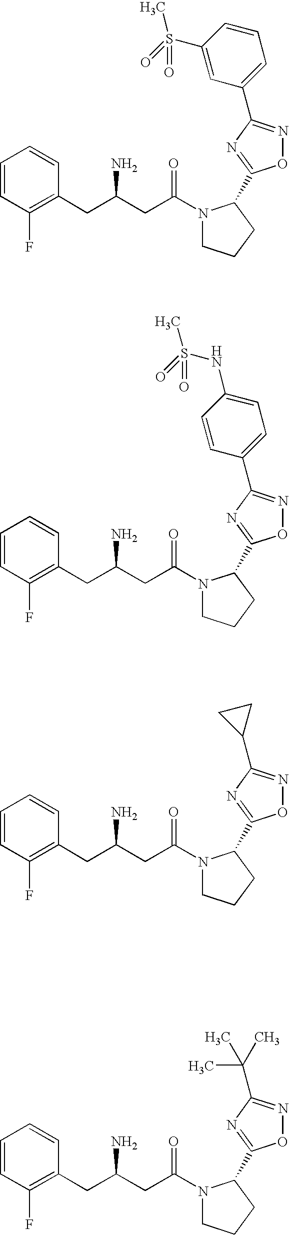

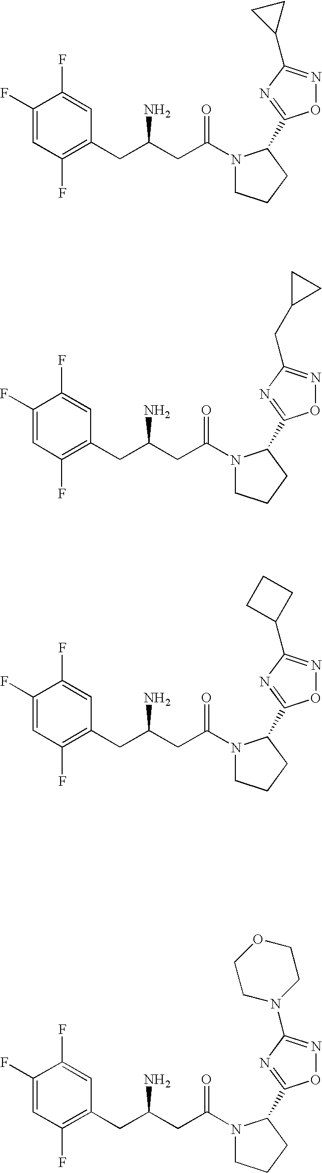

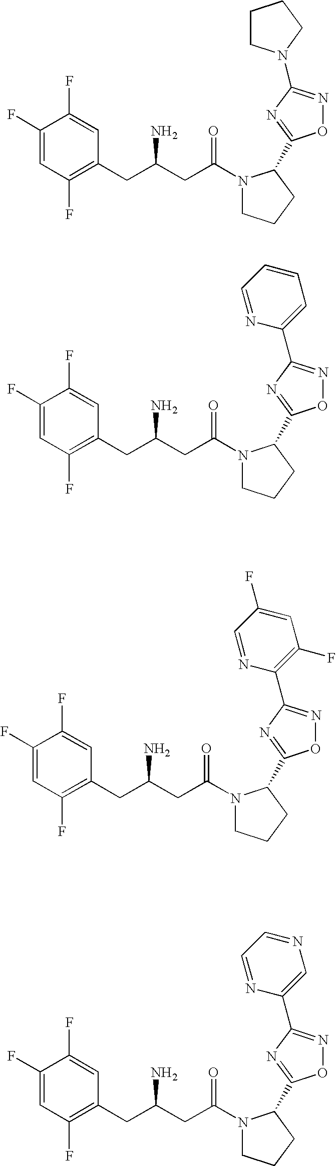

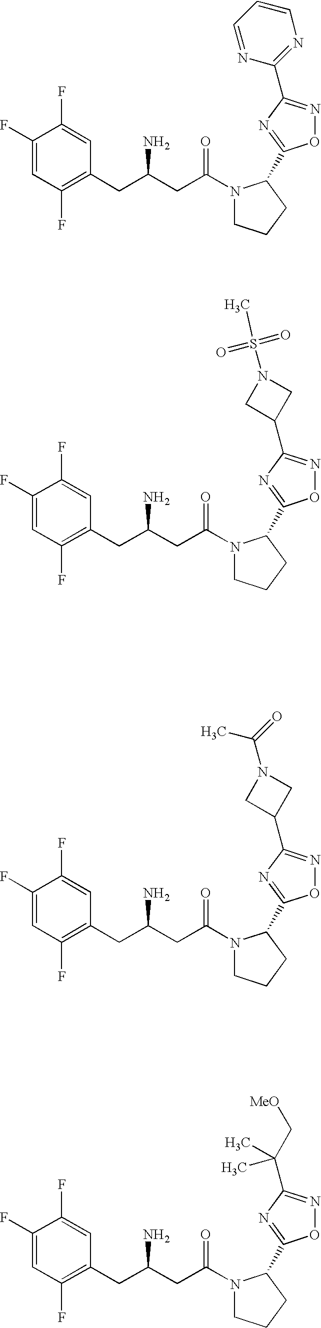

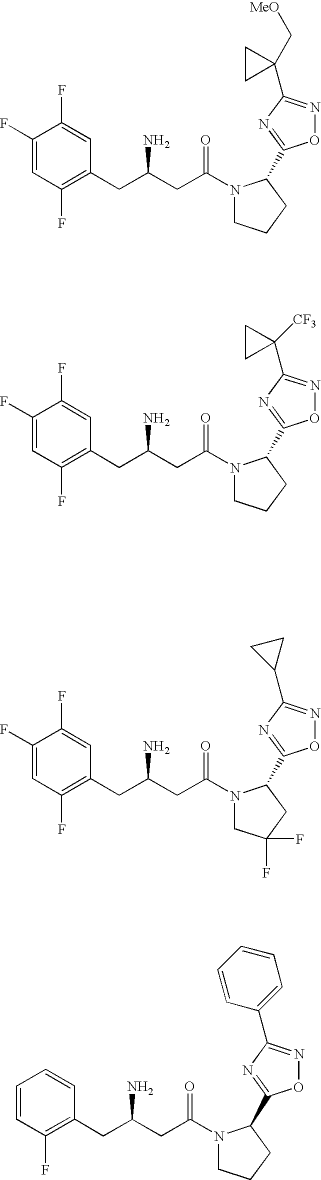

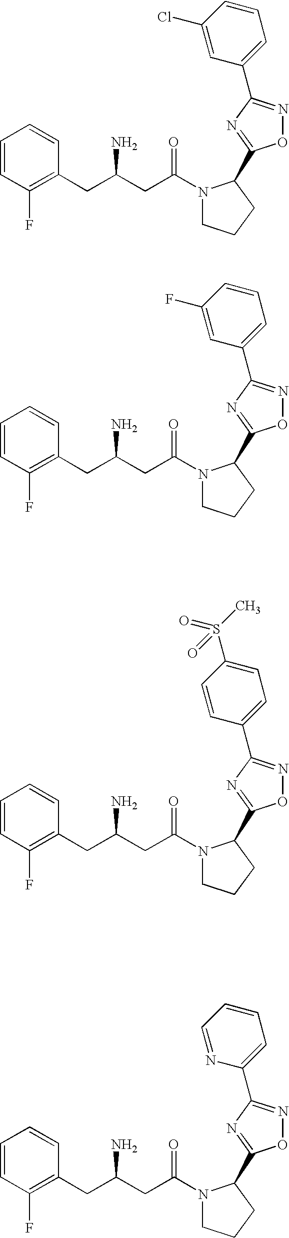

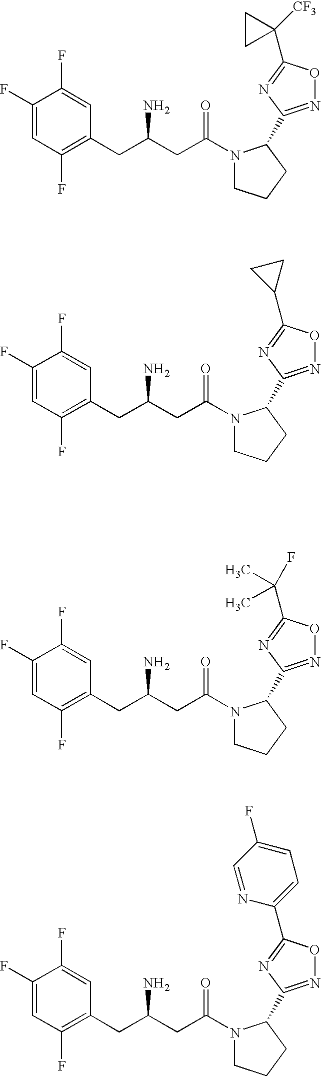

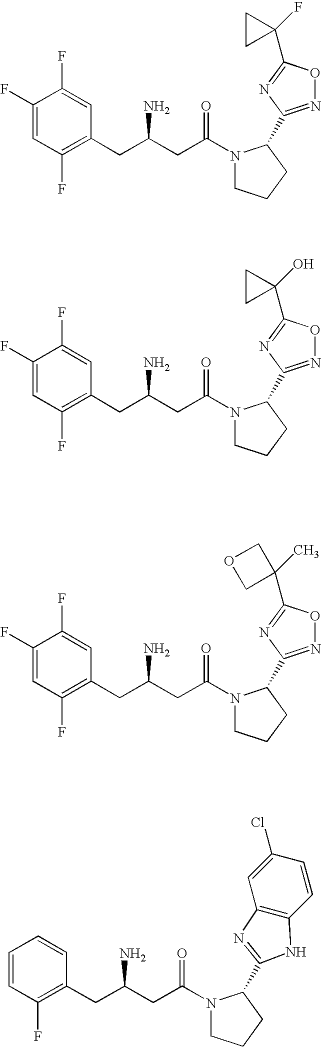

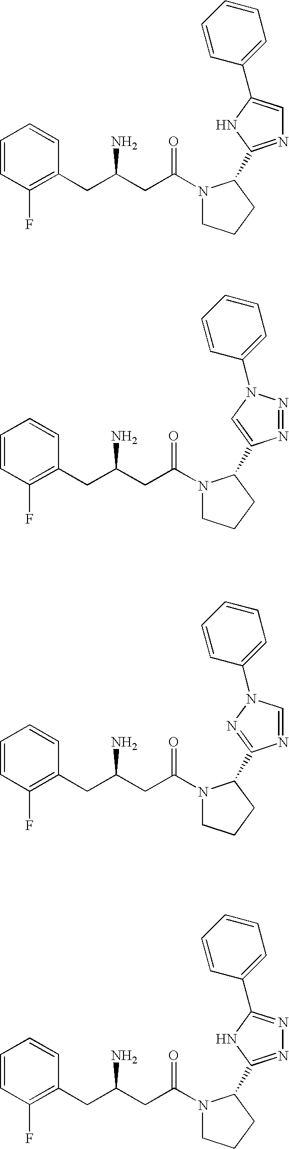

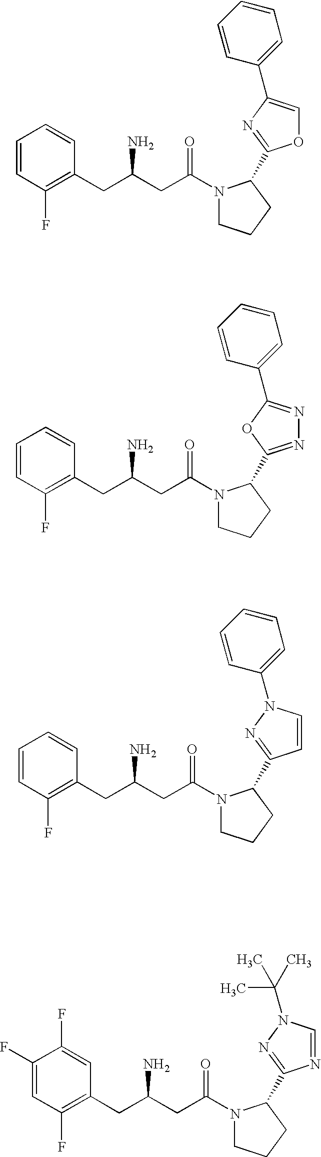

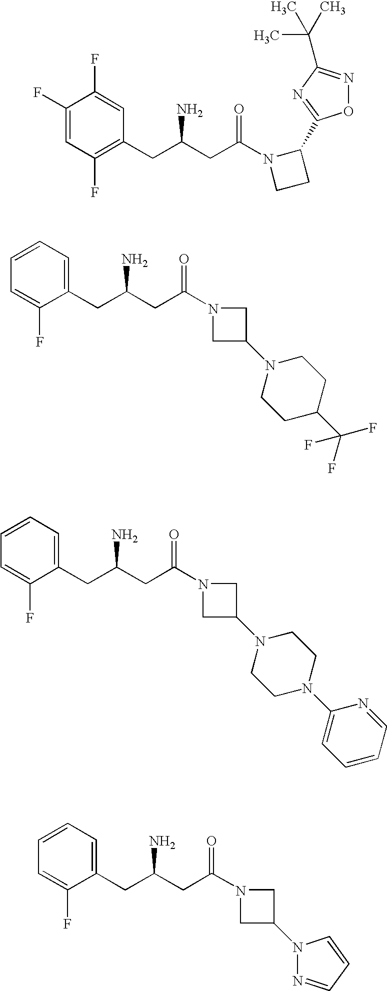

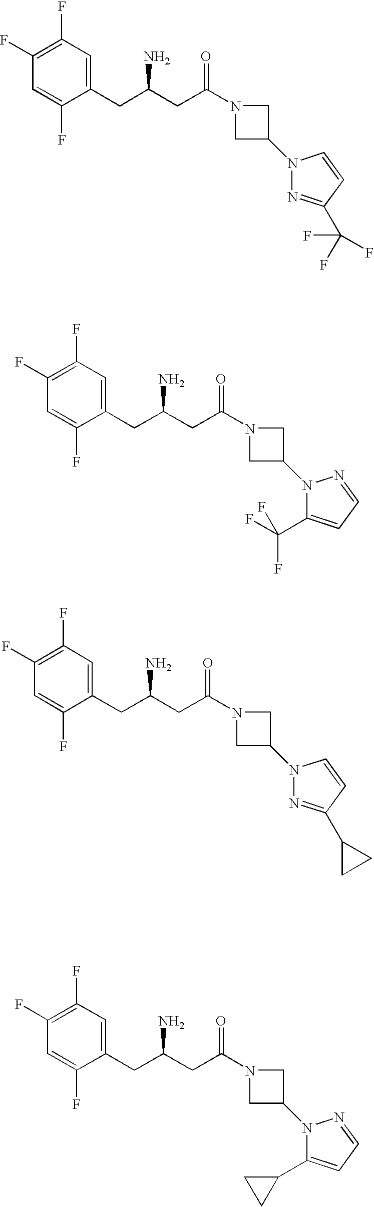

29. A prodrug compound of a compound according to claim 1.

30. A pharmaceutical composition comprising a compound or a pharmaceutically acceptable salt thereof or a prodrug thereof according to claim 1 together with a pharmaceutically acceptable carrier.

31. A pharmaceutical composition according to claim 30, comprising one or more additional compounds or pharmaceutically acceptable salts thereof selected from the group consisting of another of said compound or said pharmaceutically acceptable salt thereof or a prodrug thereof; another DPP-IV inhibitor; insulin sensitizers; PPAR agonists; biguanides; protein tyrosinephosphatase-IB (PTP-1B) inhibitors; insulin and insulin mimetics; sulphonylureas and other insulin secretagogues; α-glucosidase inhibitors; glucagon receptor antagonists; GLP-1, GLP-1 mimetics, and GLP-1 receptor agonists; GIP, GIP mimetics, and GIP receptor agonists; PACAP, PACAP mimetics, and PACAP receptor δ agonists; cholesterol lowering agents; HMG-CoA reductase inhibitors; sequestrants; nicotinyl alcohol; nicotinic acid or a salt thereof; PPARa agonists; PPARoly dual agonists; inhibitors of cholesterol absorption; acyl CoA: cholesterol acyltransferase inhibitors; anti-oxidants; PPAR^{o} agonists; antiobesity compounds; an ileal bile acid transporter inhibitor; and anti-inflammatory agents.

32. A compound or a pharmaceutically acceptable salt thereof or a prodrug thereof of claim 1 for use as a medicament.

33. A method for the treatment or prophylaxis of non-insulin dependent (Type II) diabetes mellitus; hyperglycemia; obesity; insulin resistance; lipid disorders; dyslipidemia; hyperlipidemia; hypertriglyceridemia; hypercholestrerolemia; low HDL; high LDL; atherosclerosis; growth hormone deficiency; diseases related to the immune response; HIV infection; neutropenia; neuronal disorders; tumor metastasis; benign prostatic hypertrophy; gingivitis; hypertension; osteoporosis; diseases related to sperm motility; low glucose tolerance; insulin resistance; ist sequelae; vascular restenosis; irritable bowel syndrome; inflammatory bowel disease; including Crohn's disease and ulcerative colitis; other inflammatory conditions; pancreatitis; abdominal obesity; neurodegenerative disease; retinopathy; nephropathy; neuropathy; Syndrome X; ovarian hyperandrogenism (polycystic ovarian syndrome; Type n diabetes; or growth hormone deficiency, comprising administering to a subject in need of said treatment said compound or said pharmaceutically acceptable salt thereof or a prodrug thereof of claim 1.

34. A method to inhibit DPP-IV peptidase activity comprising administering said compound or said pharmaceutically acceptable salt thereof or a prodrug thereof of claim 1 to a subject in an amount sufficient to inhibit DPP-IV peptidase activity.
